(19) United States
(12) Patent Application Publication
(10) Pub. No.: US 2025/0186730 A1
(43) Pub. Date: Jun. 12, 2025

(54) PATIENT INTERFACE GAS SAMPLING AND ACCESSORY FOR PATIENT INTERFACE

(71) Applicant: Fisher & Paykel Healthcare Limited, Auckland (NZ)

(72) Inventors: Taylor James EDWARDS, Auckland (NZ); Max William Anstiss HAYMAN, Auckland (NZ); Graeme Matthew SMITH, Auckland (NZ); Matthew Jon PAYTON, Auckland (NZ); Samantha Dale OLDFIELD, Auckland (NZ); Craig Karl White, Auckland (NZ); Hamish Adrian OSBORNE, Auckland (NZ); Maxim Darius TOURANI RAD, Auckland (NZ); Samuel Thomas STRUTHERS, Auckland (NZ)

(21) Appl. No.: 18/684,816

(22) PCT Filed: Aug. 24, 2022
(86) PCT No.: PCT/IB2022/057911

§ 371 Date: Feb. 19, 2024

Related U.S. Application Data
(60) Provisional application No. 63/362,486, filed on Apr. 5, 2022.

(30) Foreign Application Priority Data
Aug. 25, 2021 (AU) 2021221742

(51) Int. Cl. A61M 16/08; A61B 5/08; A61B 5/097; A61M 16/06

(52) U.S. Cl. CPC A61M 16/085; A61B 5/097; A61M 16/0666; A61M 16/0683; A61B 5/082

(57) ABSTRACT

The disclosure relates to a patient interface for delivering respiratory support to a patient. The patient interface comprises a gases delivery interface configured to deliver an apparatus gas flow to a patient. The gases delivery interface comprises a delivery outlet to deliver the apparatus gas flow to the patient. The gases delivery interface also comprises a gases delivery side member extending from a first side of the delivery outlet and which comprises an apparatus gases flow path in fluid communication with the delivery outlet. The gases delivery side member comprises a collapsible portion movable upon application of a collapsing force from a normally open configuration to a collapsed configuration in which the apparatus gases flow path is reduced or closed and in order to reduce or stop the apparatus gas flow through the apparatus gases flow path. The patient interface further comprises a gas sampling interface which comprises a sampling inlet configured to receive a patient gas flow at the patient and a sampling outlet configured for delivery of the patient gas flow away from the patient. The gas sampling interface also comprises a sampling conduit in fluid communication with the sampling inlet and the sampling outlet.

### CROSS-REFERENCE

This disclosure claims priority from U.S. provisional patent application 63/362,486 filed on 5 Apr. 2022 and from Australian Patent Application No. 2021221742 filed on 25 Aug. 2021, the contents of each should be understood to be incorporated herein by this reference.

### TECHNICAL FIELD

This disclosure relates to a patient interface for delivering respiratory support to a patient.

### BACKGROUND OF DISCLOSURE

Patients may lose respiratory function during anaesthesia, or sedation, or more generally during certain medical procedures. Prior to a medical procedure a patient may be pre-oxygenated by a medical professional to provide a reservoir of oxygen saturation, and this pre-oxygenation and CO2 flushing/washout may be carried out with high flow respiratory support via a nasal cannula or other patient interface.

In various clinical situations it can be desirable to monitor patient gases such as exhalation gases to monitor O2 content and to monitor whether a patient has become apnoeic. One example situation is where a patient is spontaneously breathing under general anaesthesia or under deep sedation where a patient may drift into and out of apnoea. Another example is where a patient is required to be intubated. In some cases, intubation is completed in 30 to 60 seconds, but in other cases, particularly if the patient's airway is difficult to traverse (for example, due to cancer, severe injury, obesity or spasm of the neck muscles), intubation will take significantly longer. While pre-oxygenation provides a buffer against declines in oxygen saturation, for long intubation procedures, it is necessary to interrupt the intubation process and increase the patient's oxygen saturation to adequate levels. The interruption of the intubation process may happen several times for difficult intubation processes, which is time consuming and puts the patient at serious health risk. After approximately three attempts at intubation the medical procedure, such as an intubation method will be abandoned.

In the event that manual ventilation of the apnoeic, non-intubated, patient is urgently required (such as due to unsuccessful intubation of the patient or a sedated patient that has drifted into apnoea) it is necessary to quickly remove the high flow patient interface and then apply a non-invasive ventilation mask, e.g. a face mask and bag, to the patient. A cannula may be difficult to remove quickly from the patient, for example connectors between headgear and a cannula may be difficult to release quickly or manipulate. Failure to remove the patient interface may result in the seal of the face mask overlying the patient interface or patient interface gases supply tube, disrupting the seal between the face mask and the patient's face. Gases may consequently leak from the face mask during ventilation, rendering ventilation ineffective or inefficient.

In procedures where multiple respiratory support systems are required, there may be a concern that the combination(s) of support systems could cause excessive pressure delivery (for example when a cannula is in place on a patient and an anaesthetist wishes to deliver support through a mask over the top of the cannula). Furthermore, switching between different support systems may be time consuming or difficult.

The above discussion of the background to the disclosure is intended to facilitate an understanding of the disclosure. However, it should be appreciated that the discussion is not an acknowledgement or admission that any aspect of the discussion was part of the common general knowledge as at the priority date of the application.

### SUMMARY

Before turning to a description of the present disclosure, it is useful to provide an explanation of some of the terms that will be used to define the spatial relationship of various parts thereof. Spatial references throughout this specification will generally be based upon a patient interface fitted to a patient's face and configured to deliver respiratory gases from a flow source and through a gases delivery conduit to the patient's nares or mouth. With this environment as the basis, some terms may be defined with reference to the patient such as ‘patient-facing’ and ‘non-patient-facing’. Terms such as ‘inwards’ and ‘outwards’ may be defined with reference to the patient's face. Some terms may be defined with reference to the gases delivery conduit such as ‘behind’ and ‘in front’.

### Patient Interface Gas Sampling

In view of the issues noted in the foregoing, the Applicant has previously developed a patient interface having a collapsible conduit, as described in International Patent application PCT/IB2019/051137 (International Patent Publication WO2019159063). The patient interface provides a means for pre-oxygenating the patient and maintaining the apnoeic window of the patient such as during sedation or under use of general anaesthesia (whether or not intubation is required). The collapsible conduit includes a collapsible portion configured to collapse and temporarily interrupt the (high flow or low flow) respiratory gas flow when, for example, a mask is placed onto a patient's face and a portion of the mask, e.g. the mask cuff, is overlaid across (and pressed onto) the collapsible portion. This allows a clinician to easily apply a non-invasive ventilation mask to the patient over the patient interface when needed and which simultaneously interrupts the high flow patient interface gas flow during application of the ventilation mask.

During use of a patient interface such as the collapsible patent interface described in International Patent application PCT/IB2019/051137 (International Patent Publication WO2019159063), it is beneficial to monitor gases at the patient where the patient is receiving respiratory support. Gas monitoring provides useful feedback to clinicians and, for example, during the pre-oxygenation phase to determine whether the patient has reached a desired end expiratory O2 level to indicate that pre-oxygenation is complete. Patent gas monitoring may also include monitoring of CO2 and/or volatiles. Such gas monitoring may also be useful to determine the patient condition or a change in the patient condition when the patient has diminished respiratory function or a risk of diminished respiratory function, for example during a medical procedure when anaesthetic agents are used as described above. Such change in patient condition can include a spontaneously breathing patient becoming apnoeic or experiencing a blocked (non-patent) airway, such that an interruption of the high flow respiratory support is required as discussed above.

The Applicant has previously developed a gas sampling device, as described in International Patent application PCT/NZ2017/050134 (International Patent Publication WO2018070885). This device includes a moveable inlet which can be positioned near a patient's nose or mouth to sample patient gases. A conduit may connect the sampling device to a respiratory gases monitor (for example, a capnography device) which provides a monitor of the sampled gases. The sampling device has been designed to be attachable to a standard nasal cannula but is not configured to operate with a collapsible patient interface of the type disclosed in International patent application PCT/IB2019/051137 (International Patent Publication WO2019159063).

Accordingly, it is desirable to provide a new or alternative patient interface with a collapsible portion and which can also facilitate patient gas sampling.

According to an aspect of the disclosure, there is provided a patient interface comprising:
a gases delivery interface configured to deliver an apparatus gas flow to a patient, the gases delivery interface comprising:
   a delivery outlet to deliver the apparatus gas flow to the patient; and
   a gases delivery side member extending from a first side of the delivery outlet and comprising an apparatus gases flow path in fluid communication with the delivery outlet, the gases delivery side member comprising a collapsible portion movable upon application of a collapsing force from a normally open configuration to a collapsed configuration in which the apparatus gases flow path is reduced or closed and in order to reduce or stop the apparatus gas flow through the apparatus gases flow path, and the patient interface further comprising:
a gas sampling interface comprising:
   a sampling inlet configured to receive a patient gas flow at the patient;
   a sampling outlet configured for delivery of the patient gas flow away from the patient; and
   a sampling conduit in fluid communication with the sampling inlet and the sampling outlet, the sampling conduit configured to remain open to maintain fluid communication between the sampling inlet and sampling outlet when the collapsible portion is moved to the collapsed configuration.

The patient interface according to the first aspect of the disclosure may advantageously facilitate sampling of patient gases whilst the collapsible gases delivery interface is concurrently operated. In particular, the sampling conduit is able to continue sampling whilst the apparatus gases of the gases delivery interface are reduced or stopped during movement of the collapsible portion to the collapsed configuration. The sampling conduit may be configured for delivering patient gases to a gas sensor or gas monitoring device. The sampling conduit may be configured for delivering patient gases to a sensor or monitoring device located within the patient gas flow. The sensor may be located within the sampling conduit. The sampling conduit could be configured to deliver patient gases to a respiratory gas monitor in fluid communication with the sampling conduit via the sampling outlet.

A patient interface according to the first aspect of the disclosure thereby facilitates continuous monitoring of the patient gases to provide feedback for clinicians both during respiratory support (for example high-flow respiratory support) provided via the gases delivery interface and also during interruption of the apparatus gas flow through the gases delivery interface (for example, during application of a patient face mask onto the patient's face).

It will be appreciated that references herein to “apparatus gas flow” refers to a gas flow from an apparatus such as a respiratory support system which could comprise a flow generator or a wall source, a compressed air source or any other suitable respiratory gas source. Apparatus gas flow may therefore include a respiratory gas flow. Apparatus gas flow could also include anaesthetic agents or oxygen delivered to the patient.

The sampling inlet is configured to receive a patient gas flow at the patient which may include expiratory gases from the patient and/or inhalation gases for the patient and/or apparatus gases from the apparatus gas flow and/or atmospheric gases or a combination of two or more thereof. The atmospheric gases which may be sampled via the sampling inlet could include entrained atmospheric gases contained within the patient's expiratory gases and/or atmospheric gases present in front of the patient's face or within the patient's airway that have not been exhaled from the patient. The patient gases sampled may include apparatus gases which have been delivered to the patient and which are yet to be inhaled. The patient gases could also include other gases such as anaesthetic agents being delivered to the patient.

It will be appreciated that atmospheric gases, expiratory gases, apparatus gases and anaesthetic gases may become mixed at the sampling inlet and therefore the patient gases received by the sampling inlet may typically comprise a mixture of two or more of these or other gases. Similarly, it will be appreciated that the patient gas flow received by the sampling inlet could in some instances comprise only one of these types of gases. The patient gas flow can vary with time (for example, between patient exhalations) and therefore the composition of the patient gas flow received by the sampling inlet can vary with time.

The patient gas flow received at the patient may be received from a patient's airway. The patient gas flow received at the patient may be received in front of a patient's mouth and/or nose. The patient gas flow received at the patient may be received internally of the patient. For example, the patient gas flow may be received from inside the patient's mouth and/or nose.

The sampling outlet may be configured for fluid communication with a respiratory gas monitor. For example, the sampling outlet may be connected via a tube or other conduit to a gas monitoring device capable of analysing the patient gases received through the sampling inlet. The gas sampling interface may therefore be configured for sidestream capnography. In this instance, the sampling outlet may therefore facilitate delivery of the patient gas flow away from the patient and towards the respiratory gas monitor.

In an embodiment, the gas sampling interface may include a gas sensor in or at the sampling conduit and/or in or at the sampling outlet. For example, the gas sampling interface may be configured for mainstream capnography. The gas sensor may be connected via a wired or wireless data communication to an appropriate receiver capable of displaying the sensor data to a clinician. In this instance, the sampling outlet may vent patient gas flow to ambient once downstream of the sensor and in this manner the sampling outlet is configured to deliver the patient gas flow away from the patient in order to enable flow through the conduit and past the sensor.

In another alternative embodiment, the gas sampling interface could include a passive sampling configuration and, for example, could be configured to sample the patient gases via colourimetry. In this instance, the sampling conduit could be configured to deliver patient gases to an assay of colorimetric reagents or to another form of colourimeter configured to indicate the presence or concentration of one or more particular gases in the patient gas flow. In some configurations, the gas sampling interface may include the colourimetry means in the sampling conduit or at the sampling outlet.

The collapsible portion of the gases delivery side member may be moved from a normally open configuration to a collapsed configuration. An open configuration will be understood as meaning a configuration in which the collapsible portion is capable of delivering apparatus gases to the patient at the desired flow rate. For example, the open configuration may involve a lumen of the gases delivery member being sufficiently open and/or unobstructed to allow for the required apparatus gas flow rate to be delivered to the patient. The collapsible portion is configured to be in a ‘normally’ open configuration which will be understood as meaning the collapsible portion will default to the open configuration or is in the open configuration ‘at rest’. For example, the collapsible portion can remain in (and will return to) the open configuration in the absence of an external force urging the collapsible portion away from the open configuration. In other embodiments, the collapsible portion may be self-collapsing or be in a ‘normally’ closed configuration. That is, it may be partially or fully collapsed (collapsed configuration) when there is no apparatus gas or a low flow of apparatus gas flowing through it, and expands (open configuration) when there is some level of apparatus gas flowing through it. However, a ‘normally’ open collapsible portion (in an open configuration in the absence of an external force urging the collapsible portion away from the open configuration) can be beneficial in that a substantial amount of flow and/or pressure is not needed to first open or maintain the collapsible portion in an open configuration for delivery of the apparatus gas to the patient.

The normally open configuration of the collapsible portion may be achieved via inherent material properties of the collapsible portion or via geometry or other structural configuration of the collapsible portion. For example, the collapsible portion may be formed of a flexibly resilient material which can be folded, flattened or otherwise temporarily manipulated by an external force but urges toward its original state, position or configuration upon cessation or release of the external force.

The ‘collapsed configuration’ of the collapsible portion will be understood as meaning a configuration in which the collapsible portion is physically manipulated or affected so as to reduce the path of apparatus gases through the collapsible portion. Upon movement to the collapsed configuration, the collapsible portion may undergo a change in cross-section. For example, a reduction in cross-section to as to produce a more restricted or convoluted flow path. The collapsed configuration may involve an internal passageway such a lumen becoming reduced in cross-section so as to occlude, obstruct or otherwise reduce flow.

Movement of the collapsible portion to the collapsed configuration may involve the collapsible portion being folded, bent, kinked, flattened, compressed or twisted into the collapsed configuration. The collapsed configuration may involve one or more side portions of the collapsible portion moving toward or against each other so as to occlude or obstruct the apparatus gas flow path. The collapsed configuration could reduce the flow of apparatus gases to substantially zero or, alternatively, could reduce the flow of apparatus gases as compared to the open configuration but with some residual flow still occurring in the collapsed configuration.

In an embodiment of the disclosure, the collapsible portion is resiliently deformable from the normally open configuration to the collapsed configuration. For example, two or more side portions of the collapsible portion may be configured for resilient deformation towards or against each other under influence of the collapsing force and to return to a normal position spaced from one another upon removal of the collapsing force. The movement towards or against each other may be relative movement towards one another. That is, one of the side portions may remain static relative to the patient interface (and to the patient) and the other of the side portions may move toward or against the static side portion. In an alternative embodiment of the disclosure, the collapsible portion comprises a side portion configured for resilient deformation towards or against another side portion of the collapsible portion.

The side portions may be located adjacent to or may be connected to one another. For example, side portions may be connected at a fold line and may be folded toward or against one another. Alternatively, the side portions may be opposite to one another. For example, opposite sides of the collapsible portion could be moved toward one another.

The resilient deformation of the collapsible portion may comprise bending or folding or one or more sides of the collapsible portion. The collapsible portion may comprise a wall of non-uniform thickness. For example, the collapsible portion may include a thin-walled portion which facilitates folding or bending of the collapsible portion at the thin-walled portion. The collapsible portion may include a single thin-walled portion. The thin-walled portion may comprise a relative thin section of the wall which forms a hinge portion or collapsing portion of the wall which induces folding to occur at the thin-walled portion upon application of the collapsing force to the collapsible portion. The relatively thin section of the thin-walled portion allows the section of wall to be particular adapted to fold or bend at the fold points so as to transition between the open and closed configurations. In this way, the collapsible portion preferentially bends or folds at the fold points to move between the open and closed configurations.

According to an embodiment of the disclosure, the collapsible portion comprises a pair of thin-walled portions configured to provide fold lines at which the collapsible portion folds or bends upon application of the collapsing force. The pair of thin-walled portions may be located on opposite sides of the collapsible portion or could be otherwise relatively located, for example adjacent to one another.

The collapsible portion comprises a portion of the gases delivery side member which may comprise a conduit. The collapsible portion may therefore itself comprise a conduit, tube or other structure configured to deliver gases. The collapsible portion could have a circular cross-section. The collapsible portion could have an elongate cross section such as an oval or ovoid cross-section or a stadium cross-section. According to an embodiment of the disclosure, the collapsible portion has an elongate cross-section which comprises a pair of longitudinal sides extending between a pair of ends and wherein the thin-walled portions are located at the ends. In a particular embodiment, each of the ends comprises a single thin-walled portion. Each of the ends may include more than one thin-walled portion.

The provision of a thin-walled portion at the ends of the elongate cross-section may advantageously promote folding or bending at the ends resulting in one or both of the longitudinal sides moving toward the other longitudinal side. The collapsible force may be applied to a first of one of the longitudinal sides and in the direction of the other longitudinal side which results in a flattening of the collapsible portion as the first of longitudinal sides is moved toward or against the second longitudinal side.

Various embodiments of possible collapsible portion configurations are discussed in Applicant's previous international patent publications WO2018/029638 and WO2019159063.

According to the first aspect of the disclosure, the gas sampling interface could be provided to the gases delivery side member or could be separate to the gases delivery side member. In an embodiment of the disclosure, the sampling conduit extends from a second side of the delivery outlet that is opposite to the first side. For example, the gases delivery conduit may be separate from the gases delivery side member and may extend from the opposite side of the delivery outlet and/or toward an opposite side of the patient's face as compared to the gases delivery side member.

The sampling conduit may have a dual function in that it may also be used as a structural component to support part of the patient interface. For example, the sampling conduit may be used with or as part of a head strap which connects with the patient's head to secure the patient interface in position relative to the patient's face. In an embodiment, the sampling conduit has an end configured for coupling to a headstrap and comprises an internal passageway providing the fluid communication between the sampling inlet and sampling outlet, the sampling conduit also comprising a patient-facing wall and a non-patient-facing wall.

Alternatively, the sampling conduit may not contribute to securing of the patient interface and the delivery outlet may be supported by a component other than the sampling conduit. For example, in an embodiment, the patient interface further comprises a non-delivery side member extending from a second side of the delivery outlet that is opposite to the first side and the non-delivery side member having a headstrap end that is configured for coupling to a headstrap, the non-delivery side member also comprising a patient-facing wall and a non-patient-facing wall.

The “non-delivery” side member will be understood as meaning a side member which is not configured for delivering the apparatus gases to the patient. That is, the non-delivery side member does not form part of the apparatus gases flow path which is provided by the gases delivery side member.

Whilst the non-delivery side member is unassociated with the apparatus gas flow, the non-delivery side member may be associated with the patient gas flow. For example, the sampling conduit may be associated with the non-delivery side member. In an embodiment, the sampling conduit is provided on the non-delivery side member.

The provision of the sampling conduit to the non-delivery side member can be provided by various configurations.

In an embodiment, the sampling conduit extends through a portion of the non-delivery side member.

In an embodiment, the sampling conduit extends through an internal passage of the non-delivery side member.

In an embodiment, the sampling conduit extending between a pair of spaced apart openings in one or more walls of the non-delivery side member.

In an embodiment, the spaced apart openings comprising an inlet port proximate to the delivery outlet and an outlet port proximate to the headstrap end.

In an embodiment, the inlet port and/or the outlet port is located in the patient-facing wall of the non-delivery side member.

In an embodiment, the inlet port and/or the outlet port is located in the non-patient-facing wall of the non-delivery side member.

In an embodiment, the sampling inlet is located at or adjacent the inlet port and the sampling outlet located at or adjacent the outlet port.

In an embodiment, the sampling conduit comprises a tube extending through both the inlet port and the outlet port and through the internal passage which extends between the inlet and outlet ports.

In an embodiment, the sampling conduit comprises the internal passage of the non-delivery member and wherein the inlet and/or outlet ports are configured for connection to respective sampling tubes configured to extend from the inlet and outlet ports to the sampling inlet and sampling outlet respectively.

In an embodiment, the inlet and/or outlet ports are configured for connection to sampling tubes via a luer lock or threaded connection or plug-fit or barb.

In an embodiment, the non-delivery member comprises a sampling inlet tube moulded to the sampling inlet and/or a sampling outlet tube moulded to the sampling outlet.

In an embodiment, the sampling conduit is integrally formed with the non-delivery side member.

In an embodiment, the sampling conduit being attachable with the non-delivery side member. In an embodiment, the non-delivery side member comprising a channel configured to receive the sampling conduit and permitting removable attachment between the sampling conduit and the non-delivery side member.

In an embodiment, the channel being located in the patient-facing wall of the non-delivery side member. In an embodiment, the channel being is located in the non-patient-facing wall of the non-delivery side member.

In an embodiment, the sampling inlet is proximate to the delivery outlet and the sampling outlet is proximate to the headstrap end of the non-delivery side member.

In an embodiment, the sampling inlet and sampling outlet are separated by a distance that is greater than a width of a section of a face mask seal that is configured for placement on the patient's face and to bear over a portion of the non-delivery side member.

In an embodiment, the non-delivery side member is configured to resist deformation upon bearing of the face mask seal over the non-delivery member.

In an embodiment, the non-delivery side member is configured to deform upon bearing of the face mask seal over the non-delivery side member and wherein the sampling conduit remains open during deformation of the non-delivery side member.

In an embodiment, the patient-facing wall has a more pronounced curve as compared to the non-patient-facing wall.

In an embodiment, the non-delivery side member has an elongate cross-section which is asymmetric in at least one axis.

In an embodiment, the cross-section is asymmetric in an axis substantially parallel with the patient-facing wall.

In an embodiment, the patient-facing wall has a different curvature configuration to the non-patient-facing wall.

In an embodiment, the non-delivery side member has an asymmetric lens cross section.

In an embodiment, the non-delivery side member cross-section comprises a pair of opposite and spaced apart edges and wherein the patient-facing wall and the non-patient-facing wall extend between the pair of edges.

In an embodiment, the patient-facing wall has a substantially convex formation extending between the pair of opposite edges.

In an embodiment, the non-patient-facing wall has a substantially planar formation extending between the pair of opposite edges.

For example, in an embodiment, the sampling conduit extends through a portion of the non-delivery side member. The sampling conduit may extend through an internal passage of the non-delivery side member. As noted above, the non-delivery side member may be unassociated with the apparatus gas flow and therefore the internal passage of the non-delivery side member may be unconfigured for delivering apparatus gases.

The sampling conduit may have any suitable diameter or profile. For example, any suitable diameter or profile internal or external to the non-delivery side member. The sampling conduit may have a uniform cross-section. The sampling conduit may have a varying cross-section. The varying cross-section could increase or decrease along the length of the sampling conduit.

In an embodiment, the sampling conduit may extend between a pair of spaced apart openings in one or more walls of the non-delivery side member. The spaced apart openings may comprise an inlet port proximate to the delivery outlet and an outlet port proximate to the headstrap end of the non-delivery side member. The inlet and outlet ports could be provided in a common wall, side or side portion of the non-delivery side member or could alternatively be on different walls, sides or side portions of the non-delivery side member. The inlet and/or the outlet port may be located in the patient-facing wall of the non-delivery side member. The inlet and/or the outlet port may be located in the non-patient-facing wall of the non-delivery side member.

The sampling inlet may be located sufficiently close to the patient's airway so as to receive the patient airflow which can include expiratory gases. The sampling outlet may be located to facilitate connection with a respiratory gas monitor.

In an embodiment, the sampling inlet is located at or adjacent the inlet port and the sampling outlet is located at or adjacent the outlet port. In an embodiment, the sampling inlet consists of the inlet port and/or the sampling outlet consists of the outlet port. In an alternative embodiment, the sampling conduit comprises a tube extending through both the inlet port and the outlet port and through the internal passage which extends between the inlet and outlet ports.

In an embodiment, the sampling conduit comprises the internal passage of the non-delivery member and wherein the inlet and/or outlet ports are configured for connection to respective sampling tubes configured to extend from the inlet and outlet ports to the sampling inlet and sampling outlet respectively.

In an embodiment, the sampling inlet could be located at (or consist of) the inlet port in which case there may only an outlet tube connecting the sampling outlet to the outlet port.

In an alternative embodiment, the sampling outlet could be located at (or consist of) the outlet port in which case there may only an inlet tube connecting the sampling outlet to the outlet port.

In an embodiment, the inlet and/or outlet ports are configured for connection to sampling tubes via a luer lock or threaded connection or plug-fit or a barb. The inlet port could utilise a different type of connection from the outlet port. This may advantageously improve usability by preventing components or tubes from being incorrectly connected to the wrong port.

In an embodiment, the non-delivery member comprises a sampling inlet tube moulded to the sampling inlet and/or a sampling outlet tube moulded to the sampling outlet. The sampling inlet tube may be integrally formed with the sampling inlet and or the inlet port. The sampling outlet tube may be integrally formed with the sampling outlet and/or the sampling outlet.

In an embodiment, the sampling conduit is integrally formed with the non-delivery side member. In an alternative embodiment, the sampling conduit is attachable with the non-delivery side member. The sampling conduit may be removably attached or connected with the non-delivery side member. The non-delivery side member may be configured for removable connection with the sampling conduit. For example, the non-delivery side member may comprise a channel configured to receive the sampling conduit and permitting removable attachment between the sampling conduit and the non-delivery side member. The channel may be located in an external surface of the non-delivery side member. The channel may have a formation which corresponds to the formation of the sampling conduit. For example, the channel may have a width or diameter approximately commensurate with a diameter of the sampling conduit so as to snugly receive and retain the sampling conduit in the channel. The channel may have a semi-circular cross-section. The sampling conduit may be frictionally retained with the channel. The channel may be configured in geometry to retain the sampling conduit in the channel. For example, the channel may have a width or diameter slightly less than a diameter of the sampling conduit such that the channel is resiliently expanded when the sampling conduit is fitted in the channel and retained therein by resilient ‘squeezing’ of the channel onto the sampling conduit. The sampling conduit may be formed of a more rigid material than the portion of the non-delivery member in which the channel is formed so as to avoid deformation of the sampling conduit when fitted into a channel of slightly smaller size than the sampling conduit.

In an embodiment, the channel is located in the patient-facing wall of the non-delivery side member. In an alternative embodiment, the channel is located the non-patient-facing wall of the non-delivery side member. The channel advantageously allows for convenient attachment and detachment of the sampling conduit and whereby the sampling conduit can be fitted to the non-delivery side member when required and removed when not required and/or to facilitate maintenance, cleaning or replacement of certain components.

In an embodiment, the sampling inlet is proximate to the delivery outlet and the sampling outlet is proximate to the headstrap end of the non-delivery side member. The spacing between the sampling inlet and sampling outlet may of course vary. In an embodiment, the sampling inlet and sampling outlet are separate by a distance that is greater than a width of a section of a face mask seal that is configured for placement on the patient's face and to bear over a portion of the non-delivery side member. This may advantageously allow for a patient face mask to be positioned over the delivery outlet and the sampling inlet and for the sampling outlet to be located outside of the patient face mask. The positioning of the face mask onto the non-delivery side member may therefore not interfere with the sampling outlet and its fluid connection with the respiratory gas monitor.

As noted, the sampling conduit of the first aspect of the disclosure is configured to remain open when the collapsible portion is moved to the collapsed configuration. The bearing of the face mask seal over the non-delivery member applies a force to the non-delivery side member which may be transmitted or directly applied to the sampling conduit. Accordingly, the sampling conduit may be configured to remain open under application or influence of the face mask force. The sampling conduit may therefore allow for continuous patient gas sampling.

In an embodiment, the non-delivery side member may itself be configured to resist deformation upon bearing of the face mask seal over the non-delivery side member. Alternatively, the non-delivery side member may be configured to deform upon bearing of the face mask seal over the non-delivery side member and wherein the sampling conduit remains open during deformation of the non-delivery side member. The deformation of the non-delivery side member may involve a flattening of the non-delivery side member. This may advantageously help to form a seal between the face mask and the patient's face and/or between the face mask and the non-delivery side member. For example, the non-delivery side member may be configured to move to a flattened configuration and the face mask can resiliently deform around the flattened configuration to form a seal with the patient's face. The patient interface may be configured to reduce or avoid leaks when a face mask is placed over the patient interface, which could lead to undesirable dilatation of the respiratory support and/or the patient gas flow which is being sampled.

The non-delivery side member may be hollow (e.g. tubular) or may be non-hollow. The non-delivery side member could comprise a conduit or tube. The non-delivery side member could have a circular cross-section or could have a non-circular cross section. The non-delivery side member could have an elongate cross section such as an oval or ovoid cross-section or a stadium cross-section. According to an embodiment of the disclosure, the non-delivery side member has an elongate cross-section which comprises a pair of longitudinal sides extending between a pair of ends. The ends may be rounded for example semi-circular. Alternatively, the ends may comprise edges (for example, angular edges) and at which the longitudinal sides meet one another.

The non-delivery side member may have a symmetrical cross-section. For example, a circular, oval or stadium cross-section which is symmetrical about a length axis and/or a width axis.

Alternatively, in an embodiment the non-delivery side member has an elongate cross-section which is asymmetric in at least one axis. In an embodiment, the elongate cross-section may comprise a length axis and a width axis and the cross-section is asymmetric about the length axis. The cross-section may include a pair of longitudinal sides extending between a pair of end edges and wherein the longitudinal sides are asymmetric. For example, one of the side portions may include a channel configured to receive the sampling conduit. One of the sides may have a different curve configuration from the other side. In an embodiment, the patient-facing wall has a different curvature configuration to the non-patient-facing wall. In an embodiment, the patient-facing wall of the non-delivery side member has a more pronounced curve as compared to the non-patient-facing wall. In an embodiment, the cross-section is asymmetric in an axis substantially parallel with the patient-facing wall.

The non-delivery side member cross-section may have an asymmetric lens shaped or an air foil shape. For example, the cross-section may comprise two longitudinal sides having different levels of curvature and which meet at opposite end edges. Each of the longitudinal sides may have a convex configuration (i.e. bulging outward). The two longitudinal curved sides may comprise the patient-facing side and the non-patient-facing side. In an embodiment, the non-patient-facing side has a low level of curvature and may be substantially flat and the patient-facing side has a higher level of curvature.

In an embodiment, the non-delivery side member cross-section comprises a pair of opposite and spaced apart edges and wherein the patient-facing wall and the non-patient-facing wall extend between the pair of edges. In an embodiment, the patient-facing wall has a substantially convex formation extending between the pair of opposite edges. In an embodiment, the non-patient-facing wall has a substantially flat or planar formation extending between the pair of opposite edges.

The non-delivery side member may be configured to be partially sunken or recessed into a patient's face so as to facilitate formation of an undisrupted seal between a patient mask and the patient's face. The non-delivery side member may be slightly recessed into the patient's skin such the non-patient-facing wall is approximately flush or aligned with the patient's skin. The non-patient-facing wall may thereby form a substantially continuous surface with the patient's skin and onto which the face mask may form a substantially undisrupted seal.

The above discussion contains various embodiments and examples of the gas sampling interface being provided to a non-delivery side member. However, as noted in earlier discussion, a patient interface according to the first aspect of the disclosure may also be configured with the gases sampling interface provided to the gases delivery side member. The gases sampling interface may therefore be located at, on, within or be otherwise physically associated with the gases sampling interface.

In an embodiment, the gas sampling interface is provided to the gases delivery side member the gases delivery side member comprising a delivery inlet at one end to receive the apparatus gas flow and the gases delivery side member comprising a patient-facing wall and a non-patient-facing wall. In an embodiment, the sampling inlet is proximate to the delivery outlet and the sampling outlet is proximate to the delivery inlet.

In an embodiment, the sampling conduit comprises a sampling lumen for the patient gas flow and the gases delivery side member comprising a gases delivery lumen for the apparatus gas flow. In an embodiment, the patient interface comprises a single sampling conduit. According to an embodiment, all of the patient gas flow that is communicated from the sampling inlet to the sampling outlet is communicated through the single sampling conduit. The physical association between the sampling lumen and the gases delivery lumen can be configured in a variety of ways.

In an embodiment, the sampling conduit is integrated with the gases delivery side member.

In an embodiment, the sampling conduit extends through the gases delivery lumen.

In an embodiment, a portion of the sampling conduit is free to move within the gases delivery lumen.

In an embodiment, the sampling conduit extends through the collapsible portion and the sampling conduit having a cross-section configured to minimise obstruction of apparatus gas flow and to minimise interference with moving of the collapsible portion to the collapsed configuration.

In an embodiment, the cross-section of the sampling conduit has a geometry configured to facilitate occlusion of the gases delivery lumen when the collapsible portion is in the collapsed configuration.

In an embodiment, the sampling conduit has a cross-section with a curved exterior surface configured for a wall the collapsible portion to bend or fold around the curved exterior surface.

In an embodiment, the sampling conduit has a cross-sectional area that is less than a cross-sectional area of the gases delivery lumen.

In an embodiment, the gases delivery side member extends through the sampling lumen.

In an embodiment, the sampling conduit comprises a sleeve that surrounds the gases delivery side member.

In an embodiment, the sampling inlet comprises a funnel portion at an end of the sleeve proximate to the delivery outlet, the funnel portion configured to receive patient gas flow from the nose and/or mouth.

In an embodiment, the funnel portion is configured to receive a portion of the apparatus gas flow.

In an embodiment, the gases delivery lumen and the sampling lumen are substantially concentric.

In an embodiment, the gases delivery lumen and the sampling lumen are substantially co-axial.

In an embodiment, the gases delivery lumen and the sampling lumen have parallel longitudinal axes.

In an embodiment, the gases delivery lumen and sampling lumen are integrally formed within the gases delivery side member and spaced apart from one another.

In an embodiment, the sampling lumen is formed within a wall of the gases delivery side member which surrounds the gases delivery lumen.

In an embodiment, the sampling conduit is integrated with the gases delivery side member. The sampling conduit may be integrally formed with the gases delivery side member. The sampling lumen and gases delivery lumen may therefore also be integrally formed. For example, the sampling lumen and gases delivery lumen may be simultaneously moulded during manufacture of the delivery side member.

In an embodiment, the sampling conduit comprises a sampling lumen with an elongate cross-sectional shape. The cross-section of the sampling lumen could be circular.

In an embodiment, the sampling conduit extends through the gases delivery lumen. The sampling lumen (inside of the sampling conduit) may therefore also be located within the gases delivery lumen. In an embodiment, a portion of the sampling conduit is free to move within the gases delivery lumen. For example, the sampling conduit may be fed through the gases delivery lumen so as to be loosely contained by the gases delivery lumen and capable of movement therein. Alternatively, the sampling conduit may extend through the gases delivery lumen but be fixed in a particular position therein. For example, by one or more ribs or webs which connect an outer surface of the sampling conduit to an peripheral surface of the gases delivery lumen. The sampling conduit may be fixed at certain portions relative to the gases delivery lumen but has other portions permitted to move within the gases delivery lumen.

In an embodiment, the sampling conduit extends through the collapsible portion and the sampling conduit has a cross-section configured to minimise obstruction of apparatus gas flow and to minimise interference with moving of the collapsible portion to the collapsed configuration. The sampling conduit may have a configuration which facilitates a reduction in apparatus gas flow through the collapsible portion when the collapsible portion is moved to the collapsed configuration. The cross-section of the sampling conduit can have a geometry configured to facilitate occlusion of the gases delivery lumen when the collapsible portion is in the collapsed configuration. For example, the sampling conduit can have a cross-section with a curved exterior surface configured for a wall portion of the collapsible portion to bend or fold around the curved exterior surface.

The sampling conduit may be configured so to facilitate formation of a seal around the sampling conduit which restricts the flow of apparatus gases through the collapsible portion. For example, a seal between an exterior surface of the sampling conduit and an interior surface of the collapsible portion. The sampling conduit may have a substantially curved cross-section such as a circular cross-section. The sampling conduit may have a cross-section without angular edges. The sampling conduit may therefore be configured to minimise or avoid interference with the collapsed configuration of the collapsible portion. That is, minimise or avoid interference with the reduction of stopping of apparatus gas flow when the collapsible portion is moved to the collapsed configuration.

The sampling conduit may be configured so as not to obstruct gas flow through the collapsible portion when the collapsible portion is in the open configuration. For example, an exterior surface of the sampling conduit may have a substantially smooth configuration (for example, a curved configuration) so as not to cause a choke point, blockage or other obstruction in the apparatus gas flow.

The sampling conduit may be configured in its size to minimise interference with the apparatus gas flow through the gases delivery lumen. The sampling conduit may have a cross-sectional area that is less than a cross-sectional area of the gases delivery lumen. In an embodiment, the sampling conduit has a width that is less than a width of the collapsible portion. A flow volume required of the gases delivery lumen may typically be higher than a flow volume required of the sampling lumen and therefore the gases delivery lumen may have a cross-sectional area that is larger than the cross-sectional area of the sampling lumen. For example, in some embodiments, a sampling flow rate of 40-500 mL/min of patient gases are provided through the sampling conduit. The sampling lumen may therefore be configured in its cross-section to facilitate a flow of approximately 40-500 mL/min. The gases delivery lumen may be configured for a significantly greater flow rate. In a particular embodiment, in the normally open configuration, the gases delivery interface is configured to allow an apparatus gas flow rate of about 20 L/min to 90 L/min through the apparatus gases flow path. In another particular embodiment, in the normally open configuration, the gases delivery interface is configured to allow an apparatus gas flow rate of between 5-70 L/min through the apparatus gases flow path.

In a particular embodiment, in the collapsed configuration, the patient interface is configured to allow an apparatus gas flow rate through the apparatus gases flow path that is at least 20 times greater than a patient gas flow rate through the gas sampling interface. For example, a flow rate through the gas sampling interface may be less than about 500 ml/min and a flow rate through the gases delivery interface when in the closed configuration may be less than about 10 L/min. In a particular embodiment, in the collapsed configuration, the gases delivery interface is configured to allow an apparatus gas flow rate of less than about 10 L/min through the apparatus gases flow path and the gas sampling interface is configured to allow a patient gas flow rate of less than about 500 mL/min, optionally about 40 mL/min to about 500 mL/min.

In an alternative embodiment, the gases delivery side member extends through the sampling lumen. For example, the gases delivery lumen may be provided in a gases delivery conduit which extends through the sampling conduit. In this instance, the sampling conduit may itself provide the gases delivery side member. Alternatively, the sampling conduit (with the gases delivery conduit extended therethrough) may be attached to or extend through the gases delivery side member.

In an embodiment, the sampling conduit comprises a sleeve that surrounds the gases delivery side member. In an embodiment, the sampling inlet is provided by a funnel portion at an end of the sleeve proximate to the delivery outlet, the funnel portion configured to receive patient gas flow from the nose and/or mouth. The funnel portion may include an opening which comprises the sampling inlet. The funnel portion could comprise a flared or enlarged portion of the sleeve. The funnel could have various configurations, for example a frustoconical or elongated-frustoconical or oval-frustoconical in configuration. In an embodiment, the funnel portion is configured to receive a portion of the apparatus gas flow.

In an embodiment, the gases delivery lumen and the sampling lumen are substantially concentric. For example, one of the gases delivery lumen and the sampling lumen extends through the other of the gases delivery lumen and the sampling lumen such that each shares a common central axis and (in cross-section) each shares a common central point. The gases delivery lumen and the sampling lumen may be substantially co-axial.

In an embodiment, the gases delivery lumen and the sampling lumen have parallel longitudinal axes. This configuration could be provided in a number of ways. In a first example, one of the gases delivery lumen and the sampling lumen may extend through the other of the gases delivery lumen or the sampling lumen. In a second example, the gases delivery lumen and the sampling lumen may extend alongside (and externally of) one another. In a third example, the gases delivery lumen and the sampling lumen are both formed internally within the gases delivery side member but also have parallel longitudinal axes.

In an embodiment, the gases delivery lumen and sampling lumen are integrally formed within the gases delivery side member and are spaced apart from one another. For example, spaced apart by an internal portion of the gases delivery side member. In an embodiment, the sampling lumen is formed within a wall of the gases delivery side member which surrounds the gases delivery lumen. In an embodiment, the gases delivery lumen is formed within a wall of the gases delivery side member which surrounds the sampling lumen. The wall-formed lumen (which could be either the sampling lumen or the gases delivery lumen) within the wall of the gases delivery side member could be localised to one side of wall-surrounded lumen. Alternatively, the wall-formed lumen could partially or completely surround the wall-surrounded lumen.

Some of the above embodiments relate to the sampling lumen and gases delivery lumen being each contained within an external surface of the gases delivery member. In an alternative embodiment, the sampling conduit extends alongside an external surface of the gases delivery side member. For example, the gases delivery lumen may be provided inside the gases delivery side member and the sampling conduit is located outside of the gases delivery side member. In an embodiment, the sampling conduit is connected with an external surface of the gases delivery side member.

In an embodiment, the sampling conduit integrally is connected with an external surface of the gases delivery side member.

In an embodiment, the sampling conduit is integrally connected with the external surface via a connection web and the sampling conduit is spaced apart from the gases delivery side member by a width of the connection web.

In an embodiment, the collapsible portion has an elongate cross-section which includes a pair of longitudinal sides extending between a pair of ends and wherein the connection web extends between the sampling conduit and one of the ends of the collapsible portion.

In an embodiment, the patient interface further comprises an accessory located at or proximate the collapsible portion and configured to facilitate the collapsible portion moving to the collapsed configuration.

In an embodiment, the accessory comprising a rigid member extending along the patient-facing wall of the gases delivery side member and is configured to provide a reaction force to a load applied to the collapsible portion.

In an embodiment, the accessory comprises a portion extending along the non-patient-facing wall of the gases delivery side member and configured to move towards the patient in response to a load applied to the accessory.

In an embodiment, the accessory comprises a conduit connector for connecting a portion of the sampling conduit to the accessory.

In an embodiment, the sampling conduit extends through the accessory.

In an embodiment, the accessory comprises pair of spaced apart openings comprising a patient gas inlet port configured for location proximate the delivery outlet and a patient gas outlet port configured for location proximate the delivery inlet, the sampling conduit extending internally through the accessory between the patient gas inlet and outlet.

In an embodiment the patient interface comprises a rigid gas path connector connectable with the delivery inlet and the patient interface further comprising a conduit connector for removably connecting a portion of the sampling conduit to the gas path connector.

In an embodiment, the conduit connector comprises a first attachment configuration comprising a pair of resilient arms configured for removable attachment to the gas path connector and a second attachment configuration configured for removable attachment to the sampling conduit.

In an embodiment, the second attachment configuration comprises a pair of hooks defining a pair of recesses corresponding to an external diameter of the sampling conduit and configured to receive and retain the sampling conduit.

In an embodiment, the sampling conduit is connected to both the conduit connector of the accessory and the conduit connector in connection with gas path connector.

In an embodiment, the gases delivery side member comprising a channel configured to receive the sampling conduit and permitting removable attachment between the sampling conduit and the gases delivery side member.

In an embodiment, the channel is located in the patient-facing wall of the gases delivery side member.

In an embodiment, the channel is located in the non-patient-facing wall of the gases delivery side member.

In an embodiment, the sampling conduit comprising a sampling lumen and the sampling conduit configured to retain the shape of the sampling lumen in response to the collapsing force applied to the collapsible portion.

The sampling conduit may be integrally connected with the external surface of the gases delivery side member. For example, the sampling conduit may be integrally connected with the external surface via a connection web and the sampling conduit is spaced apart from the gases delivery side member by a width of the connection web.

In an embodiment, the collapsible portion has an elongate cross-section which comprises a pair of longitudinal sides extending between a pair of ends and wherein the connection web extends between the sampling conduit and one of the ends of the collapsible portion.

The above-noted spacing between the sampling conduit and the gases delivery side member may advantageously facilitate movement of the collapsible portion to the collapsed configuration whilst not affecting the sampling conduit. For example, the sampling conduit may be spaced from the gases delivery side member so that flattening or compression of the collapsible portion during application of a patient face mask does not impact, deform, collapse or otherwise interfere with the patient gas flow through the sampling conduit.

In some embodiments, the patient interface further comprises an accessory located at or proximate the collapsible portion and configured to facilitate the collapsible portion moving to the collapsed configuration. Various examples of a suitable accessory are described in Applicant's U.S. Provisional application 63/362,486filed 4 Apr. 2022. It will be appreciated that an accessory described in U.S. provisional application 63/362,486 may be used with a patient interface according to the present disclosure. For example, an accessory disclosed in U.S. 63/362,486 may be used in conjunction with a gas sampling interface including a gas sampling conduit. A sampling conduit could be integrated with an accessory according to 63/362,486. In an embodiment, the accessory comprises a rigid member extending along the patient-facing wall of the gases delivery side member and configured to provide a reaction force to a load applied to the collapsible portion. In an embodiment, the accessory comprises a portion extending along the non-patient-facing wall of the gases delivery side member and configured to move towards the patient in response to a load applied to the accessory. The accessory may operate to enhance, focus, amplify or supplement a collapsing force applied to the collapsible portion.

The sampling conduit may be located external of the accessory but may be associated with the accessory. For example, in an embodiment the accessory comprises a conduit connector for connecting a portion of the sampling conduit to the accessory. Alternatively, in an embodiment the sampling conduit extends through the accessory. The sampling conduit may comprise a tube extending through a passageway integrally formed in the accessory. Alternatively, the sampling conduit may be provided by a passageway integrally formed in the accessory.

In an embodiment, the accessory includes pair of spaced apart openings comprising a patient gas inlet port configured for location proximate the delivery outlet and a patient gas outlet port configured for location proximate the delivery inlet, the sampling conduit extending internally through the accessory between the patient gas inlet and outlet.

In an embodiment, the patient interface further includes a rigid gas path connector connectable with the delivery inlet and the patient interface further comprising a conduit connector for removably connecting a portion of the sampling conduit to the gas path connector. The gas path connector may be configured for connecting the gases delivery side member to a flow supply conduit, for example a conduit connected to the apparatus supplying the apparatus gas flow. The gas path connector may include a connection configuration for connecting the gas path connector to a headstrap.

In an embodiment, the conduit connector in connection with the gas path connector includes a first attachment configuration comprising a pair of resilient arms configured for removable attachment to the gas path connector and a second attachment configuration configured for removable attachment to the sampling conduit. In a particular embodiment, the second attachment configuration comprises a pair of hooks defining a pair of recesses corresponding to an external diameter of the sampling conduit and configured to receive and retain the sampling conduit.

The above-noted conduit connector of the accessory may cooperate with the above-noted conduit connector in connection with the gas path connector. For example, in an embodiment, the sampling conduit is connected to both the conduit connector of the accessory and the conduit connector that is connected to the gas path connector.

As noted, the sampling conduit according to the first aspect of the disclosure is configured to remain open to maintain fluid communication between the sampling inlet and sampling outlet when the collapsible portion is moved to the collapsed configuration. This can be achieved in a variety of ways some of which were noted in the foregoing. For example, the sampling conduit being connected to the gases delivery member but spaced therefrom by a connection web such that the sampling conduit is not contacted or affected by a collapsing force applied to the collapsible portion. Various other configurations which maintain the sampling conduit in an open state are envisaged.

For example, in an alternative configuration, the sampling conduit can be located with respect to the collapsible portion such that the sampling conduit receives or is exposed to or influenced by the collapsing force applied to the collapsible portion. This could occur directly (for example a face mask directly contacting and pressing onto the sampling conduit) or could occur indirectly (for example a face mask force being applied to the collapsible portion and transmitted through the collapsible portion onto the sampling conduit). In either of these or other scenarios, the sampling conduit can be configured to remain open in response to direct or indirect application of the collapsing force to the sampling conduit.

For example, in an embodiment, the sampling conduit comprises a sampling lumen and the sampling conduit is configured to retain the shape of the sampling lumen in response to the collapsing force applied to the collapsible portion. In an embodiment, the sampling conduit is configured to be stiffer than the collapsible portion to maintain the shape of the sampling lumen upon application of the collapsing force. In a particular embodiment, the sampling conduit is formed of a material having sufficient material stiffness to retain the shape of the sampling lumen. In an embodiment, the gas sampling interface is formed of a different material than the collapsible portion. In an embodiment, the sampling conduit is formed of a different material than the collapsible portion. In an embodiment, the sampling conduit is formed of a material that has greater material stiffness than a material of the collapsible portion. In an embodiment, the gas sampling interface comprises silicone. In an embodiment, the collapsible portion comprises thermoplastic elastomer. In an embodiment, the gas sampling interface comprises silicone and the collapsible portion comprises thermoplastic elastomer.

In an embodiment, the sampling conduit is configured via geometric features to be stiffer than the collapsible portion. For example, the sampling conduit may have a thicker wall than the collapsible portion. The sampling conduit may comprise a wall of uniform (i.e. consistent) thickness whereas the collapsible portion may include thin-walled portions configured to facilitate bending or folding and thereby facilitate movement of the collapsible portion to the collapsed configuration. The sampling conduit may include an internal reinforcement structure such as struts or cross-members which help to resist closure of the sampling conduit.

According to a second aspect of the disclosure, there is provided a patient interface comprising:
a gases delivery interface configured to deliver an apparatus gas flow to a patient, the gases delivery interface comprising:
   a delivery outlet to deliver the apparatus gas flow to the patient; and
   a gases delivery side member extending from a side of the delivery outlet and comprising an apparatus gases flow path in fluid communication with the delivery outlet, the gases delivery side member comprising a collapsible portion movable upon application of a collapsing force from a normally open configuration to a collapsed configuration in which the apparatus gases flow path is reduced or closed and in order to reduce or stop the apparatus gas flow through the apparatus gases flow path, and the patient interface further comprising:
a gas sampling interface comprising:
   a sampling inlet configured to receive a patient gas flow at the patient;
   a sampling outlet configured for delivery of the patient gas flow away from the patient; and
   a sampling conduit in fluid communication with the sampling inlet and the sampling outlet, the gas sampling interface being provided to the gases delivery side member.

According to the second aspect of the disclosure, the gas sampling interface is therefore provided to the gases delivery side member. The various embodiments and features of the disclosure discussed above with respect to the first aspect of the disclosure will be understood as being applicable to the second aspect of the disclosure also, with the exception of the above-discussed embodiments which relate to the gases delivery interface being provided to the non-delivery side member.

In contrast to the first aspect of the disclosure, the sampling conduit of the second aspect of the disclosure is not necessarily configured to remain open when the collapsible portion is moved to the collapsed configuration. However, it is to be appreciated that the sampling conduit of the second aspect of the disclosure could be configured to remain open in the same manner as the first aspect of the disclosure.

The various embodiments and features of the disclosure discussed above with respect to the first aspect of the disclosure which relate to the sampling conduit remaining open when the collapsible portion is moved to the collapsed configuration may therefore also be applicable and/or implemented to the second aspect of the disclosure.

However, in an embodiment of the second aspect of the disclosure, the sampling conduit may be configured such that the patient gases flow can be selectively reduced or stopped. This may be desired in applications where a second respiratory support system (for example a face mask applied to a patient's face) also has gas sampling functionality and wherein a gas sampling interface on the collapsible cannula could introduce a leak to during use of the second respiratory support. Accordingly, in some situations, it may be desirable to stop patient gas flow through the gas sampling interface in order to not disrupt the secondary respiratory support system.

According to an embodiment of the second aspect of the disclosure, the sampling conduit is movable from a normally open configuration to a collapsed configuration in which the patient gas flow through the sampling conduit is reduced or stopped. The sampling conduit may have a configuration similar to that of the collapsible portion which is discussed in the foregoing and which facilitates closure, occlusion or restriction of the sampling conduit in response to a collapsing force. For example, a collapsing force applied to the collapsible portion by a patient face mask. The sampling conduit may be configured such that no patient gas flow occurs when in the collapsed configuration. Alternatively, the sampling conduit may be configured such that some residual patient gas flow still occurs through the sampling conduit when in the collapsed configuration. For example, one or more residual openings, channels or passageways may remain of a sampling lumen inside the sampling conduit when the sampling conduit is moved to the collapsed configuration and some residual patient gas flow may still flow through these residual openings, channels or passageways.

In an embodiment, the sampling conduit includes one or more side portions configured to move toward or against each other upon application of the collapsing force to reduce or stop the patient gas flow through the sampling conduit. The sampling conduit may comprise a material or geometry that is configured to facilitate movement of the side portions toward or against each other. In an embodiment, the sampling conduit moving from the open configuration to the closed configuration comprises bending or folding of one or more sides of the sampling conduit. The sampling conduit may include one or more thin-walled portions configured to provide folding lines at which the sampling conduit folds or bends upon application of the collapsing force. In an embodiment, the sampling conduit is resiliently deformable from the normally open configuration to the collapsed configuration.

The foregoing discussion relates to possible embodiments and features of the disclosure which relate to one or both of the first and second aspects of the disclosure. Various other possible embodiments and/or features of the disclosure may be applicable to both aspects of the disclosure and some of which are discussed below.

In an embodiment, the sampling inlet is located adjacent to the delivery outlet or within the delivery outlet. The sampling inlet may comprise a mouth scoop configured for location in front of the patient's mouth. The mouth scoop may comprise an opening configured to capture patient gases exhaled from the patient's mouth and/or nares. The mouth scoop may therefore form part of the sampling interface. In an embodiment, the mouth scoop is configured for removable attachment to the sampling conduit. In an embodiment the sampling inlet may be provided by the mouth scoop. For example, the mouth scoop may be configured for removable attachment to the sampling inlet and whereupon selective attachment of the mouth scoop to the sampling inlet, the sampling inlet is then provided by or at the mouth scoop. In an embodiment the mouth scoop may be non-removably attached to the sampling conduit and wherein the sampling inlet is provided by the mouth scoop.

The delivery outlet may comprise one or more nasal delivery prongs configured for location in one or both of the patient's nares. In an embodiment, the sampling inlet comprises a nasal and a mouth inlet.

In an embodiment, the sampling inlet is provided by a sampling nasal prong configured for insertion into a patient's nares. The sampling inlet could be provided by a pair of nasal prongs. The sampling conduit may extend beyond a distal end of the nasal delivery prongs such that the sampling nasal prong is configured to locate deeper in the patient's nares than the nasal delivery prongs.

The positioning of the sampling nasal prong relative to the nasal delivery prong could vary. In an embodiment, the sampling nasal prong extends through the nasal delivery prong. The sampling nasal prong may be located substantially centrally in the nasal delivery prong. For example, the sampling nasal prong and the nasal delivery prong may be concentric. Alternatively, the sampling nasal prong may be located externally of the nasal delivery prong and extending alongside the nasal delivery prong.

In an embodiment, the sampling conduit has a flexibly resilient support structure allowing the sampling conduit to be manipulated into a desired shape and/or allowing the sampling inlet to be located at a desired position. The support structure could comprise a wire located within the sampling conduit or associated with a wall of the sampling conduit. The wire may be formed of steel and may be flexible so as to allow selectively positioning of the wire into a desired shape or position.

In an alternative embodiment, the sampling conduit is provided without the above-noted flexibly resilient support structure. For example, the sampling conduit may be provided without a wire located within the sampling conduit. In an embodiment, the sampling conduit is provided with a single lumen and the single lumen is a sampling lumen for the patient gas flow. According to this embodiment, the sampling conduit does not include a support wire and therefore does not include any additional lumen for a support wire. In an embodiment, the sampling conduit has a homogenous material composition. For example, the sampling conduit may be formed only of a silicone or silicone-type material and not of a silicone material with an embedded support wire formed of a metallic or non-silicone material, as per the previously-mentioned alternative embodiment. The provision of a sampling interface with a single lumen that is the sampling lumen (i.e. without a flexibly resilient support wire accommodated in a wire lumen) may advantageously save material and reduce manufacturing complexity.

In an embodiment, the sampling conduit has an outer surface configured to seal against a mask cuff of a patient face mask. The sampling conduit may include a non-patient-facing surface which could be configured to seal against the mask cuff. The non-patient-facing surface may be provided with a stiffer configuration than the mask cuff such that the mask cuff is resiliently deformed around the non-patient-facing surface so as to form a seal between the mask cuff and the sampling conduit.

In an embodiment, the sampling inlet is located proximate the delivery outlet so as to be located within a cavity formed between a patient face mask and the patient's face during application of the face mask to the patient and wherein the sampling outlet is spaced from the delivery outlet so as to be outside of the cavity during the application of the mask to the patient. In this manner, a patient face mask applied to a patient's face will cover the sampling inlet but not the sampling outlet which is in (or available for) fluid communication with a respiratory gas monitor.

It will be appreciated from the above discussion that the present disclosure provides a patient interface configured to allowing sampling and monitoring of patient gases when the collapsible portion is moved to the collapsed configuration. For example, when a patient face mask is placed over the patient interface. The patient interface therefore allows for respiratory systems to be switched (for example, from a high-flow nasal canula to a face mask). It will also be appreciated from the above discussion that the patient interface can be configured so as not to disrupt (or to minimise disruption) of a mask seal placed against the patient's face.

Patient Interface and Accessory therefor

This disclosure also relates to a patient interface and an accessory therefor.

As discussed in the foregoing, the collapsible conduit includes a collapsible portion configured to collapse and restrict respiratory gas flow when, for example, a mask is placed onto a patient's face and a portion of the mask, e.g. the mask cuff, is overlaid across (and pressed onto) the collapsible portion. This allows a clinician to easily apply a non-invasive ventilation mask to the patient over the patient interface when needed and with a restriction of gas flow through the collapsed conduit

When a clinician wishes to provide manual ventilation using a bag and mask to a patient, the clinician may need to apply a considerable amount of pressure on the collapsible portion to cause sufficient collapse. Insufficient collapse may result in a residual gas flow through the collapsible portion.

An accessory according to this aspect of the disclosure may be provided in order to reduce residual flow and/or to otherwise improve the consistency or ease with which sufficient collapse is achieved.

The accessory may be configured in a variety of different ways to help facilitate or promote improved collapse of the collapsible portion. For example, the accessory may be configured to be located in a particular position with respect to the collapsible portion in order to promote sufficient collapse. Alternatively, or in addition, the accessory may include geometric features that provide a localised concentration of force on the collapsible portion to amplify a pressure applied to the collapsible portion. In another example, the accessory may be configured to amplify a force applied to the collapsible portion. Various embodiments of these and other concepts will be discussed below.

According to an aspect of the disclosure, there is provided an accessory for a patient interface configured to deliver respiratory gases to a patient via a gases delivery conduit which includes a collapsible portion, the accessory comprising: an attachment configuration configured to attach the accessory to the patient interface; and a contact portion configured to facilitate collapse of the collapsible portion at a collapse location in fixed relation to the attachment configuration, when a collapsing force is applied to the contact portion and/or to the collapsible portion.

The accessory may advantageously improve collapse consistency by promoting or otherwise facilitating collapse of the collapsible portion at a predetermined collapse location in fixed relation to the attachment configuration. This configuration may provide the user with control over the collapse location by altering the position of the accessory and the attachment configuration.

The accessory may include a platform, bar or plate locatable behind the collapsible portion (i.e. between the patient's face and a patient-facing side of the collapsible portion) to provide a rigid surface against which the collapsible portion is compressed when a collapsing force is applied to the collapsible portion. For example, the accessory may provide a backing plate configured to support the collapsible portion and provide a firm surface against which the collapsible portion is squeezed and compressed when a bag mask is applied to the patient's face.

The contact portion of the accessory may provide a surface against which the collapsible portion is pressed such that the collapsible portion is pinched or sandwiched between the mask cuff and the accessory. The contact portion may be configured to engage the collapsible portion in order to facilitate collapse of the collapsible portion. For example, the contact portion may be configured to contact the collapsible portion. The contact portion may be configured to deliver a force to the collapsible portion in order to cause or assist in collapsing the collapsible portion.

The contact portion may contact the patient interface at the collapsible portion. Alternatively, the contact portion may contact the patient interface at a location other than the collapsible portion for example, at a position on the gases delivery conduit that is upstream or downstream of the collapsible portion. In this instance, the contact portion may still facilitate collapse of the collapsible portion. For example, the contact portion may contact part of the gases delivery conduit adjacent to the collapsible portion and may provide a hinge or pivot point about which the gases delivery conduit may fold or kink to promote or induce collapse of the collapsible portion.

The contact portion may contact and support one or more parts of the gases delivery conduit adjacent to the collapsible portion and whereby at least a section of the collapsible portion is unsupported by the accessory. An unsupported section of the collapsible portion may span between a supported section of the collapsible portion or of the gases delivery conduit. The accessory may be configured to promote collapse at the unsupported section spaning between one or more supported sections.

According to a particular embodiment, the accessory includes a backing plate locatable, in use, between a patient-facing surface of the collapsible portion and the patient's face, the backing plate having a patient-facing surface and an opposite conduit-facing surface for facing the patient-facing surface of the collapsible portion.

The contact portion of the accessory may be configured in various ways. According to one embodiment, the contact portion comprising the conduit-facing surface of the backing plate. The conduit-facing surface of the backing plate may be configured to promote collapse of the collapsible portion at the collapse location. For example, the conduit-facing surface of the backing plate may comprise a concentration formation configured to concentrate force onto the patient-facing surface of the collapsible portion.

According to an embodiment, the concentration formation comprises at least one rib on the conduit-facing surface of the backing plate. The concentration formation may comprise a plurality of ribs. The concentration formation may comprise a serrated surface. The concentration formation could comprise other configurations such as a protrusion or a tapered protrusion. The concentration formation may be configured to contact the collapsible portion at a single position which corresponds to the collapse location.

In a particular embodiment, the backing plate is shaped with a pre-formed curve configured to conform with a contour of the gases delivery conduit and/or of the patient's face. The pre-formed curve may advantageously match or conform with a patient's face so as to direct or arrange the gases delivery conduit in a desired position with respect to the patient's face and/or to improve comfort for the patient.

The accessory may be provided in a variety of sizes to accommodate a variety of patient face sizes or shapes. For example, the accessory may be provided with a backing plate sized and/or shaped to accommodate a child's face and could also be provided with a backing plate sized and/or shaped to accommodate an adult's face. In this manner, a healthcare provider such as a nurse may select an appropriately sized accessory on the basis of characteristics of the patient's face.

In some embodiments of the disclosure, the accessory is not provided with a backing plate but may be provide with an alternative component. For example, the accessory may include a support member extending between the attachment configuration and the contact portion, the support member configured to support the contact portion in fixed relation to the attachment configuration.

In an embodiment, the support member may include a pre-formed curve to conform with a contour of the gases delivery conduit and/or the patient's face.

In an embodiment, the contact portion comprises a tapered configuration configured to concentrate force onto a patient-facing surface of the collapsible portion.

In an embodiment, the tapered configuration comprises a tapered rib located at a distal end of the support member.

In an embodiment, the tapered rib comprises an edge configured to concentrate force onto a patient-facing surface of the collapsible portion. The edge may comprise a linear or planar or straight edge. The edge may comprise a rounded or non-linear or non-planar edge.

In an embodiment, the edge is substantially transverse to a length of the collapsible portion.

In an embodiment, the contact portion comprises a first member locatable, in use, between a patient-facing surface of the collapsible portion and the patient's face; and a second member connected in movable association with the first member and configured to move toward the first member and facilitate collapse of the collapsible portion in response to application of the collapsing force. In use, the accessory may be fitted or otherwise located at or on the collapsible portion such that the collapsible portion is positioned between the first and second members. The movable association between the first and second members may be configured to squeeze, compress, sandwich, pinch or clamp the collapsible portion between the first and second members in order to promote collapse of the collapsible portion.

In an embodiment, the attachment configuration comprising an opening configured to receive the collapsible portion and to locate the collapse location of the collapsible portion between the first and second members. The first and second members may be configured to apply a clamping load at the collapse location of the collapsible portion upon application of the collapsing force.

In an embodiment, the second member includes an application surface configured to receive the collapsing force and whereby application of the collapsing force onto the application surface induces movement of the second member toward the first member. The application surface may comprise a portion of the second member located on a non-patient facing side (i.e. an outer side) of the collapsible portion. For example, a portion of the second member first contacted by a bag mask applied to a patient's face. The application surface may be located at or adjacent the collapse location or could be spaced therefrom, depending on the particular configuration and length of the second member.

In an embodiment, each of the first and second members include a proximal end, a distal end and a clamping portion located between the proximal and distal ends, wherein the proximal and distal ends are hinged together and, in use, the collapsible portion is locatable between the respective clamping portions.

The movable association between the first and second members could involve a variety of different configurations. In a particular embodiment, the first and second members are hingedly connected and, in use, the collapsible portion is locatable between the first and second members. The hinged connection could comprise a pin extending between corresponding openings in ends of the first and second members. Alternatively, the hinged connection could comprise any other suitable configuration. For example, the hinged connection could comprise a flexible hinge portion connecting the first and second members.

In an embodiment, the first member comprises a backing plate and the second member comprises a cantilever member hinged or pivoted to the backing plate. In use, the backing plate is located behind the collapsible portion i.e. between the patient's face and the patient-facing side of the collapsible portion. At least a portion of the second member may be positioned in front of the collapsible portion i.e. on a non-patient-facing side of the collapsible portion such that a bag mask will contact the second member when applied to the patient's face. The back plate and/or the cantilever member may include one or more ribs for concentrating force onto the collapsible portion. In use, the collapsible portion may be compressed or pinched between a rib of the backplate and a rib of the cantilever member. This configuration may advantageously provide a reliable collapse location and a localised concentration of force provided by the ribs may decrease the force required to achieve sufficient collapse of the collapsible portion.

The attachment configuration of the accessory may be provided in a variety of configurations and may be advantageously configured to allow selective positioning or adjustment of the accessory location in order to achieve a desired collapse location. According to an embodiment, positional adjustment of the attachment configuration allows for positional adjustment of the collapse location. The attachment configuration may be attachable at an attachment location on the patient interface and wherein the collapse location is predetermined by selection of the attachment location. In use, a user may select (for example, visually) a desired collapse location on the collapsible portion and position the attachment configuration accordingly so as to achieve the desired collapse location. This configuration may also advantageously provide a consistent collapse location relative to the attachment configuration. That is, the accessory may enable a user to accurately predict where collapse of the collapsible portion will occur upon application of the collapsing force.

According to a particular embodiment, the attachment configuration is attachable to the collapsible portion. The attachment configuration may be attachable to a part of the patient interface other than the collapsible portion. For example, the attachment configuration may be attachable to a portion of the gases delivery conduit upstream and/or downstream of the collapsible portion.

In an embodiment, the attachment configuration is attachable to a rigid part of the patient interface. In a particular embodiment, the rigid part of the patient interface is a rigid part of the gases delivery conduit upstream of the collapsible portion, with respect to a flow direction of respiratory gases in the gases delivery conduit.

In an embodiment, the contact portion is co-located with the collapse location. The contact portion may induce collapse of the collapsible portion at an interface between the contact portion and the collapsible portion. Accordingly, the user can advantageously anticipate a consistent collapse location based on the location of the contact portion.

In one embodiment, the and the collapse location are spaced from the attachment configuration. For example, the contact portion and attachment configuration may be located on generally opposite ends of the accessory such that the contact portion and the collapse location are spaced from the attachment configuration by approximately the length of the accessory.

In one embodiment, the contact portion and the collapse location are co-located with the attachment configuration. For example, the attachment configuration may be positioned generally at or adjacent the same position on the collapsible portion as the contact portion. The collapse location may therefore be at approximately the same location as the attachment configuration. Accordingly, the user may expect collapse to occur at the same location as the attachment configuration.

In an embodiment, the attachment configuration comprises at least one of a mount, clamp, coupling, connector, fastener, clip, snap-fit attachment or a recess.

In an embodiment, the attachment configuration is configured to attach or detach from the patent interface without disrupting a flow of respiratory gases to the patient through the gases delivery conduit. This embodiment may advantageously allow a user to fit or install the accessory or to remove the accessory while a patient is still receiving respiratory support through the patient interface.

In an embodiment, the attachment configuration comprises an opening configured to receive the collapsible portion and whereby, in use, the collapsible portion extends through the opening. The opening may comprise an aperture or passageway through a portion of the accessory. The opening may comprise a recess, trough, groove or a cut-away portion. The collapsible portion may, in use, be surrounded by the opening for example, where the opening comprises an aperture. Alternatively, the collapsible portion may, in use, be only partially-surrounded by the opening for example, where the opening comprises a cut-away, trough, groove or recess.

In an embodiment, the attachment configuration is configured to maintain location of the contact portion relative to the collapsible portion.

In an embodiment, the collapse location is in fixed relation to the attachment configuration, regardless of where the collapsing force is applied on the collapsible portion and/or the contact portion.

In an embodiment, the contact portion is configured to cooperate with the collapsing force to provide a compressive force onto the collapsible portion at the collapse location.

In an embodiment, the contact portion is configured to provide force onto at least one discrete position at the collapse location. The discrete position may comprise a discrete point or edge or side of the collapsible portion.

In an embodiment, the contact portion is configured to provide force onto a plurality of discrete positions at the collapse location.

In an embodiment, the contact portion is configured to provide a reaction force onto the collapsible portion in response to application of the collapsing force. The reaction force may act on the collapsible portion in an opposite direction to the direction of the collapsing force. In an embodiment, the contact portion applies a reaction force onto the collapsible portion in a direction away from the patient's face.

In an embodiment, the collapsing force is an external force applied to the contact portion and/or the collapsible portion. In an embodiment, the collapsing force is an external force applied by a cuff of a patient mask.

In an embodiment, the accessory comprises a plurality of contact portions. For example, the accessory may include a pair of spaced apart contact portions configured to locate the collapsible portion in between the pair of contact portions. Alternatively, the accessory may include a series of contact portions comprise a plurality of tapered protrusions configured to produce a plurality of collapse locations in the collapsible portion.

In an embodiment, the contact portion is configured to provide a force to the collapsible portion when a mask is applied to the patient and whereby a cuff of the mask overlies the collapsible portion.

In an embodiment, the contact portion has a contact surface configured to contact the collapsible portion and to concentrate the collapsing force at an interface of the contact surface and the collapsible portion. In an embodiment, the contact surface has a relatively small area configured to concentrate the collapsing force at the interface. According to a particular embodiment, the contact surface is located at the tip of a tapered protrusion. The collapsing force applied to the collapsible portion and/or to the contact portion may be concentrated onto the collapse location by the tip of the tapered protrusion. According to a particular embodiment, the tapered protrusion comprises a tapered rib.

According to a particular embodiment, the collapse of the collapsible portion provides a reduced flow rate of respiratory gases through the patient interface.

According to another aspect of the disclosure, there is provided an accessory for a patient interface configured to deliver respiratory gases to a patient via a gases delivery conduit which includes a collapsible portion, the accessory comprising: an attachment configuration configured to attach the accessory to the patient interface; a contact portion configured to contact and facilitate collapse of the collapsible portion when a collapsing force is applied to the contact portion and/or to the collapsible portion; and an indicator configured to locate the application of the collapsing force.

This aspect of the disclosure may advantageously facilitate effective positioning of the collapsing force. For example, the indicator may enable more accurate and effective positioning of the bag mask onto the collapsible portion and/or positioning of the accessory with respect to the collapsible portion. The indicator may indicate to a user an optimum application site for the collapsing force so as to achieve optimum collapse of the collapsible portion.

According to an embodiment, the indicator comprising a visual indicator. According to a particular embodiment, the visual indicator comprises coloured indicia.

In an embodiment, the visual indicator comprises indicia of symbols or

markings.

In an embodiment, the visual indicator is configured to be visualised through a transparent or translucent part of the collapsible portion.

In an embodiment, the indicator comprises a tactile indicator.

In an embodiment, the tactile indicator comprises a tactile formation in a surface of the accessory. In an embodiment, the tactile indicator comprising at least one of a ribs, bar, indentation, depression, rebate, notch, cavity channel, slit, groove, opening, protrusions, bump or raised portion.

In an embodiment, the accessory includes a patient-facing surface and an opposing non-patient-facing surface and the indicator is located on the non-patient-facing surface. According to a particular embodiment, the indicator is located on the contact portion.

In an embodiment, the indicator is configured to provide a location guide for a user to apply the collapsing force onto the collapsible portion or onto the contact portion.

According to another aspect of the disclosure, there is provided an accessory for a patient interface configured to deliver respiratory gases to a patient via a gases delivery conduit which includes a collapsible portion, the accessory comprising: an attachment configuration configured to attach the accessory to the patient interface; and a contact portion configured to deliver a collapsing load to the collapsible portion when an external load is applied to the contact portion and/or to the collapsible portion, the accessory configured to amplify the external load such that the force and/or pressure of the collapsing load delivered to the collapsible portion is higher than the force and/or pressure of the applied external load.

In an embodiment, the accessory is configured to deliver a higher pressure to the collapsible portion than a pressure of the applied external load.

In an embodiment, the contact portion has a contact area configured to deliver the collapsing load at an interface between the contact portion and the collapsible portion. According to a particular embodiment, the contact area of the contact portion is relatively small. For example, the contact area may be smaller than an application area of the contact portion or collapsible portion onto which the external load is applied. It will be appreciated that an equivalent force applied to a smaller area results in an increase in the pressure applied to that area. Accordingly, when a collapsing load of a particular force and pressure is applied to the application area and resulting in an equivalent force delivered to the collapsible portion at the contact area, the pressure at the contact area is increased by virtue of the contact area being smaller than the application area.

In an embodiment, the accessory is configured to deliver a higher force to the collapsible portion than a force of the applied external load. In an embodiment, the accessory is configured to amplify the force of the applied external load by a predetermined multiple. According to an embodiment, the predetermined multiple is greater than 1. According to an embodiment, the predetermined multiple is between 1-20. According to an embodiment, the predetermined multiple is between 5-20. According to an embodiment, the predetermined multiple is between 5-15.

In an embodiment, the applied external load is the force or pressure of a bag mask being applied to the collapsible portion and the accessory is configured to deliver a force and/or pressure to the collapsible portion that is larger than the force and/or pressure at which the bag mask is applied.

In an embodiment, the lever mechanism includes a pivot and the lever mechanism is configured to produce a turning moment about the pivot in response to the applied external load. In an embodiment, the lever mechanism may be configured to receive the force of the applied external load at a first portion of the lever mechanism and to deliver an amplified force to the collapsible portion at a second portion of the lever mechanism which is spaced apart from the first portion of the lever mechanism. In an embodiment, the second portion of the lever mechanism being positioned closer to the pivot than the first portion of the lever mechanism.

In an embodiment, the accessory comprises a lever mechanism configured to amplify the force of the applied external load. The lever mechanism may comprise a second-class lever configuration. That is, a configuration where the load delivered to the collapsible portion is positioned between a fulcrum and the force applied to the lever mechanism.

In an embodiment, the second-class lever configuration comprises a pair of clamping arms pivotally connected at a fulcrum, the pair of clamping arms providing a pair of opposed clamping surfaces positioned between the fulcrum and a distal end of each clamping arm and a clamping region provided between the pair of opposed clamping surfaces configured to receive the collapsible portion.

In an embodiment, the accessory is configured for the external load to be applied to at least one of the clamping arms in a position at or adjacent the distal end of the clamping arm.

In an embodiment, a distance between the delivered collapsing load to the fulcrum is less than a distance between the applied external load to the fulcrum. It will be appreciated that the force of the applied external load creates a torque or moment in the clamping arm about the fulcrum. The moment will induce a larger force at positions along the clamping arm that are closer to the fulcrum. In this manner, the force delivered to the collapsible portion at its position closer to the fulcrum than the applied external force will be an amplification of the applied external force. The multiple of amplification may be predetermined by the fulcrum-to-delivered load distance and the fulcrum-to-applied load distance. By way of example, clamping surfaces positioned 1 cm from the fulcrum and the applied load positioned 3 cm from the fulcrum will result in a force amplification multiple of 3.

The accessory may be configured to amplify both force and pressure of the applied external load or may be configured to amplify just one of force or pressure. For example, the accessory may be configured to delivery approximately the same force to the collapsible portion as was applied by the external load but over a smaller area, resulting in an increase in pressure. In other words, the accessory may concentrate the same force over a smaller area to increase the pressure of the collapsing load applied to the collapsible portion.

According to another aspect of the disclosure there is provided an accessory for a patient interface configured to deliver respiratory gases to a patient via a gases delivery conduit which includes a collapsible portion, the accessory comprising: an attachment portion configured to attach the accessory to the patient interface; and a support locatable, in use, between the gases delivery conduit and the patient's face and configured to facilitate collapse of the collapsible portion when a collapsing force is applied to the collapsible portion.

In an embodiment, the support comprises a rigid support. The support may include a rigid surface configured to provide a resisting force to a patient-facing side of the collapsible portion in response to application of the collapsing force to a non-patient-facing side of the collapsible portion. In a particular embodiment, the support is rigid yet has some resilient flexibility.

In an embodiment, the support is shaped to conform to a contour of the collapsible portion and/or the patient's face. The support may be shaped with a curve. In an embodiment, the support includes a pre-formed curve configured to conform to a contour of the collapsible portion and/or the patient's face.

According to an embodiment, the attachment portion is in fixed relation to the support. For example, the support may be rigidly connected to or integrally formed with the attachment portion. In this manner, the position of the support may be adjusted or determined based on the position of the attachment portion.

In an embodiment, the attachment portion comprises at least one of a mount, clamp, coupling, connector, fastener, clip, snap-fit attachment or a recess. The attachment portion could comprise any suitable means of attachment and it will be appreciated that other forms of attachment may be suitable for use as the attachment portion. According to a particular embodiment, the attachment portion comprises a clip in fixed relation to the support. The clip may be formed of a rigid material. The clip may be formed of a resilient material. The clip may be formed of a deformable material.

In an embodiment, the clip is configured to provide a removable snap-fit connection to a rigid part of the patient interface. In one embodiment, the clip is configured to provide a connection to a rigid part of gases delivery conduit located upstream of the collapsible portion, with respect to a flow direction of gases in the gases delivery conduit.

In an embodiment, the clip comprises a C-shaped clip or U-shaped clip. For example, the clip could comprise a resiliently flexible C-shaped clip configured to snap-fit onto a rigid part of the gases delivery conduit. In an embodiment, the support extends from the clip. In an embodiment, the clip is configured to connect to a rigid part of the gases delivery conduit at a position upstream of the collapsible portion and the support extends from the clip in a downstream direction between the patient's face and the collapsible portion.

In an embodiment, the support comprises a backing plate. The backing plate may be configured to be located, in use, such that a longitudinal axis of the backing plate is approximately parallel with a longitudinal axis of the collapsible portion. The backing plate may have a width direction perpendicular to the longitudinal axis of the backing plate and the backing plate has a width along the width direction which is at least equal to a width of the collapsible portion. In an embodiment, the backing plate has a width that is larger than a width of the collapsible portion. According to an embodiment, the backing plate has a substantially planar configuration.

In one embodiment, the support includes a proximal end, a distal end and a curved portion located intermediate of the proximal and distal ends. In an embodiment, the attachment configuration located at or adjacent to the proximal end of the support.

In an embodiment, the patient interface comprises a nasal cannula comprising one or more prongs configured for insertion into the patient's nasal passages and the support is sized such that the distal end of the backing plate is, in use, spaced from the one or more prongs.

In an embodiment, the support comprises a contact portion configured to contact a patient-facing surface of the gases delivery conduit. The contact portion may be configured to contact a patient-facing surface of the collapsible portion. In an embodiment, the contact portion is configured to contact a patient-facing surface of the gases delivery conduit upstream or downstream of the collapsible portion, with respect to a flow direction in the gases delivery conduit.

In an embodiment, the contact portion is configured to provide a space or cavity between the support and the patient-facing surface of the gases delivery conduit in a region upstream of the contact portion, with respect to a flow direction of respiratory gases in the gases delivery conduit.

In an embodiment, the accessory is configured to allow the collapsible portion to collapse into the space or cavity upon application of the collapsing force. In an embodiment, the accessory is configured to allow the collapsible portion to kink into the space or cavity upon application of the collapsing force. For example, the accessory may be configured to engage with the collapsible portion so as to produce a kink in the collapsible portion. The accessory may be configured to produce more than one kink in the collapsible portion. The accessory may be configured to collapse and/or kink the collapsible portion so as produce a tortuous path for the respiratory gases and reduce flow rate by increasing flow resistance through the tortuous path.

In an embodiment, the support includes an outwardly-facing surface configured to, in use, face the patient-facing surface of the collapsible portion and wherein the contact portion extends from the outwardly-facing surface of the support in an outward direction away from the patient's face.

In an embodiment, the support includes a patient-facing side for facing the patient's face and an outwardly-facing side for facing the patient-facing surface of the collapsible portion and a concentration formation located at the outwardly-facing side of the support for concentrating a resisting force onto the collapsible portion. In an embodiment, the resisting force occurs in response to application of the collapsing force. That is, the support resists movement of the collapsible portion toward the patient's face by applying a resisting force to the patient-facing side of the collapsible portion which is concentrated by the concentration formation. In an embodiment, the concentration formation is configured to increase the pressure of the resisting force.

In an embodiment, the contact portion comprises at least one of a rib, bump, bar, serration, protrusion or raised portion. In an embodiment, the contact portion is located at or adjacent a distal end of the support.

In an embodiment, the contact portion comprises one or more ribs. In an embodiment, the one or more ribs extend at least partially between a pair of opposing longitudinal edges of the support. In an embodiment, the one or more ribs are orientated parallel with a longitudinal axis of the support.

In an embodiment, the support includes a first rib and a second rib spaced apart from the first rib by a seat configured to receive the collapsible portion. In an embodiment, the first and second ribs extend along opposing raised edges of the support. In an embodiment, the opposing raised edges comprise a pair of opposite first and second longitudinal edges, the first rib located adjacent the first longitudinal edge the second rib located adjacent the second longitudinal edge.

According to a particular embodiment, the first rib is configured to contact an upper portion of the collapsible portion and the second rib is configured to contact a lower portion of the collapsible portion. For example, in use the support may be orientated such that the first rib is at a higher position than the second rib. The first rib may therefore make contact with the collapsible portion at a higher position on the collapsible portion than the second rib which makes contact with a lower position on the collapsible portion.

According to an embodiment, the first and second ribs are pivotally connected to locate the collapsible portion in the seat.

According to an embodiment, the first and second ribs are movable toward or away from one another and between an open configuration configured to receive the collapsible portion and a closed configuration configured to engage the collapsible portion between the first and second ribs.

In an embodiment, the first and second ribs are biased toward the open configuration and movable toward the closed configuration upon application of the collapsing force.

In an embodiment, the accessory is attachable to the patient interface with the attachment configuration positioned upstream of the support, with respect to a flow direction of respiratory gases in the gases delivery conduit.

In an embodiment, the support comprises a backing plate comprising a conduit-facing surface which comprises an elongate protrusion and the accessory comprising a pair of attachment portions comprising a pair of flanges extending from opposing sides of the backing plate, each flange comprising an opening configured for the collapsible portion to extend therethrough.

In an embodiment, the backing plate is configured to protect or shield the patient's face from the applied the collapsing force. For example, the backing plate may be rigidly connected to the patient interface via the attachment configuration such that the applied collapsing force is not transmitted to the patient's face.

In an embodiment, the attachment configuration comprises a clip and the backing plate extends from the clip. The clip may attach to a headstrap-pneumatic connector of the patient interface which acts as a support for the accessory. The backplate may be shaped with a curve along its length. The curve may allow for a substantial portion of the backplate to seat between the patient's face and the collapsible portion. In an embodiment, the backplate is more rigid than the collapsible portion. The rigidity of the backplate may be achieved via the backplate material and/or via the geometric configuration of the backplate.

In an embodiment, the clip comprises a C-shaped clip. In an embodiment, the clip comprises a U-shaped clip. In an embodiment, the attachment configuration may comprise alternative attachment means such as adhesive, over moulding or integral moulding.

In an embodiment of the disclosure, the backplate provides a hard surface behind the collapse location of the collapsible portion. The hard surface of the backing plate thereby provides a resisting force against the applied collapsing load, for example the collapsing force applied by a bag mask. In this manner, the backing plate helps facilitate an optimum collapse of the collapsible portion.

The backing plate may be elongated or generally rectangular and may have a larger width than the collapsible portion. According to a particular embodiment the backing plate may be provided in a range of sizes to suit a range of patient face sizes or shapes. The backing plate could be customised to fit a particular patient's face.

According to an embodiment, the backing plate includes a visual or tactile indicator to assist a user in applying a force to collapse the collapsible portion. The backing plate may be configured to indicate an optimised collapse location. For example, the backing plate may include a visual or tactile indicator of the position of the contact portion. In particular, the indicator may indicator to a user where the contact portion is positioned underneath the collapsible portion so that the user can apply a load at a position on the collapsible portion which overlies the contact portion.

According to another aspect of the disclosure there is provided, an accessory for a patient interface configured to deliver respiratory gases to a patient via a gases delivery conduit which includes a collapsible portion, the accessory comprising: a clamping configuration comprising first and second clamping members configured to receive the collapsible portion between the first and second clamping members, the first clamping member locatable, in use, between a patient-facing surface of the collapsible portion and the patient's face, the second clamping member connected in movable association with the first clamping member and configured to move toward the first clamping member and facilitate collapse of the collapsible portion in response to application of a collapsing force.

In an embodiment, at least one of the first and second clamping members includes a concentration formation configured to concentrate force onto the collapsible portion.

In an embodiment, the concentration formation comprises one or more ribs.

In an embodiment, the first clamping member includes a first rib extending toward the second clamping member and the second clamping member includes a second rib extending toward the first clamping member.

In an embodiment, one of the first or second clamping members includes a rib extending toward the other of the first or second clamping members.

In an embodiment, the second clamping member is configured to overlie a non-patient-facing surface of the collapsible portion. That is, the second clamping member is configured to overlie an outer or outward-facing side of the collapsible portion which faces away from the patient's face. The said non-patient-facing surface of the collapsible portion may, in use, be the surface which is contacted by the applied collapsing force, for example contacted by the cuff of a bag mask applied to the patient's face.

In an embodiment, the second clamping member includes a non-patient-facing surface which defines an application surface configured to receive the applied collapsing force and whereby application of the collapsing force onto the application surface induces movement of the second clamping member toward the first clamping member.

In an embodiment, the application surface is located at or adjacent a distal end of the second clamping member.

In an embodiment, the first and second clamping members are pivotally connected. The first and second clamping members may be hingedly connected at a hinged connection. The hinged connection may comprise a multi-part hinge mechanism such as a pin engaged with apertures in the clamping members. Alternatively, the hinge connection may comprise an integrally formed hinge such as a flexible portion of material connecting the first and second clamping members. It will be appreciated that other pivotal or hinge configurations may be suitable.

In an embodiment, the first and second ribs are located between the hinged connection and a distal end of the clamping members.

In an embodiment, the first and second clamping members have a second-class lever configuration configured to amplify the applied collapsing force which is delivered to the collapsible portion. It will be appreciated that a second-class lever configuration is a lever configuration wherein the load delivered by the lever is positioned between a fulcrum and the force applied to the lever mechanism. According to this embodiment, the load delivered to the collapsible portion is positioned between a fulcrum (the pivotal or hinged connection) and the force applied to the lever mechanism.

In an embodiment, the second clamping member includes an opening configured for the collapsible portion to extend through.

In an embodiment, at least one of the clamping members is formed of a flexibly resilient or soft material.

In an embodiment, the accessory further includes an attachment configuration configured to attach the accessory to the patient interface. The attachment configuration may be connected to the first clamping member. In an embodiment, the attachment configuration comprises a C-shaped clip or U-shaped-clip configured to provide a snap-fit connection to a portion of the patient interface.

In an embodiment, the clamping configuration is movable between an open position configured to allow unrestricted flow within the collapsible portion and a clamped position configured to apply restriction to the flow within the collapsible portion. In an embodiment, the clamping configuration is biased toward the open position and movable toward the clamped position upon application of the collapsing force.

According to an embodiment, the first and second clamping members include respective clamping surfaces biased apart by a biasing configuration. The biasing configuration may comprise, for example, a spring or a flexibly resilient linkage or strut.

In an embodiment, the first and second clamping members are configured to create a series of pinch points in the collapsible portion when in the clamped configuration. The first and second clamping members may be configured to create a series of kinks in the collapsible portion. In an embodiment, the first and second clamping members are connected via at least one linkage movable with respect to at least one of the clamping members. In a particular embodiment, the clamping members Are connected via a plurality of linkages. In an embodiment, each linkage in the plurality of linkages is flexibly resilient.

In an embodiment, the accessory includes four flexibly resilient linkages extending between the clamping members and configured to maintain the clamping members in a normally spaced apart configuration. That is, the linkages are configured to urge the clamping members to the spaced apart configuration. The resilient flexibility of the linkages allows the clamping members to be moved together under a load, for example a compressive load such as a force applied by application of a bag mask to one of the clamping members. The linkages are configured to return the clamping members to their normally spaced apart configuration when the force is removed.

In an embodiment, the linkages are configured to flexibly deform upon application of the collapsing force and allow the clamping members to collapse toward one another. In an embodiment, the clamping members have a square or rectangular profile and the four linkages extend from each of the four corners of the clamping members. In an embodiment, the four corners of the clamping members are each connected by one of the linkages. In an embodiment, the linkages are oriented approximately parallel with one another when the clamping members are in the normally spaced apart configuration. In an alternative embodiment, the linkages may have a crossed configuration wherein one or more linkages cross over at least one of the other linkages.

In an embodiment, the linkages have a normally linear formation. In an embodiment, the linkages having a normally non-linear formation. In an embodiment, the linkages having a diamond-shaped configuration.

In an embodiment, the clamping members have a plate or planar configuration. In an embodiment, each clamping member comprises a bar. In an embodiment, each clamping member has a cylindrical formation.

According to another aspect of the disclosure, there is provided an accessory for a patient interface configured to deliver respiratory gases to a patient via a gases delivery conduit which includes a collapsible portion, the accessory comprising: a first portion configured to apply a force to collapse a first region of the collapsible portion; a second portion spaced apart from the first portion and with a fulcrum located between the first and second portions, wherein application of a collapsing force in a first direction to the first portion causes movement of the second portion in a second direction to collapse a second region of the collapsible portion.

In an embodiment, the first and second portions are angled with respect to one another. The first and second portions may be angled at an obtuse angle with respect to one another. In a particular embodiment, the first portion is located on a first lever arm and the second portion is located on a second lever arm and wherein each of the lever arms extend from the fulcrum.

According to an embodiment, the accessory is rigid such that the first lever arm, the second lever arm and the fulcrum are in fixed relation to one another.

In an embodiment, the fulcrum comprises a rigid corner at an intersection or convergence of the first and second lever arms.

In an embodiment, the first portion includes an opening configured to receive the collapsible portion therethrough.

In an embodiment, the accessory further includes a support locatable between the patient's face and the fulcrum.

In an embodiment, the support comprises a backing plate. The backing plate may include a rigid surface configured to act as a pivot surface for the fulcrum.

In an embodiment, the fulcrum comprises a pivot connected to the backing plate.

In an embodiment, the first direction is a direction toward the patient's face and the second direction is a direction away from the patient's face.

In an embodiment, the first and second portions are spaced unequally from the fulcrum. According to a particular embodiment, the second portion is spaced farther from the fulcrum than the first portion. As noted above, the first portion may be located on a first lever arm and the second portion may be located on a second lever arm. According to this embodiment, the second lever arm may be longer than the first lever arm, relative to the fulcrum. That is, the second lever arm may extend from the fulcrum farther than the first lever arm extends from the fulcrum.

According to an embodiment, the accessory may comprise a see-saw configuration wherein each of the first and second lever arms extend from opposite sides of the fulcrum. In an embodiment, the accessory may have a generally ‘V’ shape.

According to another aspect of the disclosure, there is provided, an accessory for a patient interface configured to deliver respiratory gases to a patient via a gases delivery conduit which includes a collapsible portion, the accessory comprising:

a gripping portion configured to apply a force in a direction away from the patient's face to facilitate collapse or folding of the collapsible portion.

This aspect of the disclosure may advantageously provide a relatively simple and reliable means by which to promote collapse of the collapsible portion by providing a gripping portion which allows a user to pull on the collapsible portion in a direction away from the patient's face. This action may occur simultaneously with a mask being applied to the patient's face and overlaid on the collapsible portion. In this instance, the collapsible portion would be pulled outwardly away from the patient's face by the gripping portion and simultaneously pushed inwardly toward the patient's face by the mask. This simultaneous and opposing forces may thereby promote improved collapse of the collapsible portion.

In an embodiment, the accessory comprises a connector configured to connect a tube of the gases delivery conduit to the collapsible portion. For example, the accessory may be integrally formed with a connector of the patient interface.

According to an alternative embodiment, the accessory further includes an attachment configuration to attach the accessory to the patient interface. For example, the accessory may be removably connectable to the patient interface.

It will be appreciated from the foregoing that the gripping portion may be integrally formed with the connector or may be discrete and selectively attachable to the connector.

In an embodiment, the attachment configuration comprises an opening, clip or recess. In an embodiment, the attachment configuration includes a C-shaped or U-shaped clip.

According to an embodiment, the attachment configuration is configured to attach to a rigid portion of the gases delivery conduit.

In an embodiment, the attachment configuration is configured to attach to a rigid connector located between a gas delivery tube and the collapsible portion.

In an embodiment, the gripping portion extends from the connector and is configured to allow digital application of a pulling force away from the patient's face.

In an embodiment, the gripping portion comprises a hook, loop, ring or strap.

In an embodiment, the gripping portion comprises a rigid hook or ring.

In an embodiment, the gripping portion comprises a flexible finger loop or finger strap.

The accessory of any one of the foregoing aspects of embodiments of the disclosure may be formed of at least one of thermoplastic elastomer, thermoset or thermoplastic elastomer or metal. The accessory may be formed of titanium, steel, copper or nitinol. The accessory may be formed of other metallic materials, polymers or ceramics. The accessory may be formed of polyethylene.

It will be appreciated that the force required to achieve a sufficient or desirable level of collapse of the collapsible portion may vary depending on various factors such as the resilience of the collapsible portion as well as the respiratory gas pressure passing through the collapsible portion. However, according to a particular embodiment, the collapsible portion is collapsible upon application of a collapsing force of greater than 5N and more particularly a collapsing force of greater than 7N. In another embodiment, the collapsible portion is collapsible upon application of a minimum collapsing force of between 5N to 30N.

The accessory in the foregoing discussion may be configured for use with a patient mask such as a bag mask. The accessory may be configured to configured to cooperate with the patient mask when overlaid across the collapsible portion or a portion of the accessory. The accessory may be configured to cooperate with the mask whereby the mask seals over the accessory and the patient interface to form a seal with the patient's face.

According to an embodiment, the accessory is configured to amplify, concentrate or increase a collapsing load applied by a cuff of the patient mask.

In an embodiment, the accessory is operable to create a tortuous flow path in the collapsible portion.

In an embodiment, the accessory is operable to increase flow path resistance in the collapsible portion.

According to an aspect of the disclosure, there is provided a respiratory system which includes an accessory as discussed in any of the above-noted aspects or embodiments.

According to another aspect of the disclosure, there is provided a patient interface which includes an accessory as discussed in any of the foregoing aspects or embodiments.

In an embodiment, the patient interface includes a gases delivery conduit for delivering respiratory gases to a patient, the gases delivery conduit comprising a collapsible portion configured to collapse and restrict or occlude flow through the collapsible portion upon a collapsing force being applied to the collapsible portion.

In an embodiment of the patient interface, the collapsible portion comprises a portion of conduit formed of a resilient material and is configured to collapse under application of a collapsing force applied from a mask overlaid across the collapsible portion. Said patient interface may include an accessory according to any one of the foregoing aspects or embodiments.

In an embodiment, the patient interface comprises a nasal cannula. The nasal cannula may comprise a non-sealing nasal canula. The nasal cannula may comprise a cannula body and prongs extending from the cannula body and configured to provide respiratory gases to the patients' nares.

According to another aspect of the disclosure, there is provided a respiratory system configured to deliver respiratory gases to a patient via a gases delivery conduit which includes a collapsible portion, the system comprising: a respiratory gases flow source; a patient interface comprising a gases delivery conduit which has a collapsible portion; and an accessory configured to reduce flow of respiratory gases through the collapsible portion to less than a threshold flow rate.

In an embodiment, the respiratory system further includes a humidifier.

In an embodiment, the respiratory system includes a heated inspiratory tube.

In an embodiment, the accessory of the respiratory system is configured to reduce the flow of respiratory gases in response to application of a collapsing force applied to the accessory and/or to the collapsible portion.

In an embodiment, the respiratory system is configured to provide flow of the respiratory gases at flow rates of at least 20 L/min. In an embodiment, the respiratory system is configured to provide flow of the respiratory gases at flow rates of 20-90 L/min. In an embodiment, the respiratory system is configured to provide flow of the respiratory gases at flow rates of 40-70 L/min.

In an embodiment of the respiratory system, the accessory is configured to reduce the flow rate from an initial flow of 40-70 L/min to less than the threshold flow rate. In an embodiment of the respiratory system, the accessory is configured to reduce the flow rate an initial flow of approximately 70 L/min to less than the threshold flow rate. In an embodiment, the threshold flow rate is less than 10 L/min.

According to an embodiment of the respiratory system, the collapsible portion is collapsible from an open configuration to a collapsed configuration, the open configuration allowing an unrestricted flow of respiratory gases through the collapsible portion and the collapsed configuration providing a restricted flow of respiratory gases through the collapsed portion.

In an embodiment of the respiratory system, the unrestricted flow of respiratory gases ranges between 40-70 L/min and the restricted flow is less than the threshold flow rate.

In an embodiment of the respiratory system, the accessory is provided according to any one of the above-discussed aspects of embodiments of an accessory for use with a patient interface.

According to another aspect of the disclosure, there is provided a respiratory system configured to deliver respiratory gases to a patient via a gases delivery conduit which includes a collapsible portion, the system comprising: a respiratory gases flow source; a patient interface comprising a gases delivery conduit which has a collapsible portion; and an accessory configured to reduce the cross-sectional area of the collapsible portion by a minimum multiple.

In an embodiment of the respiratory system, the accessory is configured to reduce the cross-sectional area of the collapsible portion in response to application of a collapsing force applied to the accessory and/or the collapsible portion.

In an embodiment of the respiratory system, the accessory is configured to facilitate collapse of the collapsible portion to reduce the cross-sectional area.

In an embodiment of the respiratory system, the minimum multiple is at least 90%. In an embodiment, the minimum multiple is at least 95%.

In an embodiment of the respiratory system, the respiratory system includes an accessory according to any one of the above discussed aspects or embodiments for an accessory for use with a patient interface.

According to another aspect of the disclosure, there is provided a patient interface configured to deliver respiratory gases to a patient via a gases delivery conduit which includes a collapsible portion, the patient interface comprising an accessory configured to facilitate of the collapsible portion when a collapsing force is applied to the accessory and/or to the collapsible portion.

In an embodiment of the patient interface, the accessory is non-removably connected or integrally formed with the patient interface.

In an embodiment of the patient interface, the accessory is integrally formed with a portion of the gases delivery conduit.

In an embodiment of the patient interface, the accessory is removably connected to the patient interface. In an embodiment of the patient interface, the accessory includes an attachment configuration configured to attach the accessory to the patient interface.

In an embodiment of the patient interface, the accessory is provided according to any of the above-discussed aspects or embodiments of an accessory for use with a patient interface.

### BRIEF DESCRIPTION OF DRAWINGS

Specific embodiments and modifications thereof will become apparent to those skilled in the art from the detailed description herein having reference to the figures that follow, of which:

FIG. 1 shows a respiratory support system;

FIG. 2 shows a patient wearing a patient interface;

FIG. 3 shows a patient wearing a patient interface (a first patient interface) and a face mask (a second patient interface);

FIG. 4 shows a cross-section of a portion of a patient interface or conduit;

FIG. 5 shows a typical airway of a patient;

FIG. 6 shows a patient wearing a patient interface and a gas sampling interface;

FIG. 7 shows a patient interface configured to deliver apparatus gases to a patient via a gases delivery side member which includes a collapsible portion;

FIG. 8 shows a cross-section of a non-collapsible portion of the gases delivery side member in FIG. 7;

FIGS. 9 and 10 show a cross-section of the collapsible portion of the gases delivery side member in FIG. 7;

FIG. 11 is a front perspective view of a patient interface according to an embodiment;

FIG. 12 is a front view of the FIG. 11 embodiment;

FIG. 13 is a cross-sectional view taken along the section A-A illustrated in FIG. 12;

FIG. 14 is a front view of the first embodiment patient interface of FIG. 11 shown with a seal of a patient face mask;

FIG. 15 is a cross-sectional view of a mask seal, the patient interface and a patient's face;

FIG. 16 is a rear perspective view of a patient interface according to an alternative embodiment;

FIG. 17 is a rear perspective view of a patient interface according to an alternative embodiment;

FIG. 18 is a front perspective view of a patient interface according to an alternative embodiment;

FIG. 19 is a front perspective view of a patient interface according to an alternative embodiment;

FIG. 20 is a front perspective view of a patient interface according to an alternative embodiment;

FIG. 21 is a front perspective view of a patient interface according to an alternative embodiment;

FIG. 22 is a front perspective view of a patient interface according to an alternative embodiment;

FIG. 23 is a front perspective view of a patient interface according to an alternative embodiment;

FIG. 24 is a front perspective view of a patient interface according to an alternative embodiment;

FIG. 25 is a front perspective view of a patient interface according to an alternative embodiment;

FIG. 26 is a rear perspective view of a patient interface according to an alternative embodiment;

FIG. 27 is a side perspective view of the patient interface of FIG. 7 and

showing a cross-section of the non-delivery side member;

FIGS. 28 and 29 show alternative configurations of a conduit-receiving channel provided to a non-delivery side member of a patient interface;

FIG. 30 is a rear perspective view of a patient interface according to an alternative embodiment;

FIG. 31 is a rear perspective view of a patient interface according to an alternative embodiment;

FIG. 32 is a cross-sectional view of a gases delivery side member of a patient interface according to an alternative embodiment;

FIG. 33 shows the FIG. 32 cross-section in a collapsed configuration and with the sampling lumen remaining open;

FIG. 34 shows the FIG. 32 cross-section in a collapsed configuration and with the sampling lumen also in a collapsed configuration;

FIG. 35 is a cross-sectional view of a gases delivery side member of a patient interface according to an alternative embodiment;

FIG. 36 shows the FIG. 35 cross-section in a collapsed configuration and with the sampling lumen remaining open;

FIG. 37 shows the FIG. 35 cross-section in a collapsed configuration and with the sampling lumen also in a collapsed configuration;

FIG. 38 is a cross-sectional view of a gases delivery side member of a patient interface according to an alternative embodiment;

FIG. 39 shows the FIG. 38 cross-section in a collapsed configuration and with the sampling lumen remaining open;

FIG. 40 shows the FIG. 38 cross-section in a collapsed configuration and with the sampling lumen also in a collapsed configuration;

FIG. 41 is a cross-sectional view of a gases delivery side member of a patient interface according to an alternative embodiment;

FIG. 42 shows the FIG. 41 cross-section in a collapsed configuration and with the sampling lumen remaining open;

FIG. 43 shows the FIG. 41 cross-section in a collapsed configuration and with the sampling lumen also in a collapsed configuration;

FIG. 44 is a cross-sectional view of a gases delivery side member of a patient interface according to an alternative embodiment;

FIG. 45 shows the FIG. 44 cross-section in a collapsed configuration and with the sampling lumen remaining open;

FIG. 46 shows the FIG. 44 cross-section in a collapsed configuration and with the sampling lumen also in a collapsed configuration;

FIG. 47 is a cross-sectional view of a gases delivery side member of a patient interface according to an alternative embodiment;

FIG. 48 shows the FIG. 47 cross-section in a collapsed configuration and with the sampling lumen remaining open;

FIG. 49 shows the FIG. 47 cross-section in a collapsed configuration and with the sampling lumen also in a collapsed configuration;

FIG. 50 is a front perspective view of a patient interface according to an alternative embodiment;

FIG. 51 is a front perspective view of a patient interface according to an alternative embodiment;

FIG. 52 is a rear view of a patient interface according to an alternative embodiment;

FIG. 53 is a rear view of a patient interface according to an alternative embodiment;

FIG. 54 is a view of a closer detail of a portion of FIG. 53;

FIG. 55 is a front perspective view of a patient interface according to an alternative embodiment;

FIG. 56 is a front perspective view of a patient interface according to an alternative embodiment;

FIG. 57 shows the gas path connector of FIG. 56;

FIG. 58 is a cross-sectional view of the sampling line of FIG. 56;

FIG. 59 shows an attachment clip for connection to the gas path connector of FIG. 57;

FIG. 60 is a front perspective view of a patient interface fitted with an accessory and according to an alternative embodiment;

FIG. 61 is a view of an alternative accessory for use with a patient interface;

FIG. 62 shows the accessory of FIG. 61 connected with the gas path connector of FIG. 57;

FIG. 63 is a front perspective view of a patient interface according to an alternative embodiment and in which the patient interface is fitted with the accessory of FIGS. 61 and 62;

FIGS. 64 and 65 are rear and front perspective views of an alternative accessory for use with a patient interface in an embodiment of the disclosure;

FIG. 66 shows an accessory according to an aspect of this disclosure for use with a patient interface which includes a collapsible portion.

FIG. 67 shows the accessory of FIG. 66 fitted to a patient interface;

FIGS. 68 and 69 are side-sectional views of an uncollapsed configuration and a collapsed configuration respectively, of the accessory of FIGS. 6 and 7 when in use with a patient interface;

FIG. 70 is a perspective view of an accessory according to another embodiment of the present disclosure;

FIG. 70A is a perspective view of an accessory according to another embodiment of the present disclosure;

FIGS. 71 and 72 are side-sectional views of an uncollapsed configuration and a collapsed configuration respectively, of the embodiment illustrated in FIG. 70 when in use with a patient interface;

FIG. 73 is a perspective view of an accessory according to another embodiment of the present disclosure;

FIG. 74 is a perspective view of an accessory according to another embodiment of the present disclosure;

FIG. 74A illustrates a cross-section of a collapsible conduit on a flat backing plate and in an uncollapsed configuration;

FIG. 74B illustrates a cross-section of the collapsible conduit of FIG. 74A in a collapsed configuration;

FIG. 74C illustrates a cross-section of a collapsible conduit in an uncollapsed configuration and when in use with the accessory illustrated in FIG. 74;

FIG. 74D illustrates a cross-section of the collapsible conduit of FIG. 14C in a collapsed configuration;

FIG. 75 is a perspective view of an accessory according to another embodiment of the present disclosure;

FIG. 75a is a perspective view of the embodiment of FIG. 75, when in use with a patient interface;

FIG. 76 is a perspective view of an accessory according to another embodiment of the present disclosure;

FIG. 77 is a perspective view of an accessory according to another embodiment of the present disclosure and when the accessory is in an open position;

FIG. 78 is a perspective view of an accessory according to another embodiment of the present disclosure and when the accessory is in a closed position;

FIGS. 79 and 80 are side-sectional views of an uncollapsed configuration and a collapsed configuration respectively, of another embodiment accessory according to the present disclosure when in use with a patient interface;

FIG. 81 is a perspective view of an accessory according to another embodiment of the present disclosure comprising a backing plate and a pivot member;

FIG. 82 is a perspective view of an alternative pivot member for use with the backing plate shown in FIG. 81;

FIGS. 83 and 84 are side-sectional views of an uncollapsed configuration and a collapsed configuration respectively, of the embodiment of FIG. 21 when in use with a patient interface;

FIG. 84A is a perspective view of an accessory according to another embodiment of the present disclosure;

FIG. 84B is a perspective view of an accessory according to another embodiment of the present disclosure;

FIG. 84C is a perspective view of an accessory according to another embodiment of the present disclosure;

FIG. 84D is a perspective view of an accessory according to another embodiment of the present disclosure;

FIG. 85 is a perspective view of an accessory according to another embodiment of the present disclosure;

FIG. 86 is a perspective view of the embodiment of FIG. 85, when fitted to a patient interface;

FIG. 86a is a side view of the arrangement shown in FIG. 86;

FIG. 87 is a perspective view of an accessory according to another embodiment of the present disclosure;

FIGS. 88 and 89 are side-sectional views of an uncollapsed configuration and a collapsed configuration respectively, of the embodiment of FIG. 87, when in use with a patient interface;

FIG. 90 is a perspective view of an accessory according to another embodiment of the present disclosure;

FIG. 91 is a perspective view of an accessory according to another embodiment of the present disclosure;

FIG. 92 is a perspective view of an accessory according to another embodiment of the present disclosure;

FIGS. 93 and 94 are side-sectional views of an uncollapsed configuration and a collapsed configuration respectively, of the embodiment of FIG. 92 in use with a patient interface;

FIG. 95 is a perspective view of an accessory according to another embodiment of the present disclosure;

FIG. 96 is a perspective view of the embodiment of FIG. 95 accessory in cooperative use with the embodiment shown in FIG. 66 and in use with a patient interface;

FIG. 97 is a perspective view of an accessory according to another embodiment of the present disclosure;

FIG. 98 is a perspective view of an accessory according to another embodiment of the present disclosure;

FIG. 99 is a perspective view of an accessory according to another embodiment of the present disclosure;

FIG. 100 is a perspective view of the embodiment of FIG. 99, when fitted to a patient interface;

FIG. 101 is a side view of the embodiment of FIG. 99, when in use with a patient interface during application of a patient mask onto the patient interface;

FIG. 101a is a closer view of a section of FIG. 101 showing a cuff of the patient mask and a collapsible portion of the patient interface, immediately prior to contact between the collapsible portion and the mask cuff;

FIG. 101b shows the arrangement of FIG. 101a when a pulling force is applied to the accessory and a pushing force is applied to the collapsible portion via the mask cuff;

FIG. 102 is a side view of an accessory comprising a gas path connector, according to another embodiment of the present disclosure;

FIG. 103 is a perspective view of an accessory according to another embodiment of the present disclosure;

FIG. 104 is a perspective view of the embodiment of FIG. 103, when fitted onto a patient interface;

FIG. 105 is a side view of the embodiment of FIG. 103, when in use with the patient interface;

FIG. 106 is a perspective view of an accessory according to another embodiment of the present disclosure; and

FIGS. 107 and 108 are perspective views of the embodiment of FIG. 106, when fitted to a patient interface.

### DETAILED DESCRIPTION

Various embodiments are described with reference to the Figures.

Throughout the Figures and specification, the same reference numerals may be used to designate the same or similar components, and redundant descriptions thereof may be omitted.

In this specification, “high flow”, “high flows”, “high-flow” or other equivalent terminology means, without limitation, any gas flow with a flow rate that is higher than usual/normal, such as higher than the normal inspiration flow rate of a healthy patient. Alternatively, or additionally, it can be higher than some other threshold flow rate that is relevant to the context—for example, where providing a gas flow to a patient at a flow rate to meet or exceed inspiratory demand, that flow rate might be deemed “high flow” as it is higher than a nominal flow rate that might have otherwise been provided.

“High flow” is therefore context dependent, and what constitutes “high flow” depends on many factors as the health state of the patient, type of procedure/therapy/support being provided, the nature of the patient (big, small, adult, child) and the like. Those skilled in the art know from context what constitutes “high flow”. It is a magnitude of flow rate that is over and above a flow rate that might otherwise be provided.

But, without limitation, some indicative values of high flow can be as follows.

In some configurations, delivery of gases to a patient at a flow rate of greater than or equal to about 5 or 10 litres per minute (5 or 10 LPM or L/min).

In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source or flow modulator, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy and/or respiratory support. Gases delivered may comprise a percentage of oxygen. In some configurations, the percentage of oxygen in the gases delivered may be about 15% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “high flow” for premature/infants/paediatrics (with body mass in the range of about 1 to about 30 kg) can be different. The flow rate can be set to 0.4-8 L/min/kg with a minimum of about 0.5 L/min and a maximum of about 70 L/min. For patients under 2 kg maximum flow may be set to 8 L/min.

High flow has been found effective in meeting or exceeding the patient's normal real inspiratory flow, to increase oxygenation of the patient and/or reduce the work of breathing. Additionally, high flow therapy and/or respiratory support may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

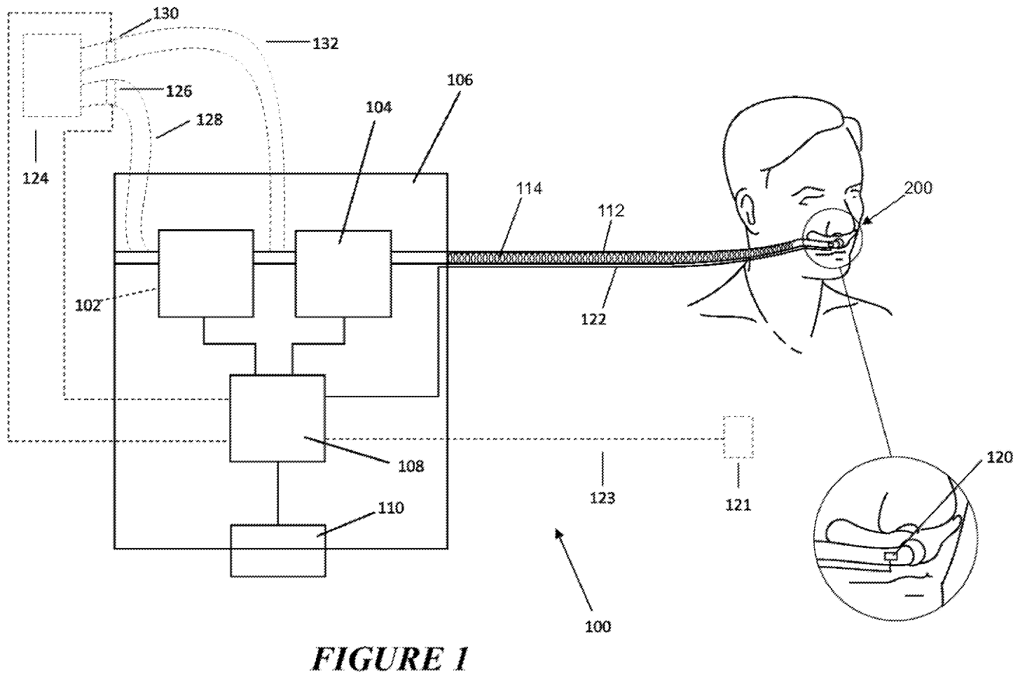

By example, a high flow respiratory system 100 is described below with reference to FIG. 1. High flow may be used as a means to promote gas exchange and/or respiratory support through the delivery of oxygen and/or other gases, and through the removal of CO₂ from the patient's airways. High flow may be particularly useful prior to, during or after a medical and/or anaesthetic procedure.

When used prior to a medical procedure, high gas flow can pre-load the patient with oxygen (i.e. increase the reservoir of oxygen in the blood) so that their blood oxygen saturation level and volume of oxygen in the lungs is higher than normal in order to provide an oxygen buffer while the patient is in an apnoeic phase during the medical procedure.

A continuous supply of oxygen is important to sustain healthy respiratory function during medical procedures (such as during anaesthesia) where respiratory function might be compromised (e.g. diminishes or stops). When this supply is compromised, conditions such as hypoxia and/or hypercapnia can occur. During medical procedures such as anaesthesia and/or sedation, patient breathing is monitored to detect if spontaneous breathing is diminished or ceases. If oxygen supply and/or CO₂ removal is compromised, the clinician stops the medical procedure and facilitates oxygen supply and/or CO₂ removal. This can be achieved for example by manually ventilating the patient for example through bag mask ventilation, or by providing a high flow of gases to the patient's airway using a high flow respiratory system. Further, it will be appreciated that a mask that is used for sedation/ventilation (not necessarily limited to a bag mask) may also be used for pre-oxygenation and also for monitoring patient parameters such as end tidal CO₂, etc.

Further advantages of high gas flow can include that the high gas flow increases pressure in the airways of the patient, thereby providing pressure support that opens airways, the trachea, lungs/alveolar and bronchioles. The opening of these structures enhances oxygenation, and to some extent assists in removal of CO₂ and/or can help support patients with collapsed areas of the lung.

When humidified, the high gas flow can also prevent airways from drying out, mitigating mucociliary damage, reducing risk of infection and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

FIG. 1 shows a respiratory support system 100. The system 100 may be configured to provide high flow respiratory support and/or high flow therapy. The respiratory support system 100 comprises a flow generator 102. The flow generator 102 is configured to generate gas flows that are passed through the respiratory support system 100. The flow generator 102 passes the air to a humidifier 104. The humidifier 104 is configured to heat and humidify gas flows generated by the flow generator 102. In some configurations, the flow generator 102 comprises a blower adapted to receive gases from the environment outside of the respiratory support system 100 and propel them through the respiratory support system 100. In some configurations, the flow generator 102 may comprise some other gas generation means. For example, in some configurations, the flow generator 102 may comprise a source available from a hospital gas outlet (e.g. oxygen or air), or one or more containers of compressed air and/or another gas and one or more valve arrangements adapted to control the rate at which gases leave the one or more containers. As another example, in some configurations, the flow generator 102 may comprise an oxygen concentrator. In some configurations, the flow generator 102 may be adapted to deliver a high flow respiratory support and/or high flow therapy. In some embodiments, the flow source may include a compressed gas source, a device that modifies the flow from a compressed gas source and/or a flow generator which generates a gas flow.

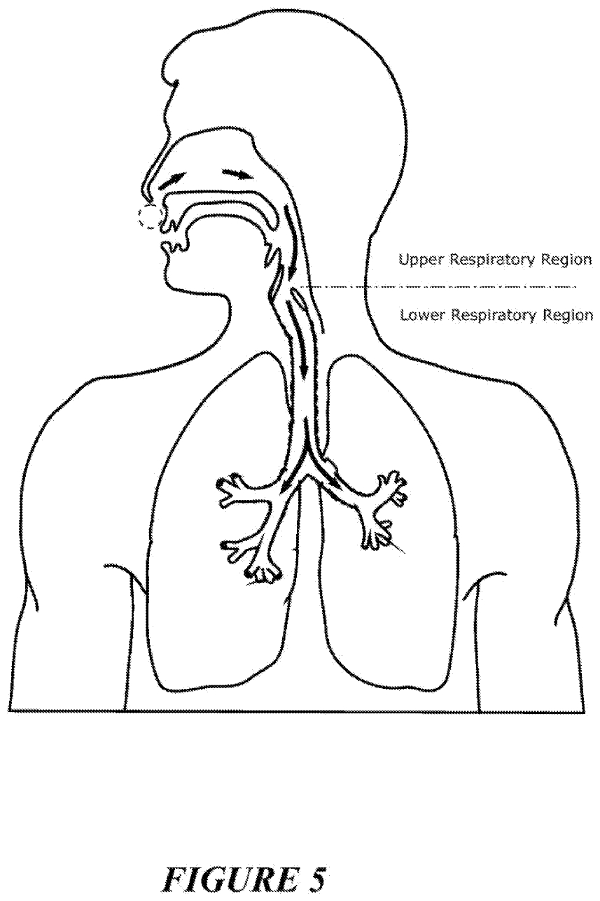

FIG. 5 shows a typical airway of a person, and includes arrows to indicate how a relatively high flow rate of gases supplied to a user may be utilised to effectively push or drive the supplied gases further or deeper into a user's airway than when the person is under normal or typical self-driven respiratory conditions, or when a patient has a diminished respiratory drive.

The respiratory support system 100 comprises a housing 106 that at least partially houses both the flow generator 102 and the humidifier 104 (e.g. the respiratory support system 100 may comprise an integrated flow generator/humidifier apparatus). In other configurations the flow generator 102 and humidifier 104 may have separate housings. A hardware controller 108 is shown to be in electronic communication with the flow generator 102 and the humidifier 104, although in some configurations the hardware controller 108 might only communicate with the flow generator 102 or the humidifier 104. The hardware controller 108 may comprise a microcontroller or some other architecture configured to direct the operation of controllable components of the respiratory support system 100, including but not limited to the flow generator 102 and/or the humidifier 104.

An input/output module 110 is shown to be in electronic communication with the controller 108. The input/output module 110 may be configured to allow a user to interface with the controller 108 to facilitate the control of controllable components of the respiratory support system 100, including but not limited to the flow generator 102 and/or the humidifier 104, and/or view data regarding the operation of the respiratory support system 100 and/or its components. The input/output module 110 might comprise, for example, one or more buttons, knobs, dials, switches, levers, touch screens, speakers, displays and/or other input or output peripherals that a user might use to view data and/or input commands to control components of the respiratory support system 100.

As further shown in FIG. 1, a supplementary gas source 124 may be used to add one or more supplementary gases to the gases flowing through the respiratory support system 100. The one or more supplementary gases join the gas flow generated by the flow generator 102. The supplementary gas source 124 may be configured to deliver one or more supplementary gases including but not limited to air, oxygen (O₂), carbon dioxide (CO₂), nitrogen (N₂), nitrous oxide (NO), anaesthetic agents and/or heliox (a mixture of helium and oxygen). The supplementary gas source 124 may deliver the one or more supplementary gases via a first supplementary gas conduit 128 to or towards the flow generator 102, and/or may deliver the one or more supplementary gases via a second supplementary gas conduit 132 to a location in the flow passage between the flow generator 102 and the humidifier 104. One or more supplementary flow valves 126, 130 may be used to control the rates at which the one or more supplementary gases can flow from the supplementary gas source 124 and through the first and/or second supplementary gas conduits 128, 132. One or more of the supplementary flow valves 126, 130 may be in electronic communication with the controller 108, which may in turn control the operation and/or state of the one or more supplementary flow valves 126, 130. In other configurations, the supplementary gas source 124 may be configured to add one or more supplementary gases downstream of the humidifier 104.

As shown in FIG. 1, a conduit 112 extending from the humidifier 104 links the humidifier 104 to a patient interface 200. The conduit 112 may comprise a conduit heater 114 adapted to heat gases passing through the conduit 112. In other configurations the conduit heater 114 may not be present. In some embodiments, an optional filter (not shown) is arranged between conduit 112 and patient interface 200. The patient interface 200 is shown to be a nasal cannula, although it should be understood that in some configurations, other patient interfaces may be suitable. For example, in some configurations, the patient interface 200 may comprise a sealing or non-sealing interface, and may comprise a nasal mask, an oral mask, an oro-nasal mask, a full face mask, a nasal pillows mask, a nasal cannula, an endotracheal tube, tracheostomy tube, a combination of the above or some other gas conveying system. In an embodiment, the patient interface 200 is a non-sealing interface such as a nasal cannula, which allows gases to be exchanged with the environment. For example, the non-sealing cannula allows carbon dioxide to be removed and/or cleared from the patient's airways while the patient receives a gas flow from the system 100. Further, in some embodiments, the patient interface 200 is in the form of a nasal interface, such that the system does not interfere with other oral airway equipment and/or devices, for example, a tracheal tube in an intubation procedure.

Accordingly, the patient may continue to receive gas flow throughout the intubation procedure. In other embodiments, the patient interface 200 is an oral interface, for example an oral interface that is received in a user's mouth. An oral interface may be preferred in situations involving medical procedures via the nose, such that the interface does not interfere with nasal airway equipment and/or devices, for example a tracheal tube used in a nasal intubation procedure. In other embodiments the interface may be suitable for both nasal and oral placement or may be adapted between a nasal and an oral configuration.

As shown, in some configurations the patient interface 200 may also comprise a gas sensing module 120 adapted to measure a characteristic of gases passing through the patient interface 200. The gas sensing module 120 could be located elsewhere within the gas delivery system and, for example, at the breathing conduit or humidifier. In some embodiments, there may be one or more gas sensing modules 120. In other configurations the gas sensing module 120 could be positioned and adapted to measure the characteristics of gases at or near other parts of the respiratory support system 100. The gas sensing module 120 may comprise one or more sensors adapted to measure various characteristics of gases, including but not limited to pressure, flow rate, temperature, absolute humidity, relative humidity, enthalpy, gas composition, oxygen concentration, carbon dioxide concentration (e.g. for determining end tidal CO₂), and/or nitrogen concentration. Gas properties determined by the gas sensing module 120 may be utilized in a number of ways, including but not limited to closed loop control of parameters of the gases. For example, in some configurations flow rate data taken by a gas sensing module 120 may be used to determine the instantaneous flow, which in turn may be used to determine the respiratory cycle of the patient to facilitate the delivery of flow in synchronicity with portions of the respiratory cycle. The gas sensing module 120 may communicate with the controller 108 over a first transmission line 122. In some configurations, the first transmission line 122 may comprise a data communication connection adapted to transmit a data signal. The data communication connection could comprise a wired data communication connection such as but not limited to a data cable, or a wireless data communication connection such as but not limited to Wi-Fi or Bluetooth. In some configurations, both power and data may be communicated over the same first transmission line 122. For example, the gas sensing module 120 may comprise a modulator that may allow a data signal to be ‘overlaid’ on top of a power signal. The data signal may be superimposed over the power signal and the combined signal may be demodulated before use by the controller 108. In other configurations the first transmission line 122 may comprise a pneumatic communication connection adapted to transmit a gas flow for analysis at a portion of the respiratory support system 100.

Additionally as shown a physiological sensor module 121 may be present. The physiological sensor module 121 may be configured to detect various characteristics of the patient or of the health of the patient, including but not limited to heart rate, EEG signal, EKG/ECG signal, inertial sensors attached to the patient (e.g. to the chest) to detect movement, blood oxygen concentration (via, for example, a pulse oximeter), blood CO2 concentration, transcutaneous CO2 (TcC02) and/or blood glucose. Similarly, the physiological sensor module 121 may communicate with the controller 108 over a second transmission line 123. The second transmission line 123 may comprise wired or wireless data communication connections similarly to the first transmission line 122, and power and data may be communicated similarly. The physiological sensor module 121 may be used, for example, to determine the blood oxygen saturation of the patient.

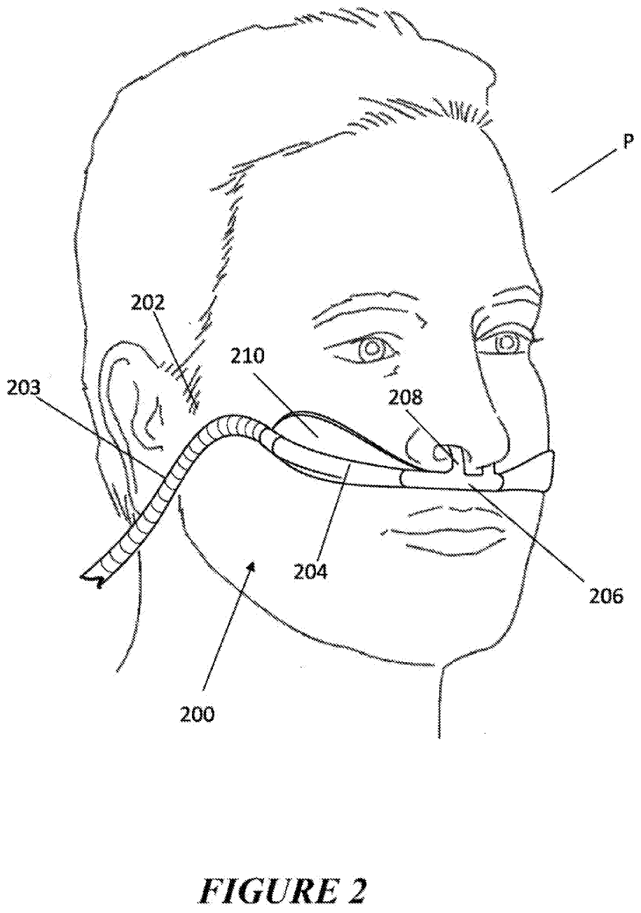

FIG. 2 shows a user or patient P wearing a patient interface 200, for example the patient interface 200 of the respiratory system of FIG. 1. The patient depicted is an adult, however, the patient may be an infant or juvenile. In the illustrated non-limiting configuration, the patient interface 200 is a nasal cannula. The patient interface 200 comprises a first gas conduit 202. The first gas conduit 202 is adapted to receive gases from the respiratory support system 100 (for example, via the conduit 112 shown in FIG. 1) and channel the gases to the patient P. The first gas conduit 202 may comprise a reinforcement element 203 adapted to strengthen and/or add rigidity to the first gas conduit to prevent deformation or collapse of the first gas conduit 202 arising due to the application of forces against the first gas conduit 202. The reinforcement element 203 may include a number of structures, including but not limited to plastic or metallic reinforcing beads that lie in or on the wall of the first conduit lumen 202.

The first gas conduit 202 is in pneumatic communication with a flow manifold 206. The flow manifold 206 receives gases from the first gas conduit 202 and passes them to one or more nasal delivery elements 208 (e.g. nasal prongs). The one or more nasal delivery elements 208 extend outwardly from the flow manifold 206. The one or more nasal delivery elements 208 are adapted to be non-sealing when positioned in one or more nares of the patient P. As shown, the patient interface 200 comprises two nasal prongs 208 adapted to be positioned one in each of the patient's nares. Each nasal prong 208 may be shaped or angled such that it extends inwardly towards a septum of the patient's nose. Alternatively the first patient interface 200 may be a sealing nasal interface.

In the embodiment shown in FIG. 2, the flow manifold 206 receives flow from one lateral side of the flow manifold 206 (e.g. with respect to an imaginary vertical plane bisecting the face of the patient P) and channels flow to the manifold and each of the nasal prongs 208. In an example, the flow manifold 206 receives flow from a single side of the flow manifold 206 and channels flow to the manifold and each of the nasal prongs 208. In some embodiments a conduit may extend from the left hand side or from the right hand side of the manifold. In some situations providing the conduit on the left hand side of the patient interface may be preferred for access for a clinician, for example for intubation. Alternatively, a conduit extending from the right hand side may be preferred, for example in procedures such as endoscopies where the patient is typically lying on his or her left hand side. In other configurations, the patient interface 200 may comprise greater (for example, three or four) or fewer (for example, one) nasal delivery elements 208. In other configurations, each nasal delivery elements 208 can have different properties. For example, one of a pair of nasal delivery elements 208 can be relatively long and the other nasal delivery elements 208 can be relatively short.

In some configurations, the flow manifold 206 may be configured to receive flow from two lateral sides of the flow manifold 206 (e.g. from a ‘left’ and ‘right’ of the flow manifold 206 instead of just the patient's right hand side of the flow manifold 206 as seen in FIG. 2). In some such configurations, multiple gas conduits may be used to provide for pneumatic communication between the flow manifold 206 and the respiratory support system 100. For example, the patient interface may comprise dual conduits, the first gas conduit 203 extending from a first side of the interface (in the illustrated example the right hand side of the patient) and a second gas conduit extending from a second opposite side of the interface. In some configurations, the flow manifold 206 may be configured to receive flow from a non-lateral side of the flow manifold 206 (e.g. from a ‘bottom’ or ‘top’ of the flow manifold 206).

The patient interface may further comprise mounts and/or supports, e.g., cheek supports 210, for attaching and/or supporting the gas conduit 202 or conduits on the patient's face. Alternatively or additionally, the patient interface may be held in place via one or more headstraps or headgear.

The first gas conduit 202 of the patient interface 200 comprises a first portion 204 configured to transition from a first configuration in which a first level of gases is able to pass through the first portion 204 to a second configuration in which a second level of gases is able to pass through the first portion 204.

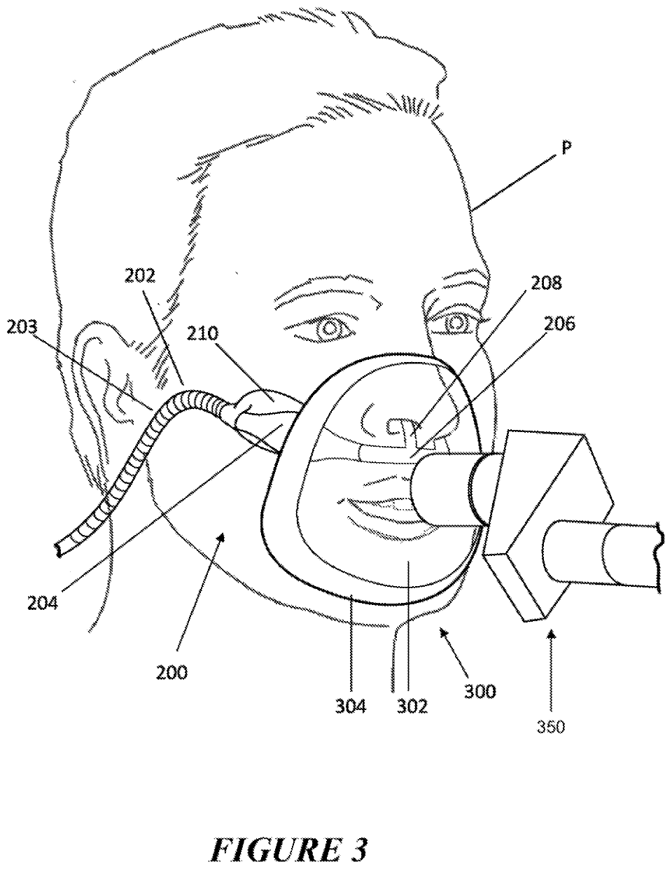

FIG. 3 shows a non-limiting exemplary embodiment of a patient P wearing the patient interface 200 as shown in FIG. 2 (a first patient interface) underneath a face mask 300 assembly (a second patient interface). FIG. 3 schematically shows the face mask as a transparent structure in order to illustrate the patient interface 200 under it. The first patient interface 200 may be used with a first respiratory support subsystem and the second patient interface 300 may be used together with a second respiratory support subsystem. In some embodiments, the first patient interface 200 and second patient interface 300 may be used with the same respiratory support system.

A system may find benefit in the selective delivery of separate respiratory supports and/or therapies to a patient using different patient interfaces, and/or in stopping or ceasing the delivery of a respiratory support and/or therapy from an interface and/or allowing gases provided by an interface to be sampled.

The system and devices as described find particular application in emergency resuscitation, around intubation of a patient receiving high flow respiratory support and/or therapy, ear, nose, and throat (ENT) surgery, in assisting with conditioning of a patient in a pre-operative state prior to administration of anaesthetics, and during post-extubation and recovery.

Face mask assembly 300 may be used as or with a second respiratory support subsystem and/or to deliver one or more substances other than a substance delivered by the cannula 200, for example anaesthetic agents or oxygen, to the patient, or the same substance but at different flow and/or pressure levels. Alternatively, the face mask assembly 300 may be used to stop the delivery of respiratory support and/or therapy from a first respiratory support subsystem. The face mask assembly 300 may also be adapted to measure respiratory gases, for example exhaled carbon dioxide from the patient, the measurements of which may otherwise be affected by flow from the patient interface 200 of the first respiratory support subsystem.

Accordingly, the embodiment shown in FIG. 3 allows for the alternation between the two different respiratory support subsystems. Additionally, this configuration may allow the patient interface 200 to be left on the patient throughout the surgical procedure and/or into recovery (whether or not the patient continues to receive a gas flow through the patient interface 200 throughout the procedure) without interfering with other clinical practices.

In the embodiment shown, face mask assembly 300 comprises a full face mask 302 configured to cover both the patient's nose and mouth. In other configurations, the face mask 300 may be a nasal mask which is placed over the patient interface 200 to cover only the patient's nasal region.

As shown, the face mask 302 comprises a seal region 304 adapted to seal against the patient's face. The face mask assembly 300 is connected to a second gas source, for example via a filter element 350 or a humidity moisture exchanger (not shown), which supplies the one or more other gases to the patient via the face mask. That is, the second gas source is preferably different from the source supplying gas (for example, supplementary gas source 124/flow generator 102) to the patient interface 200. In other embodiments, the patient interface 200 and the face mask assembly 300 are connected to a common gas source.

In an embodiment, the face mask assembly 300 is connected to a separate gas source or a separate respiratory support device. For example, the respiratory support can be a ventilator or a CPAP or a high flow respiratory support and/or therapy device or a manual resuscitator (for example a hand-held face mask with bag). Alternatively or in addition, the face mask assembly 300 may be connected to a device for measuring a characteristic of respiratory gases.

Alternatively, the mask assembly 300 could be connected to an anaesthetic device and anaesthetic gas, or air, or oxygen, or a combination of gases, can be delivered via the mask 302.

The embodiment shown in FIG. 3 allows for the delivery of gas from multiple sources via at least two different respiratory support modes, and further allows a doctor, clinician or medical professional to quickly and easily change the type of respiratory support mode.

In one particular application, a patient preparing for anaesthesia can be pre-oxygenated by delivering a high flow of oxygen or humidified gases or mixture of both via a nasal cannula. In some circumstances, anaesthesiologists managing the sedation and/or anaesthesia of a patient may want to switch between delivery of gas flow from one patient interface (for example a nasal cannula 200) and delivery of gas flow from another patient interface, such as via a face mask 300.

Anaesthesiologists also use a mask with a bag to oxygenate a patient, and in some instances find it more beneficial to use a bag mask if a patient's vital signs begin to drop for example to deliver more pressure or have greater control over the variation in delivered pressure. In some situations a medical professional may wish to switch between different respiratory systems or support modes. In a first mode respiratory support may be provided by a first respiratory support system (for example via the patient interface 200) and in a second mode respiratory support may be provided by a second respiratory support system (for example via the patient interface 300), with the support from the first system reduced or stopped. For example, the additional flow from a high flow provided by nasal interface 200 may also modify the expected behaviour of the anaesthetic circuit provided by the face mask 300, and therefore it may be advantageous to be able to reduce or stop the additional flow from the first respiratory system.

In some configurations, the switching between two respiratory support modes or subsystems may be facilitated by a structure of the first gas conduit 202, which has first portion 204 configured to transition from a first configuration in which a first level of gases is able to pass through the first portion 204 to a second configuration in which a second level of gases is able to pass through the first portion 204.

In some configurations, the first portion 204 is configured to be more collapsible or otherwise better adapted at changing the flow of gas through the first portion 204 (therefore reducing the flow of gas through the conduit and to the patient) than other portions of the conduit 202, and/or allowing a seal of a mask to seal over the top of the conduit. In other configurations the entire conduit may be configured to be collapsible. In some configurations a vent arrangement may be provided to vent gases from the conduit to atmosphere.

In some embodiments, the first configuration or first condition is a substantially open configuration and the second configuration or second condition is a substantially closed configuration. That is, the conduit 202 is configured to be more collapsible, deformable or otherwise adapted to fully close off the flow at the first portion 204 than at other portions of the conduit 202. In the second condition, gases to the nasal delivery elements 208 may be reduced or stopped.

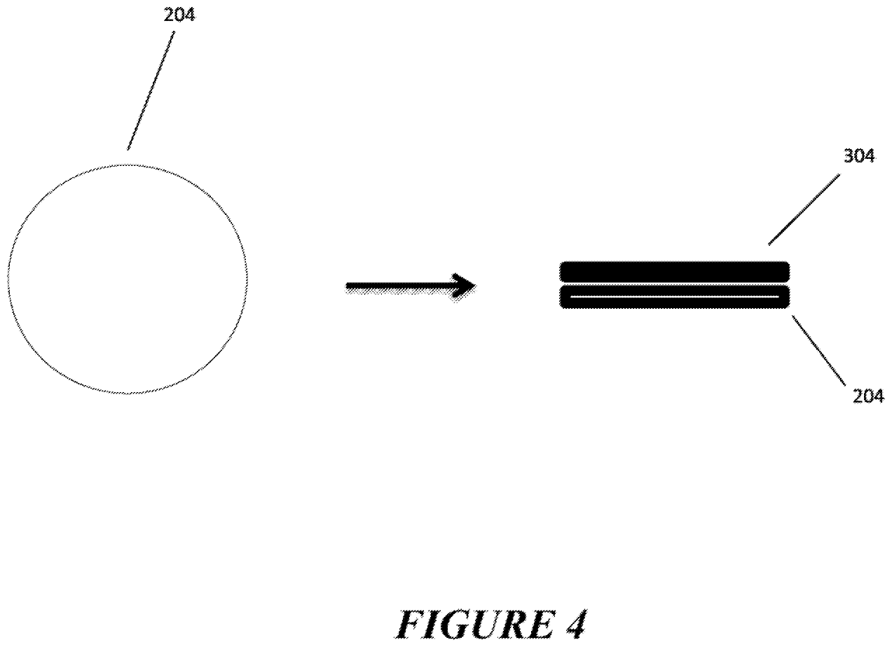

FIG. 4 shows one example of this configuration, in which the conduit (for example the conduit 204 of the nasal cannula 200 of FIG. 3) at a first portion 204 is substantially closed by the seal 304 of face mask 302. In such an embodiment, the first portion (i.e. the more collapsible or deformable section) of the first gas conduit should be of a length that is greater or equal to a width of a section of a seal of the face mask that bears over the first portion of the first gas conduit. This may provide that the seal of the face mask does not bear over a non-collapsible section of the first gas conduit. For example, the first portion may extend from a distance of 35 mm or less from the centre of a user's nose to at least 50 mm from the centre of a user's nose, The first portion 204 may have a length of at least about 5 mm, about 1 mm to about 30 mm in length, or about 5 mm to about 15 mm in length, or about 10 mm in length. In some embodiments the length of the first portion may be at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 20 mm, 25 mm, 30 mm, 35 mm, 40 mm, 45 mm, 50 mm or greater.

The first portion 204 may progress between the first and second configurations based on a relative level of force applied to a wall of the first portion 204. For example, as shown in FIG. 3, the force may be applied by the seal 304 of face mask 302. In this example, first portion 204 is configured to be positioned under the seal 304 of the face mask 302.

Alternatively, the force may be applied to first portion 204 by other means, e.g., clamps (not shown), or alternatively a medical practitioner may compress the conduit by pressing on the conduit wall with a finger or thumb.

In some embodiments, the seal of the face mask acting on the first portion of the gas conduit causes the first portion to form a seal or at least a partial seal between the nasal outlets of the first patient interface 200 and the flow generator 102. Additionally, the seal of the face mask forms a seal or at least a partial seal over the first portion of the gas conduit.

Switching between respiratory support therapies is therefore achieved simply by applying a mask to the patient's face so that the seal of the mask collapses (partially or completely) the first portion of the gas conduit of the first interface 200 to ‘stop’ or ‘turn off’ or reduce the respiratory support and/or therapy supplied by the first interface 200 and also provides a seal between the face mask 300 and the external surface of the first portion 204 of the conduit 202 such that respiratory support and/or therapy can be provided by the mask 300 with the respiratory support and/or therapy provided by the first interface is stopped or reduced. As noted, the first portion 204 of the patient interface 200 is configured to be collapsible and will hereinafter be referred to as collapsible portion 204.

The cannula with a collapsible conduit portion allows a user, e.g. an anaesthetist or a nurse or a clinician to use a mask and prevent delivery of gases from multiple sources (e.g. the mask and cannula). The first interface 200 is structured and functions in a manner to reduce or close the delivery of high flow and other respiratory support and/or respiratory therapy or anaesthesia gases through a mask when the interface 200 is moved to a collapsed configuration. In some embodiments the removal of the mask from the patient's face allows the respiratory support and/or therapy supplied by the first interface to recommence, as the conduit returns from the collapsed configuration to the open configuration.

### Patient Interface Gas Sampling

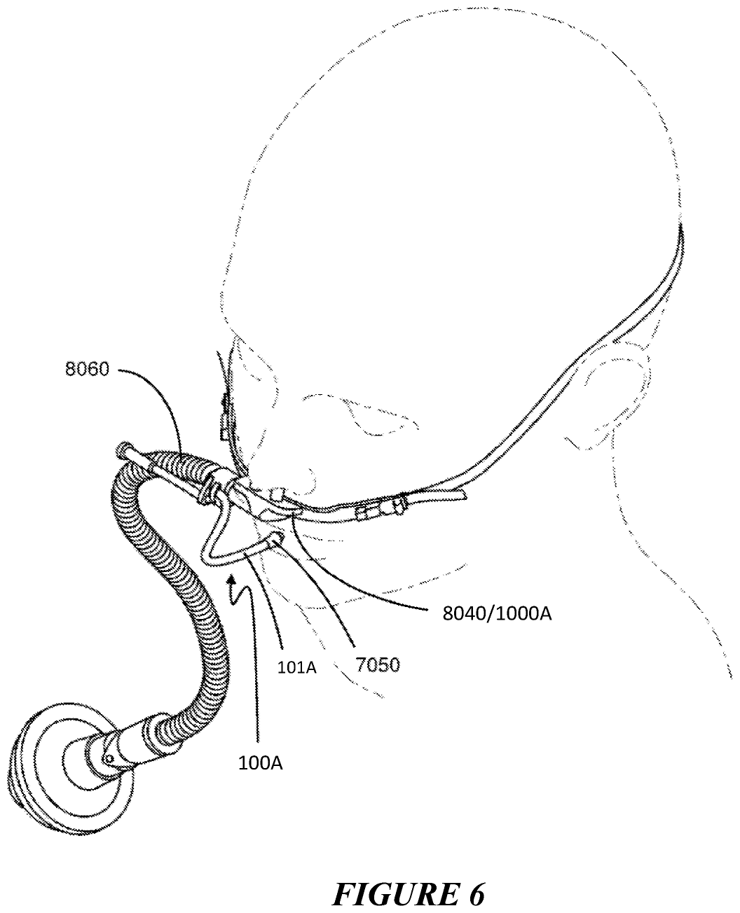

FIG. 6 is a schematic view of a patient wearing a gas delivery system comprising a breathing apparatus 1000A comprising a nasal cannula 8040 configured to deliver gases to a patient that are supplied via a gas delivery tube 8060. A gas sampling interface 100A includes a gas sampling conduit 101A in which the tip 7050 of the gas sampling conduit 101A is positioned proximate to the patient's mouth and/or nose to sample exhaled and/or expired gases from the mouth.

The gas sensing module 120 shown in FIG. 1 is shown to be located on a manifold portion of a patient interface and, in some embodiments, could form part of the gas sampling interface of the present disclosure. For example, the gas sensing module 120 could be utilised to supplement data provided by a respiratory gas monitor in fluid communication with the sampling outlet of the gas sampling interface. The gas sensing module 120 could form part of the gas sampling interface and could, for example, comprise a flow meter in the sampling conduit. The gas sensing module 120 could comprise a capnography sensor in the sampling conduit or at the sampling outlet. The gas sampling interface may therefore be configured for use in the application of mainstream capnography. In this instance, the sampling outlet may vent patient gas flow to ambient once downstream of the sensing module 120 and in this manner the sampling outlet is configured to deliver the patient gas flow away from the patient in order to enable flow through the conduit and past the sensor. The gas sensing module 120 may be connected via a wired or wireless data communication to an appropriate receiver capable of displaying data collected by the sensing module 120 to a clinician.

The gas sensing module 120 could be a separate component to the gas sampling interface and could be located elsewhere on the patient interface. The gas sensing module 120 could comprise a gas composition sensor and could be located in or at the sampling conduit and/or in or at the sampling outlet.

Alternatively, the gas sampling interface may include no sensors at the manifold portion nor any sensors at the patient. For example, the gas sampling interface may be configured for only the sampling inlet to be located at the patient and for the patient gas analysis to occur away from the patient, for example at a respiratory gas monitor. The gas sampling interface may therefore be used in the application of sidestream capnography. In this instance, the sampling outlet may facilitate delivery of the patient gas flow away from the patient and towards a respiratory gas monitor. The omission of sensors at the patient may in some instances improve accessibility for medical instruments.

In alternative embodiment (not illustrated), the gas sampling interface could include a passive sampling configuration and, for example, could be configured to sample the patient gases via colourimetry. In this instance, the sampling conduit could be configured to deliver patient gases to an assay of colorimetric reagents or to another form of colourimeter configured to indicate the presence or concentration of one or more particular gases in the patient gas flow. In some configurations, the gas sampling interface may include the colourimetry means in the sampling conduit or at the sampling outlet.

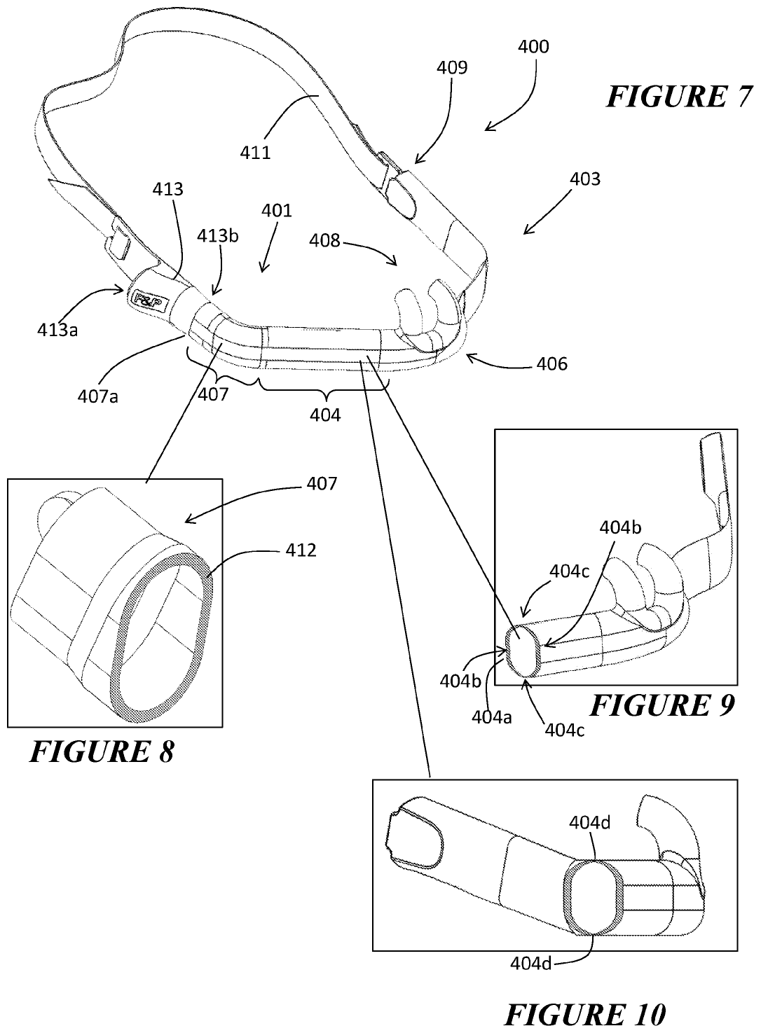

FIGS. 7 to 65 illustrate various embodiments of a collapsible patient interface and/or an accessory for use with a collapsible patient interface which is configured with a gas sampling interface for receiving patient gas flow at the patient.

FIGS. 7 to 10 exemplify a patient interface 400 comprising a nasal cannula and including a gases delivery side member 401 having a hollow interior providing an apparatus gases flow path and configured to deliver apparatus gases to a patient via a manifold 406 to a delivery outlet comprising a pair of nasal prongs 408. The pair of nasal prongs 408 extend from the manifold 406. The gases delivery side member 401 extends from a first side of the manifold 406 and the interface 400 further includes a non-delivery side member 403 extending from a second side of the manifold 406 which is opposite to the first side. The non-delivery side member 403 includes an end 409 configured for connection to a headstrap 411.

The gases delivery side member 401 includes a collapsible portion 404 configured to move from the normally open configuration shown in FIGS. 7, 9 and 10 to a collapsed configuration in which apparatus gas flow through the collapsible portion 404 is reduced or stopped. The collapsible portion 404 is configured to move to the collapsed configuration upon application of a collapsing force such as from a patient mask placed over the patient's face and wherein a seal of the mask is pressed down upon the collapsed portion 404. The gases delivery side member 401 also includes a non-collapsible portion 407 configured to remain open during application of the collapsing force onto the collapsible portion 404. The collapsible portion 404 may comprise or be formed from a thermoplastic elastomer. The gases delivery side member 401 may comprise or be formed from a thermoplastic elastomer.

One end of the non-collapsible portion 407 comprises a delivery inlet 407a for receiving apparatus gas flow. The patient interface 400 further includes a gas path connector 413 which has a rigid structure and includes a delivery inlet 413a and a delivery outlet 413b. The gas path connector delivery inlet 413a is connectable to an apparatus gas supply via a conduit (not shown). The gas path connector delivery outlet 413b is connected to the delivery inlet 407a of the non-collapsible portion 407. The gas path connector 413 is also connected to the headstrap 411 at an opposite end of the headstrap to that which is connected to the headstrap end 409 of the non-delivery side member 403.

FIG. 8 illustrates a cross-section of the non-collapsible portion 407 which includes wall 412 of uniform thickness. FIGS. 9 and 10 illustrate a cross-section of the collapsible portion 404 which includes a wall 404a of non-uniform thickness. The collapsible portion 404 has an elongate cross-section and in particular a stadium-shaped cross section which includes a pair of longitudinal sides 404b extending between a pair of ends 404c. As shown in FIG. 10, a thin-walled portion 404 is provided at each of the ends 404c. The thin wall portions 404d are configured to provide fold lines at which the collapsible portion 404 bends or folds upon application of the collapsing force.

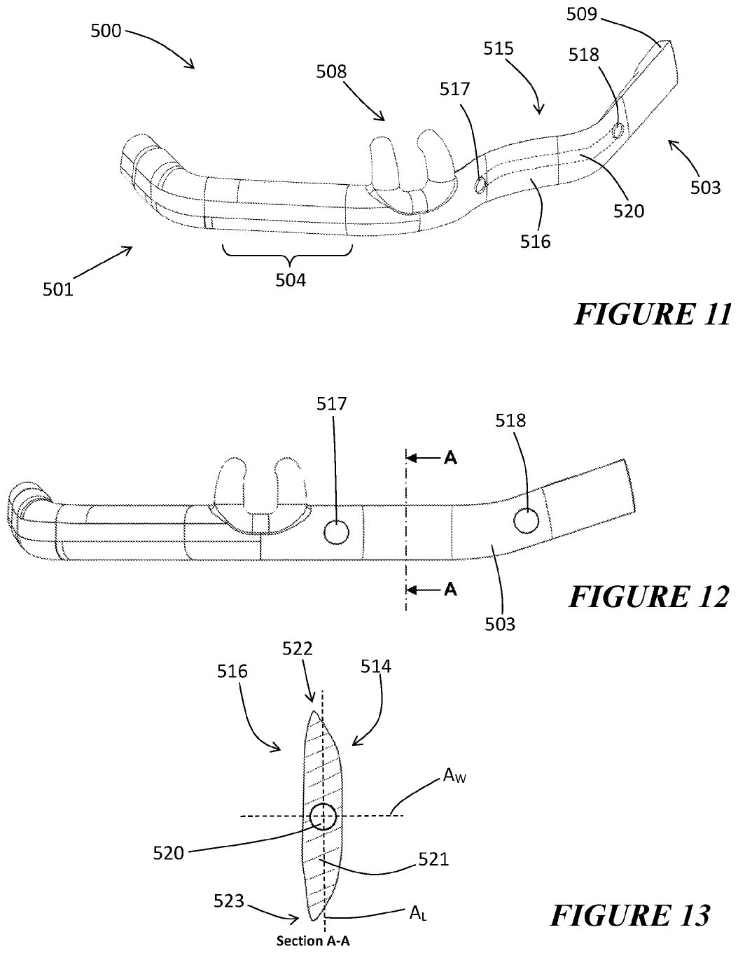

The patient interface 400 illustrate in FIGS. 7-10 provides background reference and context for FIGS. 11 to 68 which illustrate various embodiments of patient interfaces (or parts thereof) similar to interface 400 but which also include a gas sampling interface comprising a sampling inlet for receiving a patient gas flow at the patient, a sampling outlet configured for fluid communication with a respiratory gas monitor and a sampling conduit in fluid communication between the sampling inlet and the sampling outlet. The gas sampling interface facilitates fluid communication between the patient gas flow and a respiratory gas monitor for use in providing patient feedback to a clinician.

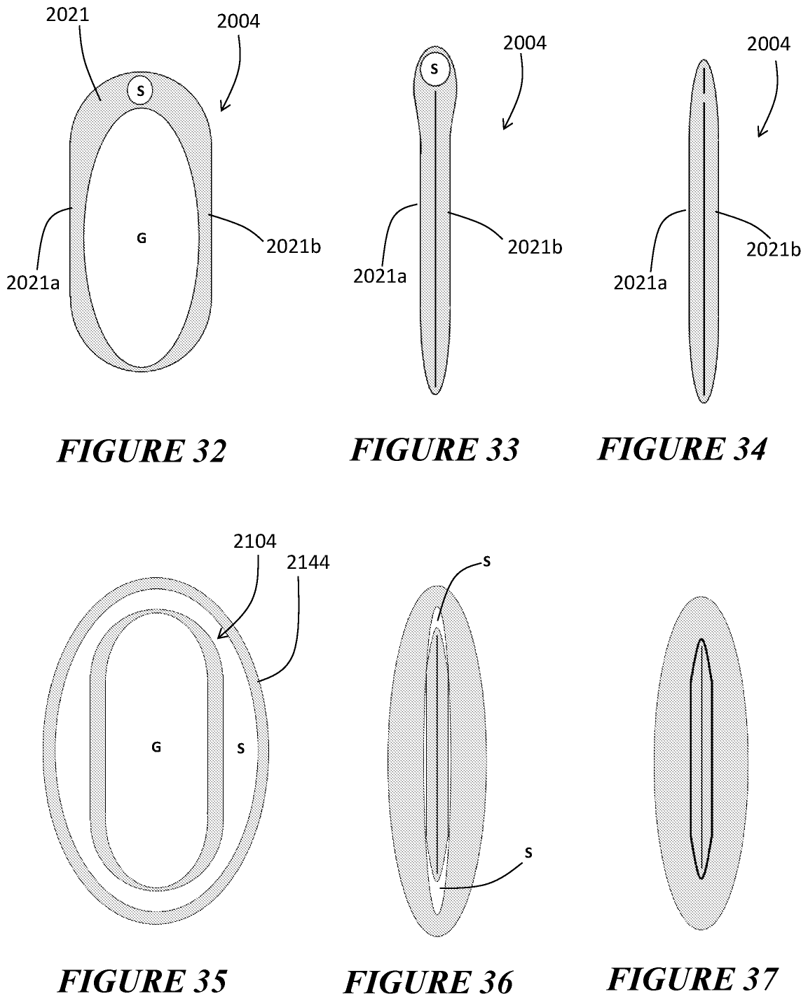

In particular, FIGS. 11 to 31 illustrate embodiments in which a gas sampling interface is provided to (or in lieu of) the non-delivery side member. FIGS. 32 to 60 illustrate embodiments in which a gas sampling interface is provided to the gases delivery side member. The various embodiments of the patient interface each comprise a gases delivery side member which is generally equivalent to gases delivery side member 401 and which each comprise a collapsible portion configured to move between a normally open configuration and a collapsed configuration in which the apparatus gas flow therethrough is reduced or stopped.

FIG. 11 illustrates a perspective view of a patient interface 500 generally equivalent to patient interface 400 but in which the non-delivery side member 503 includes a gas sampling interface 515. The patient interface 500 includes a gases delivery interface comprising a gases delivery side member 501 which includes a collapsible portion. The gases delivery side member 501 is configured to deliver an apparatus gas flow to a patient. The gases delivery interface comprises a delivery outlet comprising nasal prongs 508 to deliver the apparatus gas flow to the patient. The gases delivery interface further comprises a gases delivery side member 501 extending from a first side of the delivery outlet and comprising an apparatus gas flow path in fluid communication with the nasal prongs 508. The collapsible portion 504 configured to move from the normally open configuration shown in FIG. 11 to a collapsed configuration in which apparatus gas flow through the collapsible portion 504 is reduced or closed in order to reduce or stop the apparatus gas flow through the apparatus gas flow path.

The gas sampling interface 515 includes a pair of spaced apart openings formed in a non-patient-facing wall 516 of the non-delivery member 503. The pair of spaced apart openings comprise an inlet port 517 and an outlet port 518 which is in fluid communication with the inlet port 517 via a sampling conduit 520 extending internally through the non-delivery side member 503.

The outlet port is located toward a headstrap end 509 of the non-delivery side member 503 which is configured for coupling to a headstrap. The inlet port 517 is positioned at or near the delivery outlet comprising nasal delivery prongs 508. The sampling conduit 520 extends along a length direction of the non-delivery side member 503. The inlet port 517 provides a sampling inlet through which patient gases can be received at the patient. The outlet port 518 provides a sampling outlet which is configured for fluid connection to a respiratory gas monitor.

In an embodiment, the sampling outlet may be connected in fluid communication with a respiratory gas monitor. The respiratory gas monitor (not shown) may apply suction or pressure or a vacuum through the sampling conduit 520 in order to draw and receive patient gas flow for analysis. In another embodiment, the respiratory gas monitor may passively receive the patient gas flow through the sampling conduit 520 i.e. the respiratory gas monitor may receive patient gas flow without drawing the patient gas flow via suction or pressure or a vacuum etc.

FIG. 12 provides a front view of the patient interface 500 and which better illustrates the spacing between the inlet port 517 and the outlet port 518. FIG. 13 provides a cross-section of the non-delivery side member 503 taken along the section A-A in FIG. 12. The sampling conduit 520 has a substantially circular cross-section and is formed in an otherwise non-hollow solid body 521 of the non-delivery side member 503. The non-delivery side member includes a non-patient-facing wall 516 and a patient-facing wall 514 which, in use, is placed into contact with the patient's face.

The patient-facing wall 514 and the non-patient-facing wall 516 extend between a pair of edges comprising an upper edge 522 and a lower edge 523.

As shown in FIG. 13, the cross-section of the non-delivery side member 503 is elongate and is also asymmetric in at least one axis. The cross-section includes a width axis Aw extending through the patient-facing wall 516 and the non-patient-facing wall 514. The cross-section also includes a length axis AL extending perpendicularly to the width axis Aw. The length axis AL is substantially parallel with the patient-facing wall and also with the non-patient-facing wall.

The cross-section of the non-delivery side member 503 is substantially symmetrical about the width axis Aw. In other embodiments, the cross-section may be asymmetrical and, for example, the sampling conduit 520 could be located closer to one of the upper or lower edges 522, 523. As seen in FIG. 13, the sampling conduit 520 is located substantially centrally in the cross-section such that the sampling conduit 520 is equidistant between the upper and lower edges 522 and 533 and also between the patient-facing wall 514 and the non-patient-facing wall 516.

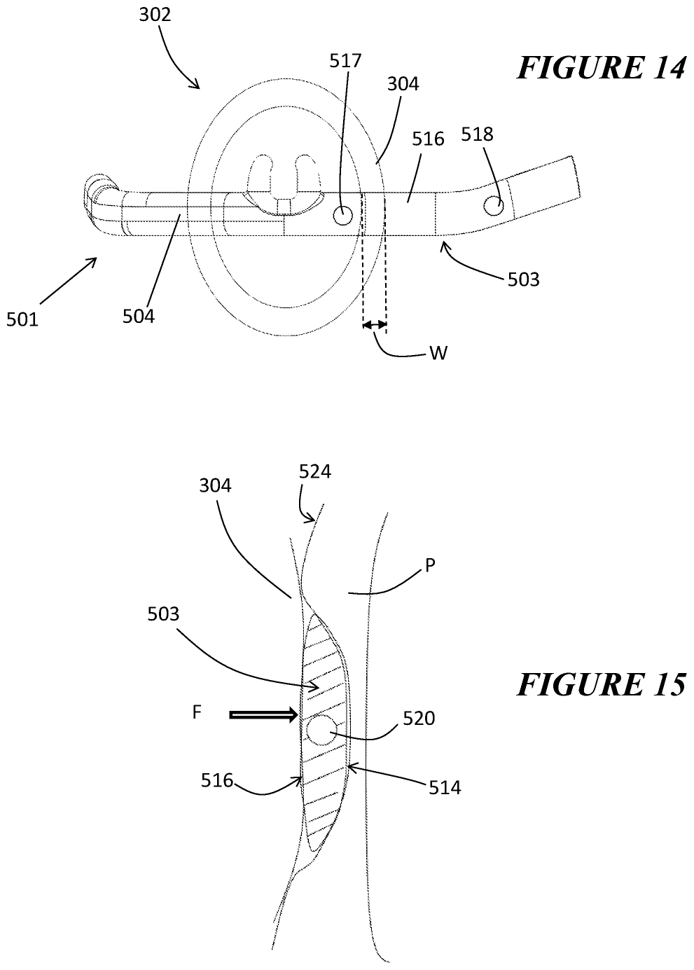

The cross-section of the non-delivery side member 503 is asymmetric about the length axis AL. The asymmetry about the length axis AL is due to the patient-facing-wall 514 having greater curvature as compared to the non-patient-facing wall 516 which is substantially planar. The substantially planar configuration of the non-patient-facing wall 516 may help provide a seal between the non-patient-facing wall 516 and a seal of a patient face mask, which will be discussed in further detail below with reference to FIGS. 14 and 15. In other alternative embodiments, the cross-section of the non-delivery side member 503 could be substantially symmetrical about the length axis AL. The cross-section of the non-delivery side member 503 could be substantially symmetrical about both the length axis AL and the width axis Aw.

Turning to FIG. 14, the inlet port 517 and outlet port 518 are spaced from one another by a distance that is greater than or equal to a width W of a seal 304 of a face mask 302 that is configured for placement on the patient's face and with the seal 304 bearing over a portion of the non-delivery side member 503. In particular, the seal 304 is overlaid onto the non-patient-facing wall 516. Part of the seal 304 of face mask 302 bears over a portion of the gases delivery side member 501 and, in particular, is overlaid onto the collapsible portion 504. Upon application of the face mask 302 (i.e. pressing of the face mask 302 toward the patient's face) the collapsible portion is moved to the collapsed configuration in which the apparatus gas flow through the apparatus gases flow path is reduced or stopped.

The inlet port 517 and outlet port 518 are positioned such that the mask 302 covers only the inlet port 517. In other words, the inlet port 517 lies within the cavity formed by the patient's face and the mask 302 when the mask 302 is applied over the patient interface 500 onto the patient's face. A sampling device (for example a sampling tube comprising the sampling inlet) may connect to the inlet port 517 and fit within the area under the mask 302. Alternatively, the inlet port 517 may not be connected to a sampling device and may itself provide the sampling inlet. The sampling lumen 520 acts as a tunnel under the mask seal 304 so as to provide fluid communication between the inlet port 517 and outlet port 518 and making available at the outlet port 518 a sample of the patient gas flow taken at the inlet port 517 which is located at the patient and also inside of the mask 302.

FIG. 15 illustrates a cross-section of the non-delivery side member 503 sandwiched between the mask seal 304 and the face of the patient P. The mask seal 304 is pressed onto the non-patient-facing wall 516 with a force F (for example applied manually by a clinician or retained by headgear) and causing the non-delivery side member 503 to become partially recessed into the surface 524 of the patient's face and in which the patient-facing wall 514 is recessed below the face surface 524. The substantially planar configuration of the non-patient-facing wall 516 is approximately aligned with the face surface 524 on either side of the non-delivery side member 503. A substantially continuous surface is formed by the non-patient-facing wall 516 and the face surface 524 on either side of the non-delivery side member 503 and thereby facilitating formation of a seal between the mask seal 304, the non-patient-facing wall 516 and the patient's face. In an alternative configuration (not shown) the patient-facing-wall 514 may remain substantially flush with the face surface 524 and the mask seal 304 deforms around the non-delivery side member 503 such that the non-delivery side member 503 does not become recessed into the patient's face to the extent that is illustrated in FIG. 15.

As will be appreciated from FIG. 15, the sampling conduit 520 fits within the cross-section of the non-delivery side member 503 and therefore does not contribute any disruption to the mask seal 304. The sampling conduit 520 is contained within the solid body 521 of the non-delivery side member 503 and is protected from substantial deformation during application of the face mask. The sampling conduit 520 therefore remains open during bearing of the mask seal 304 onto the non-delivery side member 503. In this manner, the sampling conduit 520 is configured to remain open to maintain fluid communication between the inlet port 517 and the outlet port 518 when the collapsible portion 504 is moved to the collapsed configuration. That is, the sampling conduit 520 is configured to remain open when the apparatus gas flow through the collapsible portion 504 is reduced or stopped.

This has usability benefits. For example, this may allow a user to continue monitoring a gas at the patient when a mask 302 is applied over the patient interface 500 without having to disconnect the gas sampling interface 515 from a respiratory gas monitor (e.g. capnography device) and reconnecting the respiratory gas monitor to another sampling interface (for example to a sampling port (not shown) in the mask 302. Additionally or alternatively, this may allow a user to monitor output from a single respiratory gas monitor rather than several monitors connected to various sampling interfaces (for example the gas sampling interface 515 and a sampling port on the mask 302), which could be confusing and thereby increase the risk of incorrect or inaccurate measurements.

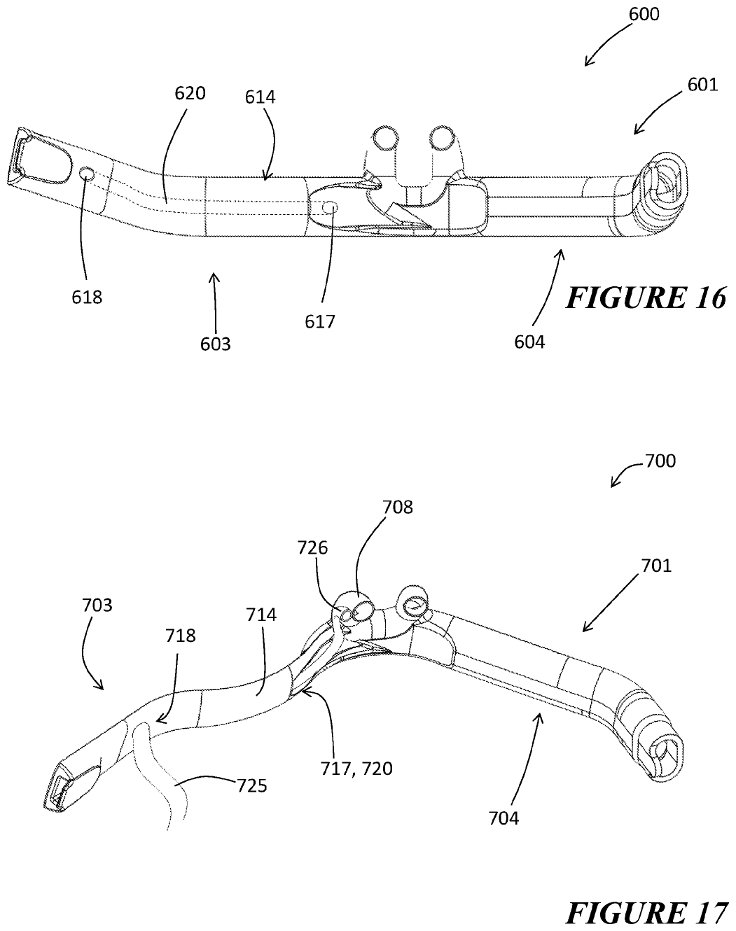

FIG. 16 illustrates an alternative embodiment to patient interface 500 and in which a patient interface 600 comprises a gases delivery side member 601 comprising a collapsible portion 604. The patient interface 600 further comprises an inlet port 617 and an outlet port 618 located in the patient-facing wall 614 of the non-delivery side member 603. In an embodiment, respective sampling tubes could connect to the inlet and outlet ports 617, 618 and for example via a luer lock or threaded connection or plug fit or barb connection. In this embodiment, the sampling conduit is therefore comprised of the passage 620 extending internally through the non-delivery side member between the inlet and outlet ports 617, 618.

FIG. 17 illustrates another alternative embodiment patient interface 700 in which the sampling conduit comprises a sampling line and in particular a sampling tube 725. In an example, the sampling conduit is or forms a part of a sampling line. The tube 725 extends through the internal passage 720 of the non-delivery side member 703 and extends out of each of spaced apart openings in the patient-facing wall 714 which comprise a sampling tube inlet 717 and a sampling tube outlet 718. The portion of the tube 725 which extends from the sampling tube inlet 717 includes a sampling nasal prong 726 formed at the end of the tube 725 and which provides the sampling inlet configured to receive a patient gas flow at the patient. The sampling nasal prong 726 is located adjacent to and extends alongside one of the nasal delivery prongs 708 which is in fluid communication with the gases delivery side member 701 which includes collapsible portion 704. The portion of the tube 725 extending from the sampling tube outlet 718 may comprise the sampling outlet and may be configured for fluid communication with a respiratory gas monitor so as to provide fluid communication. The tube 725 may therefore provide fluid communication between the sampling nasal prong 726 and a respiratory gas monitor.

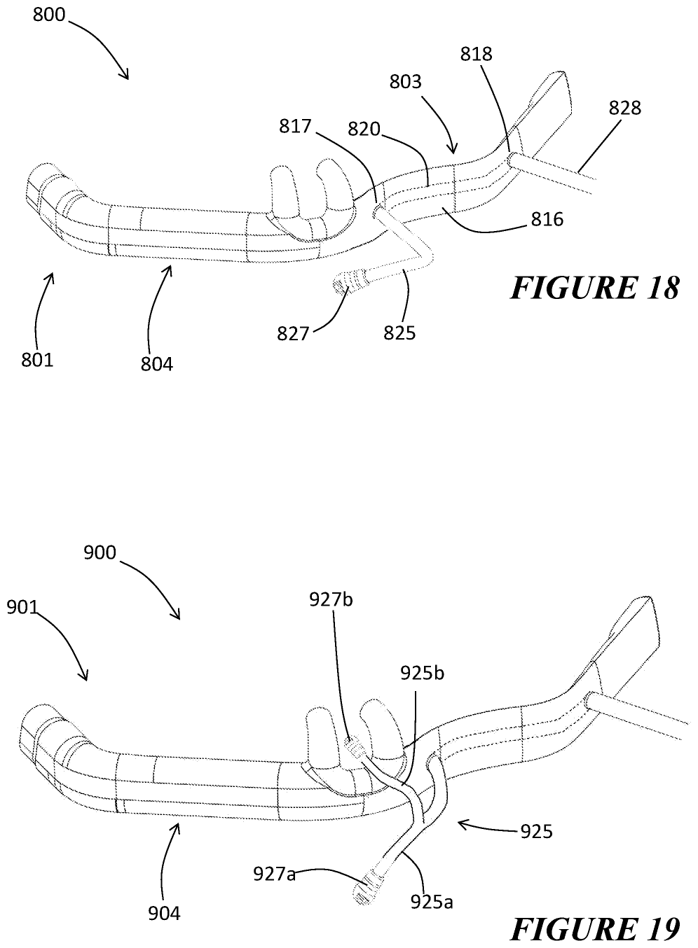

FIG. 18 illustrates a further alternative embodiment in which a patient interface 800 comprises a gases delivery side member 801 comprising a collapsible portion 804. The patient interface 800 includes a non-delivery side member 803 similar in configuration to interface 500 shown in FIG. 11 and in which the non-delivery side member 803 includes pair of spaced apart openings comprising a sampling line inlet 817 and a sampling line outlet 818 formed in the non-patient facing wall 816 and connected by an internal passage 820. An inlet sampling line 825 includes a sampling device 827 and is connected to the sampling line inlet 817 via for example a luer lock, threaded connection, plug fit or barb connection. An outlet sampling line 828 is connected to the sampling line outlet 818 via for example a luer lock, threaded connection, plug fit or barb connection. The outlet sampling line 828 may connect to a respiratory gases monitor (not shown).

The sampling device 827 comprises the sampling inlet for the gas sampling interface. The sampling device 827 is located at an end of the inlet sampling line 825 and is configured for positioning in front of the patient's face and/or around the face and/or inside the patient's mouth. The sampling device 827 could comprise a gas sampling tip equivalent or similar to that which has been previously described by the Applicant in International Patent Publication WO/2018/070885. In an alternative embodiment, the inlet sampling line 825 and outlet sampling line 828 are non-removably connected to the non-delivery side member 803 and could, for example, be moulded to the inlet 817 and outlet 818 respectively.

FIG. 19 illustrates a patient interface 900 which is a variation on the patient interface 800 shown in FIG. 18. The patient interface 900 comprises a gases delivery side member 901 which comprises a collapsible portion 904. The patient interface 900 includes an inlet sampling line 925 which is a wye-piece sampler line and includes a mouth line 925a and a nasal line 925b. A mouth sampling device 927a is located at the end of the mouth line 925a and a nasal sampling device 927b is located at the end of the nasal line 925b. Sampling at both nose and mouth may in some instances provide more reliable capture of patient gases and particularly in instances where it is uncertain where of the nose or mouth the patient is breathing from.

The inlet sampling lines 825, 925 shown in FIGS. 18 and 19 may be malleable to allow for selective positioning of the sampling devices 827, 927a, 927b. The malleability may be configured to require a minimum level of force to manipulate and reposition the sampling lines. This may prevent the sampling devices from undesirable movement which could affect intake of patient gases. The malleability of sampling line 825 in FIG. 18 may allow for the sampling device 827 to be repositioned between the mouth and nose according to need and/or moved out of the way to provide access for other medical instruments. One or more of the sampling lines may be made from a variety of suitable materials such as silicone, pvc (polyvinyl chloride), thermoplastics etc.

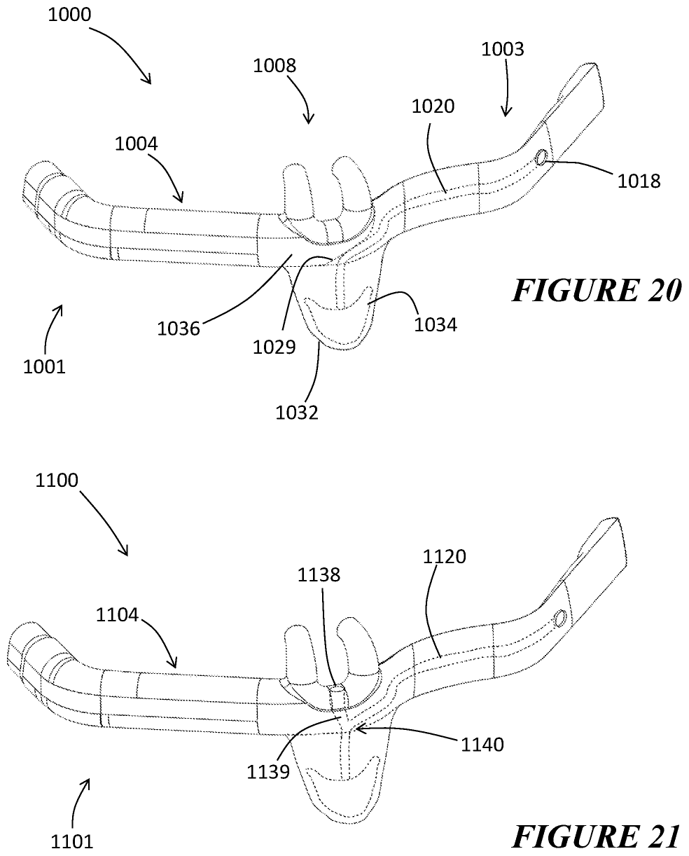

FIG. 20 illustrates another alternative embodiment in which a patient interface 1000 includes a mouth sampling scoop 1032 which includes a scoop opening 1034 configured for location in front of the patient's mouth. Patient gases exhaled through the mouth are captured by the scoop opening 1034 and taken through an internal sampling conduit 1020 which extends through the scoop 1032 and through non-delivery side member 1003 to a sampling outlet port 1018 which is configured for fluid communication with a respiratory gas monitor e.g. via a sampling outlet line. In this regard, the gases sampling interface comprises a mouth scoop 1032 configured to capture patient gases exhaled from the patient. In an alternative embodiment (not illustrated) the mouth scoop 1032 is configured for removable attachment to the patient interface and optionally to the gases delivery interface. The patient interface 1000 further comprises a gases delivery side member 1001 which comprises a collapsible portion 1004

The scoop 1032 has a substantially flat profile allowing it to fit under a patient mask applied over the top of the interface 1000. The scoop 1032 may be sized to cover a relatively small portion of the mouth in order to enable expiratory gas catchment but also allowing room for other medical equipment. The sampling conduit 1020 extends beneath an internal wall 1029 which separates the scoop 1032 from the gases delivery passage 1036 such that the sampling conduit 1020 does not interfere with the gas delivery passage 1036 providing gases to the nasal delivery prongs 1008. In other embodiments, the sampling conduit could extend within the gases delivery passage 1036. The mouth scoop 1032 may be made from or formed of a soft material allowing it to bend around instruments if necessary. The mouth scoop 1032 could be any suitable shape so as to limit interference with instruments which require mouth access.

FIG. 21 illustrates a patient interface 1100 which is a variation on the interface 1000 shown in FIG. 20. In addition to the features/structure of interface 1000, the patient interface 1100 further includes a septum sampling port 1138 for sampling nasal patient gas flow which is transported through a septum sampling line 1139 extending from a junction 1140 in the sampling conduit 1120. This configuration allows for simultaneous nasal and mouth sampling and thereby improving sampling reliability when it is uncertain where the patient is breathing from. In a particular embodiment, the septum port 1138 receives nasal gas flow directly from the patient. In another embodiment, the septum port 1138 could be connected to a nasal sampling prong or another sampling device such as a malleable sampling line described above with respect to FIGS. 18 and 19. The patient interface 1100 further comprises a gases delivery side member 1101 which comprises a collapsible portion 1104

The configurations shown in FIGS. 20 and 21 may also be advantageous in that the inlets 1034, 1138 are positioned centrally and are substantially aligned with the patient's nose and mouth. For example, the inlets 1034, 1138 lie substantially on a common plane with the patient's nose and mouth and also with the nasal delivery prongs 1008. The central positioning of the mouth and nasal inlets 1034, 1138 may advantageously further reduce the chance of interference with the seal of a patient mask which is applied to the patient's face.

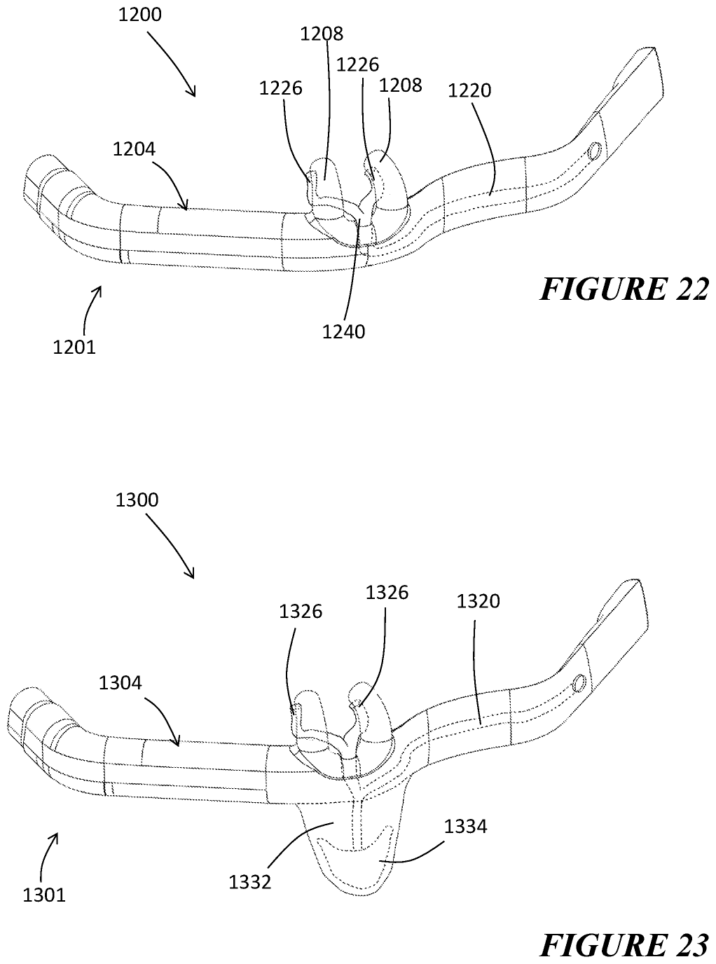

FIG. 22 illustrates another embodiment patient interface 1200 similar to the preceding embodiments but in which the sampling conduit 1220 connects via a wye-piece 1240 to a pair of nasal sampling prongs 1226 positioned alongside and substantially parallel to (and also beneath) the nasal delivery prongs 1208. The nasal sampling prongs could be malleable to allow better positioning or engagement with a patient's nares. The patient interface 1200 further comprises a gases delivery side member 1201 which comprises a collapsible portion 1204

The patient interface 1200 could also be provided with a mouth sampling scoop 1332 as is shown in FIG. 23 which illustrates a patient interface 1300. The scoop 1332 may have an equivalent configuration to that which is described above with respect to scoop 1032 in FIG. 20. The patient interface 1300 includes a sampling conduit 1320 which is therefore capable of sampling patient gas flow from each of the patient's nares via the pair of nasal prongs 1326 as well as from the patient's mouth via the mouth scoop 1332 and its opening 1334. Accordingly, the patient interface 1200 provides a sampling inlet which comprises both a nasal inlet via the nasal prongs 1326 and a mouth inlet via the mouth scoop 1032. The patient interface 1300 further comprises a gases delivery side member 1301 which comprises a collapsible portion 1304

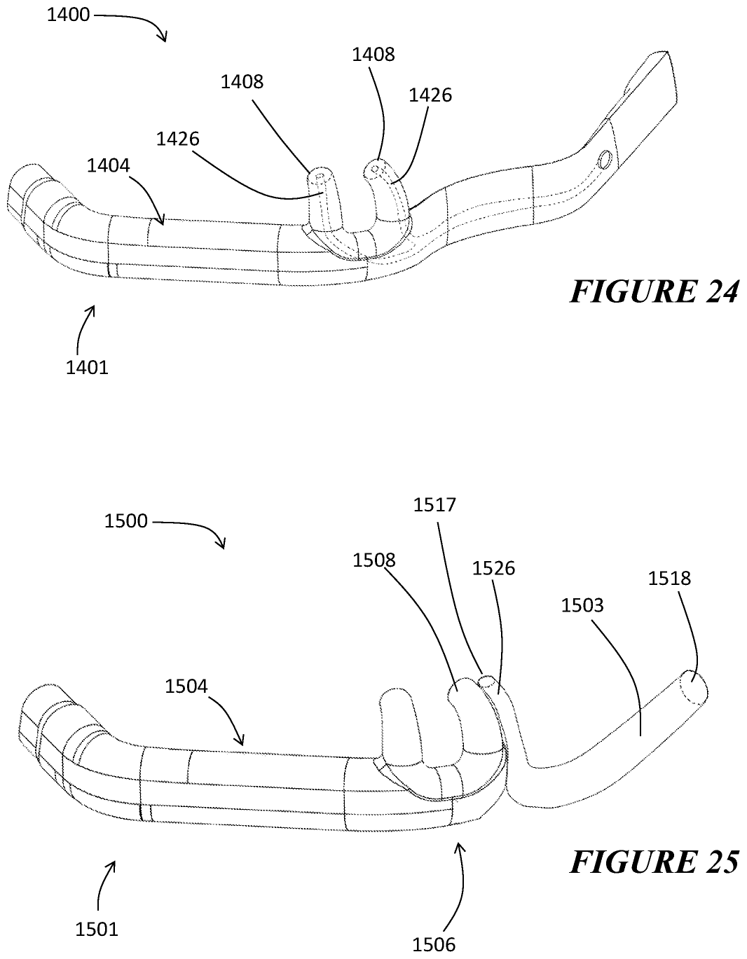

FIG. 24 illustrates a further embodiment patient interface 1400 in which the nasal sampling prongs 1426 extend through the nasal delivery prongs 1408. The nasal sampling prongs 1426 are approximately concentric with the nasal delivery prongs 1408. The nasal sampling prongs 1426 are illustrated terminating at approximately the same point as the nasal delivery prongs 1408 but could also extend beyond the end of the nasal delivery prongs as is exemplified in an alternative embodiment below in FIG. 51. In other embodiments, the nasal sampling prongs could extend through the nasal delivery prongs but not centrally or concentrically. For example, the nasal sampling prongs could be attached to an internal surface of the nasal delivery prongs and therefore offset from the centre of the nasal delivery prongs.

The patient interface 1400 further comprises a gases delivery side member 1401 which comprises a collapsible portion 1404

FIG. 25 illustrates an alternative patient interface 1500 in which the non-delivery side member and the sampling conduit is provided by a sampling tube 1503 having at one end a nasal prong 1526 which provides the sampling inlet 1517 and, at the opposite end, an opening which provides the sampling outlet 1518. The nasal sampling prong 1526 is positioned alongside and extends parallel to one of the nasal delivery prongs 1508. The sampling tube 1503 could be connected to a headstrap (not shown). The sampling tube 1503 could be removable or non-removably connected to the manifold 1506 and/or to the nasal delivery prong 1508. In the illustrated embodiment provided by FIG. 25, the nasal sampling prong 1526 is moulded with the manifold 1506 and also to the nasal delivery prong 1508. The patient interface 1500 further comprises a gases delivery side member 1501 which comprises a collapsible portion 1504

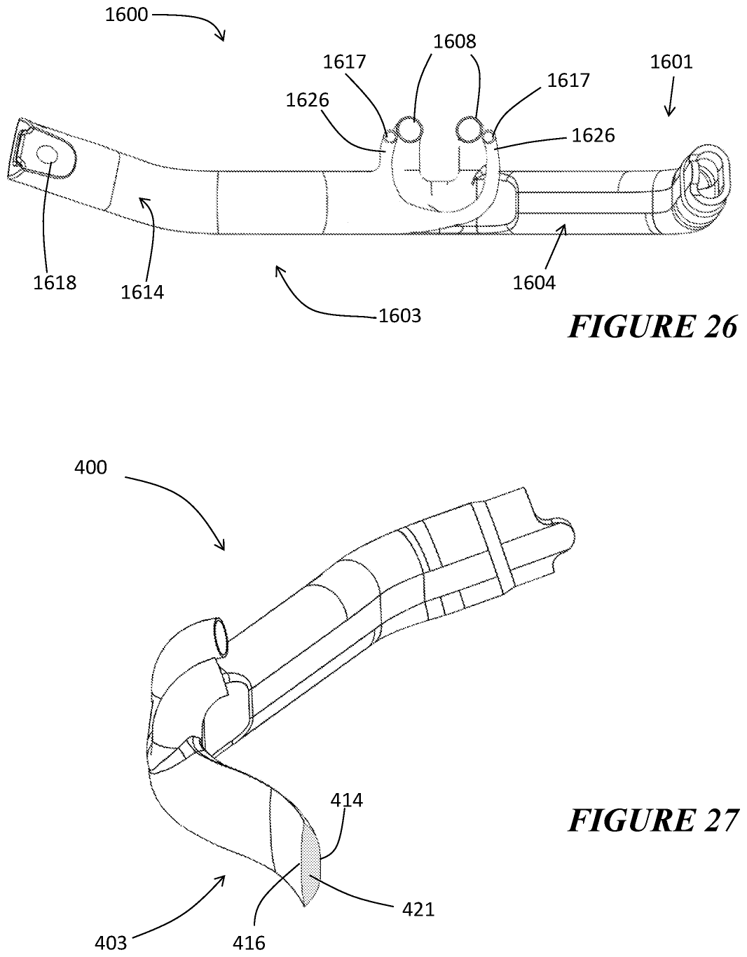

A variation to patient interface 1500 is shown in FIG. 26 in which a patient interface 1600 includes a pair of nasal sampling prongs 1626 attached (e.g. via moulding, adhesive, etc) to the nasal delivery prongs 1608. The nasal sampling prongs comprise a sampling inlet 1617 in fluid communication with a sampling outlet port 1618 which is positioned on a patient-facing surface 1614 of the non-delivery side member 1603 and which is configured for connection to a respiratory gas monitor via an outlet tube or the like. The patient interface 1600 further comprises a gases delivery side member 1601 which comprises a collapsible portion 1604

FIG. 27 provides a side perspective of the patient interface 400 previously shown in FIG. 7 and with the non-delivery side member 403 cut-away to show the cross section 421. The cross-section 421 is equivalent to that which is described above with reference to FIG. 15 and, in particular, is asymmetric and having a patient-facing wall 414 which is curved and a non-patient-facing wall 416 which is less curved and substantially planar.

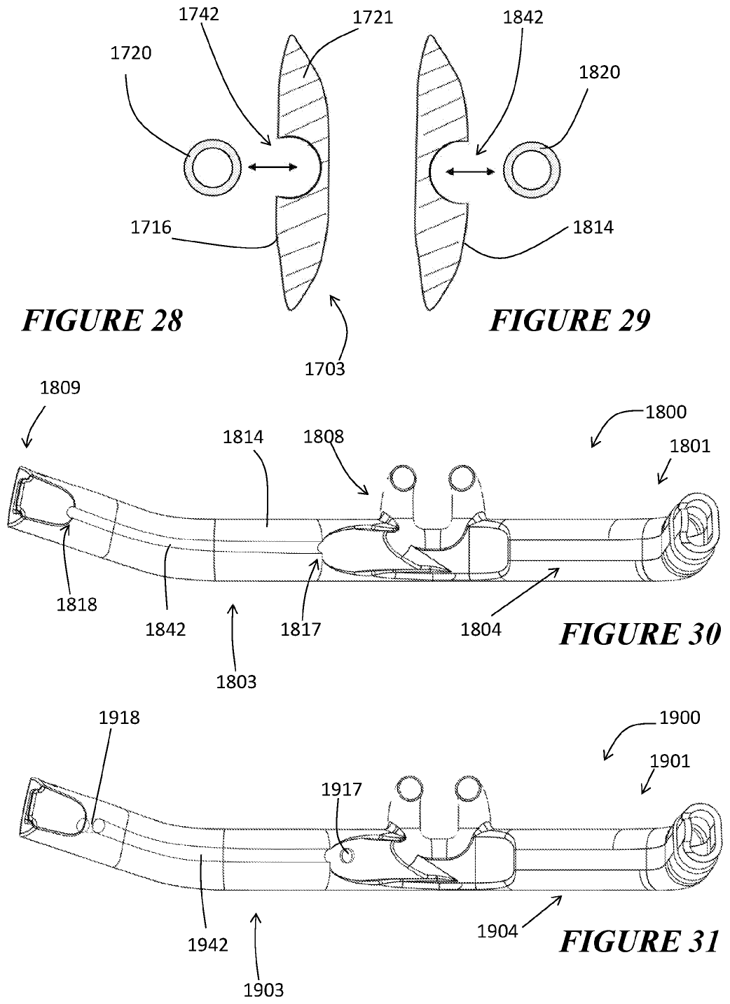

FIG. 27 provides contextual reference for FIGS. 28 to 31 which illustrate various embodiments of the non-delivery side member being provided with a channel configured to receive the sampling conduit.

FIG. 28 illustrates a cross-section 1721 of the non-delivery side member 1703 in which a channel 1742 is provided in the non-patient-facing wall 1716. The channel 1742 is substantially circular and is configured in diameter to receive and retain a tubular sampling conduit 1720. The conduit 1720 is retained in the channel 1742 (e.g. via a snap-fit arrangement) whereby the sampling conduit and/or the channel 1742 is flexibly resilient such that the conduit 1720 is retained within the channel 1742 by slight resilient deformation of the conduit 1720 and/or the channel 1742. In configurations where the sampling conduit 1720 has a non-circular cross-section, the channel 1742 or a part thereof may comprise a cross-sectional shape that matches the cross-sectional shape of the sampling conduit 1720 in order to receive and optionally retain it. In other configurations, the channel may be shallower than what is illustrated in FIGS. 28 and 29 and could comprise a circular or curved cross-section that is less than 180° around. In some configurations, the conduit 1720 is retained within the channel 1742 by a retention mechanism, e.g. adhesive, hook-and-loop configuration, etc.

The channel 1742 may be sufficiently deep so as to receive most or all of the diameter of the conduit 1720 and in order that the periphery of the non-delivery side member cross-section 1721 is not substantially increased or altered when the sampling conduit is fitted within the channel 1742. This may advantageously minimise disruption to the mask seal when placed over the top of the patient interface.

FIG. 29 provides an alternative configuration in which the channel 1842 is located in the patient-facing-wall 1814 and therefore the conduit 1820 is located at the patient-facing wall 1814 instead of the non-patient-facing wall 1716 as in FIG. 28.

FIG. 30 provides a rear perspective of a patient interface 1800 having a non-delivery side member 1803 with the cross-section that was illustrated in FIG. 29. FIG. 30 illustrates the channel 1842 formed in the patient-facing wall 1814 and extending along a length of the non-delivery side member between a sampling inlet end 1817 adjacent to the delivery outlet 1808 and a sampling outlet end 1818 adjacent to a headstrap end 1809 of the non-delivery side member 1803.

A variation to patient interface 1800 is shown in FIG. 31 in which a patient interface 1900 includes a non-delivery side member 1903 having a sampling conduit outlet aperture 1918 and a sampling conduit inlet aperture 1917. The inlet and outlet apertures 1917, 1918 are provided at opposite ends of the channel 1942 for the sampling conduit to be inserted through in order to hold the sampling conduit securely in place. The inlet and outlet apertures 1917, 1918 extend through a portion of the non-delivery side arm 1903 so as to effectively provide connection loops through which the sampling conduit in inserted to provide additional securing of the conduit within the channel 1942. The patient interface 1800 further comprises a gases delivery side member 1801 which comprises a collapsible portion 1804

The embodiments exemplified in FIGS. 11 to 31 illustrate configurations in which the sampling conduit is configured to remain open during application of the collapsing force to the patient interface which induces movement of the collapsible portion to the collapsed configuration. This has usability benefits. For example, this may allow a user to continue monitoring a gas at the patient when a mask 302 is applied over the patient interface 500 without having to disconnect the gas sampling interface 515 from a respiratory gas monitor (e.g. capnography device) and reconnecting the respiratory gas monitor to another sampling interface (for example to a sampling port (not shown) in the mask 302. Additionally or alternatively, this may allow a user to monitor output from a single respiratory gas monitor rather than several monitors connected to various sampling interfaces (for example the gas sampling interface 515 and a sampling port on the mask 302), which could be confusing and thereby increase the risk of incorrect or inaccurate measurements. For example, the sampling conduits illustrated in FIGS. 11 to 24 and 26 may be configured to remain open by virtue of being located internally of the non-delivery side member which may be sufficiently resistant to deformation so as not to allow collapse of the internal sampling conduit during application of the collapsing force. These sampling conduits may be less collapsible than the collapsible portion of the patient interface. The sampling conduits could include internal stiffening or support portions configured to prevent deformation, restriction or closure of the sampling conduit upon application of the collapsing force. Along a common plane or section, the sampling conduits may have a thicker wall than the collapsible portion of the patient interface. Along a common plane or section, the sampling conduits may have a wall of uniform thickness and the collapsible portion may have a wall of non-uniform thickness The sampling conduit exemplified in FIG. 25 is itself the non-delivery side member and in which the sampling tube 1503 could have a sufficiently rigid configuration (for example, provided by the material or geometry of the tube 1503) so as to remain open upon application of the collapsing force. Similarly, the sampling conduits illustrated in FIGS. 28-31 may be only partially surrounded and protected by the non-delivery side member and so may also have a configuration (e.g. in material or geometry or via internal support formations) which allows the sampling conduit to remain open during application of the collapsing force.

The preceding description of FIGS. 11 to 31 related to embodiments of the present disclosure in which the gas sampling interface is provided to (or in lieu of) the non-delivery side member 403. The following description of FIGS. 32 to 65 relate to embodiments in which the gas sampling interface is provided to the gases delivery side member 401 (illustrated in FIG. 7).

FIGS. 32-49 illustrate cross-sections of various configurations in which the sampling conduit is provided to the gases delivery side member. The sampling conduit includes a sampling lumen denoted by ‘S’ and the gases delivery side member includes a gases delivery lumen denoted by ‘G’. The cross-sections illustrated in FIGS. 32-49 are taken through the collapsible portion 404 of the gases delivery side member 401.

FIGS. 32, 35, 38, 41, 44 and 47 illustrate the normally open configuration of the collapsible portion in which both the gases delivery lumen G and the sampling lumen S are open. Each of these figures also illustrate embodiments in which the gases delivery lumen G and the sampling lumen S have parallel longitudinal axes. FIGS. 33, 36, 39, 42, 45 and 48 illustrate the collapsible portion in the collapsed configuration and wherein the sampling lumen is configured to remain open. These configurations therefore allow for continuous sampling of patient gases through the sampling lumen even during reduction or cessation of gas flow through the gases delivery lumen due to the collapsible portion being moved to the collapsed configuration. This has usability benefits. For example, this may allow a user to continue monitoring a gas at the patient when the collapsible portion is in the collapsed configuration (e.g. when a mask is placed over the patient interface) without having to disconnect the gas sampling interface from a respiratory gas monitor (e.g. capnography device) and reconnecting the respiratory gas monitor to another sampling interface (for example to a sampling port (not shown) in the mask. Additionally or alternatively, this may allow a user to monitor output from a single respiratory gas monitor rather than several monitors connected to various sampling interfaces (for example the gas sampling interface and a sampling port on the mask), which could be confusing and thereby increase the risk of incorrect or inaccurate measurements.

FIGS. 34, 37, 40, 43, 46, 49 illustrate alternative embodiments in which the collapsible portion is in the collapsed configuration and wherein the sampling lumen is also configured to be closed. It is envisaged that these configurations may be used where sampling is not required when the collapsible portion is moved to the collapsed configuration. For example, where a patient mask is applied to a patient's face which contains its own patient gas sampling system, it may be desirable for the sampling lumen S to be closed in order to prevent gas leakage from inside the patient mask. For example, in particular situations, certain apparatus flow supply devices could interpret the sampled patient gas removed from the system through the sampling conduit as a ‘leak’ which could trigger an alarm. This scenario could be more likely to occur where a relatively low flow rate is being delivered through a patient mask and wherein the sampled flow rate removed through the sampling conduit constitutes a substantial portion (e.g. 20%) of the apparatus gas flow that is delivered.

The alternative configurations of the sampling conduit remaining open or becoming closed (i.e. the alternative configurations shown in FIGS. 33 and 34, FIGS. 36 and 37, FIGS. 39 and 40, FIGS. 42 and 43, FIGS. 45 and 46, FIGS. 48 and 49) may be achieved according to the level of sampling lumen collapse-resistance. This may be due to the stiffness of the sampling lumen. For example, in some embodiments the sampling lumen may be surrounded by material having higher stiffness as compared to the material surrounding the gases delivery lumen. Consequentially, a collapsing force applied to the collapsible portion (and which is transmitted either directly or indirectly to the sampling lumen) can cause the gases delivery lumen to become occluded or closed whilst the sampling lumen remains open. Alternatively, the sampling lumen could be surrounded by material having similar or lower stiffness to that of the gases delivery lumen and in which case the collapsing force applied to the collapsible portion can cause both lumens to become closed.

FIG. 32 illustrates a first configuration in which the sampling lumen S is integrated within a wall 2021 of the gases delivery member collapsible portion 2004. The wall 2021 surrounds the gases delivery lumen G. The wall 2021 is formed of a non-rigid material and is configured in material and/or in geometry to allow movement of opposite wall portions 2021a, 2021b towards and against each other upon movement of the collapsible portion 2004 to the collapsed configuration. The sampling lumen S has a circular cross section and is located adjacent an end of the elongated cross-section of the gases delivery lumen G. The wall 2021 is enlarged at one end so as to accommodate the sampling lumen S therein.

FIGS. 33 and 34 illustrate the collapsible portion 2004 in the collapsed configuration and in which the sampling lumen G has been closed by movement of the wall portions 2021a, 2021b towards and against each other. FIG. 33 illustrates an embodiment in which the sampling lumen S is configured to remain open whilst the collapsible portion 2004 is in the collapsed configuration. FIG. 34 illustrates an alternative embodiment in which the sampling lumen S is configured to also become closed when the collapsible portion 2004 is in the collapsed configuration.

FIG. 35 illustrates an embodiment in which the collapsible portion 2104 extends through the sampling lumen S and the sampling conduit comprises a sleeve 2144 that surrounds the collapsible portion 2104. The sampling lumen S is formed in the volume between the sleeve and the collapsible portion 2104. FIG. 36 illustrates the collapsible portion 2104 in the collapsed configuration such that the gases delivery lumen G is closed but in which a portion of the sampling lumen remains open between opposite external ends of the collapsible portion 2104 and opposite internal ends of the sleeve 2144.

FIG. 37 illustrates an alternative embodiment to FIG. 36 in which the open configuration shown in FIG. 36 is moved to a collapsed configuration and wherein both the sampling lumen S and the gases delivery lumen G are closed.

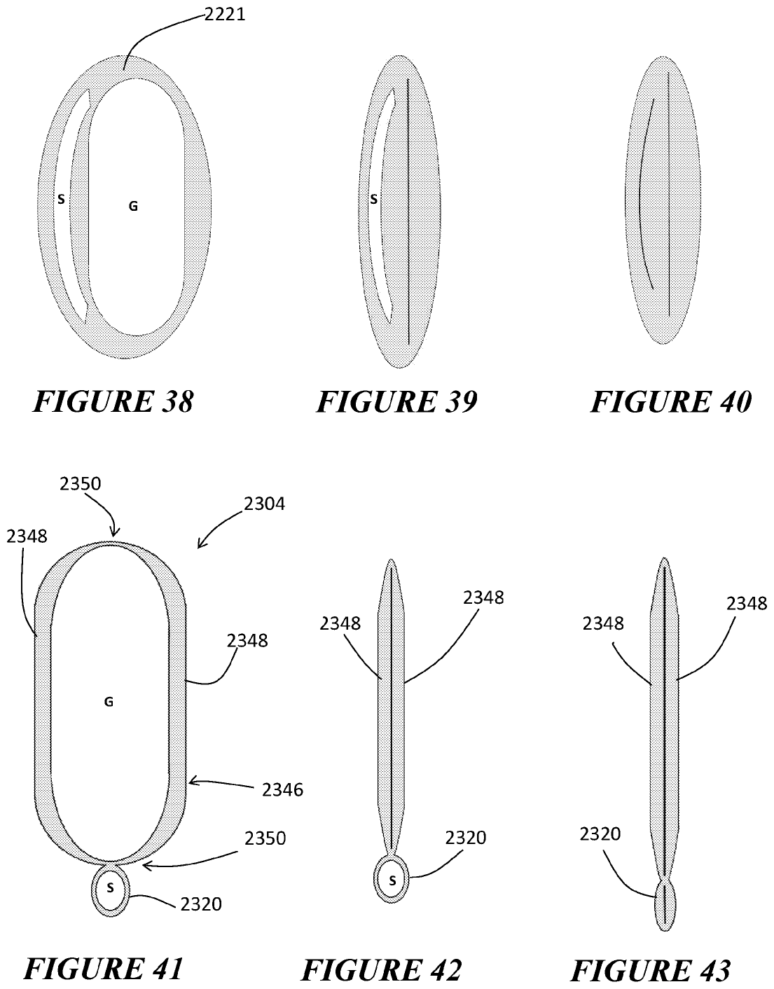

FIG. 38 illustrates an embodiment in which the sampling lumen S and gases delivery lumen S are integrally formed in the gases delivery side member and wherein the sampling lumen S is formed in a wall 2221 which surrounds the gases delivery lumen G. The embodiment of FIG. 38 is therefore a variation to FIG. 32 but differs in that the sampling lumen S of FIG. 38 has an elongated and curved cross-section which extends alongside a longitudinal side of the gases delivery lumen cross-section. The wall 2221 is enlarged at one side so as to accommodate the sampling lumen S therein.

FIG. 39 illustrates an embodiment in which the configuration of FIG. 38 is moved to the collapsed configuration so as to close the gases delivery lumen G but wherein the sampling lumen remains open. FIG. 40 illustrates an alternative embodiment in which the open configuration of FIG. 38 is moved to the collapsed configuration and wherein both the sampling lumen S and the gases delivery lumen G are closed.

FIG. 41 illustrates an embodiment in which a sampling conduit 2320 is integrally formed with the collapsible portion 2304 and whereby the sampling conduit extends alongside an external surface 2346 of the collapsible portion 2304. The collapsible portion 2304 has an elongate cross-section which includes a pair of longitudinal sides 2348 extending between a pair of opposite ends 2350 and the sampling conduit 2320 being integrally connected with the external surface 2346 at one of the ends 2350.

FIG. 42 illustrates an embodiment in which the configuration of FIG. 41 is moved to the collapsed configuration and wherein the longitudinal sides 2348 have moved towards and against one another to close the gases delivery lumen. The sampling conduit 2320 is unaffected and with the sampling lumen S remaining open. FIG. 43 illustrates an alternative embodiment in which the sampling conduit 2320 has also become resiliently deformed to a collapsed configuration during movement of the collapsible portion 2304 to its collapsed configuration and wherein the sampling lumen has also become closed.

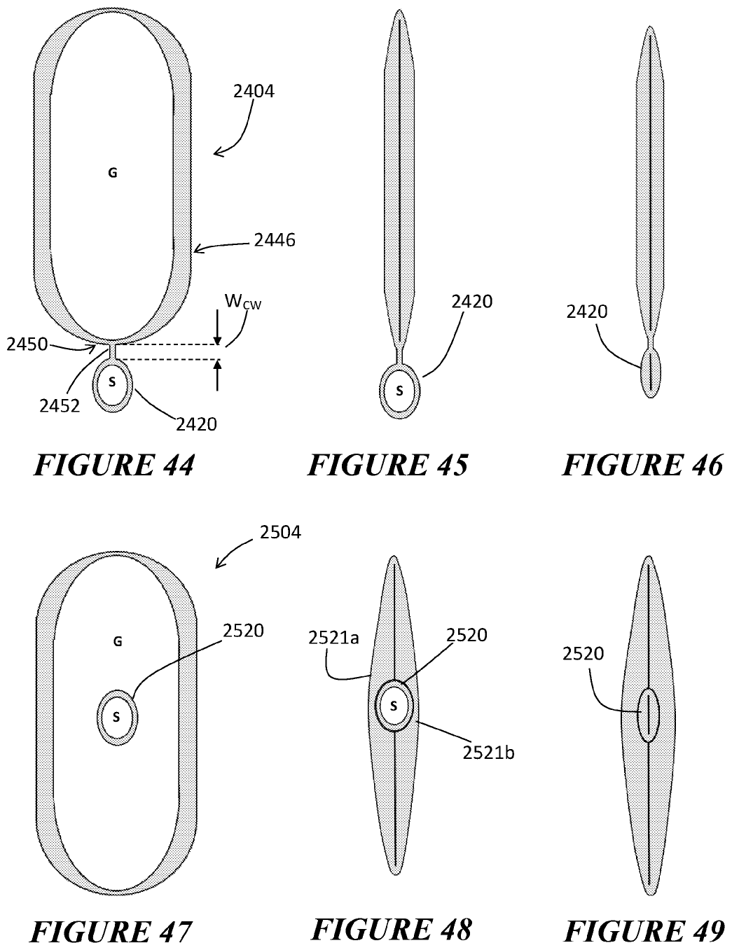

FIG. 44 is a variation on the embodiment shown in FIG. 41 and in which the sampling conduit 2420 is connected with the external surface 2446 (in particular one of the ends 2450) via a connection web 2452 and the sampling conduit 2420 is spaced apart from the collapsible portion 2404 by a width W_{CW} of the connection web 2452.

FIG. 45 illustrates an embodiment in which the open configuration of FIG. 44 is moved to the collapsed configuration and wherein the sampling conduit 2420 remains open. FIG. 46 illustrates an alternative embodiment in which the sampling conduit 2420 is resiliently deformed to a collapsed configuration during movement of the collapsible portion 2404 to the collapsed configuration and wherein the sampling lumen has also become closed.

FIG. 47 illustrates an embodiment in which the sampling conduit 2520 extends through the gases delivery lumen G. The sampling conduit 2520 may be free to move within the gases delivery lumen or could, alternatively, be retained in position via an internal web or retention member (not shown). The sampling conduit 2520 has a small cross-section relative to the gases delivery lumen G so as to minimise obstruction of apparatus gas flow through the gases delivery lumen G and to minimise interference with the moving of the collapsible portion 2504 to the collapsed configuration.

FIG. 48 illustrates an embodiment in which the open configuration of FIG. 47 is moved to the collapsed configuration and in which the sampling conduit 2520 remains open. The sampling conduit 2520 has a curved exterior surface without angular edges which facilitate the wall portions 2521a and 2521b to bend or fold around the sampling conduit 2520. FIG. 49 illustrates an alternative embodiment in which the open configuration of FIG. 47 is moved to the collapsed configuration and in which the sampling conduit 2520 is resiliently deformed to a collapsed configuration such that the sampling lumen is closed.

It will be appreciated that FIGS. 32, 38, 41 and 44 exemplify the sampling conduit (and sampling lumen) being integrated with the gases delivery side member. FIGS. 35 and 47 exemplify the gases delivery lumen and the sampling lumen being substantially concentric and coaxial. FIGS. 32 and 38 illustrate embodiments in which the gases delivery lumen G and the sampling lumen S are integrally formed within the gases delivery side member and spaced apart from one another. FIGS. 41 and 44 illustrate embodiments in which the sampling conduit extends alongside an external surface of the gases delivery side member.

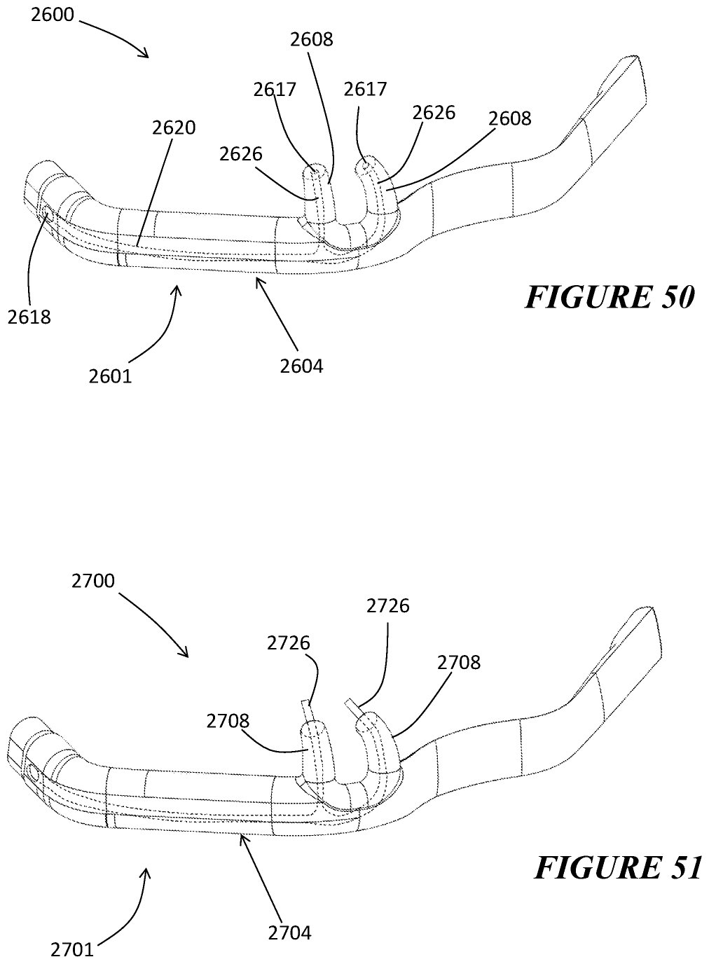

FIG. 50 illustrates a patient interface 2600 in which the gases delivery side member 2601 has the cross-sectional configuration shown in FIG. 47 wherein the sampling conduit 2620 extends through the gases delivery lumen contained within the gases delivery side member 2601. The gases delivery side member 2601 comprises a collapsible portion 2604. The sampling conduit 2620 extends between a sampling outlet comprising an outlet port 2618 and sampling inlets 2617 comprising openings in the ends of nasal sampling prongs 2626 located inside nasal delivery prongs 2608. The sampling conduit 2620 is free to move within the gas delivery side member 2601 and is only fixed to the gases delivery side member 2601 at the outlet port 2618. In other embodiments, the gases delivery side member 2601 comprises internal structures that support the sampling conduit 2620 within the gases delivery side member 2601 at a desired position. The nasal sampling prongs 2626 terminate at the same point as the nasal delivery prongs 2608.

A variation to patient interface 2600 is shown in FIG. 51 in which a patient interface 2700 includes nasal sampling prongs 2726 which extend through and beyond the openings of the nasal delivery prongs 2708. The patient interface 2700 comprises a gases delivery side member 2701 comprising a collapsible portion 2704. The nasal sampling prongs 2726 are therefore configured to position the sampling inlets 2617 further into the patient's nares than the outlets of the nasal delivery prongs 2708. Positioning of the sampling inlets 2617 further inside the nares than the delivery outlet (i.e. the nasal delivery prongs 2708) may provide a number of benefits including minimising dilution of the sampled patient gases. Moreover, this configuration may in some instances minimise the formation of turbulent flow which could restrict or reduce patient gases from entering the sampling prongs 2726. Furthermore, having the sampling prongs 2726 extend beyond the nasal delivery prongs 2708 may shield the patient gases entering the sampling prongs 2726 from the delivered apparatus gases exiting the outlet of the nasal delivery prongs 2708.

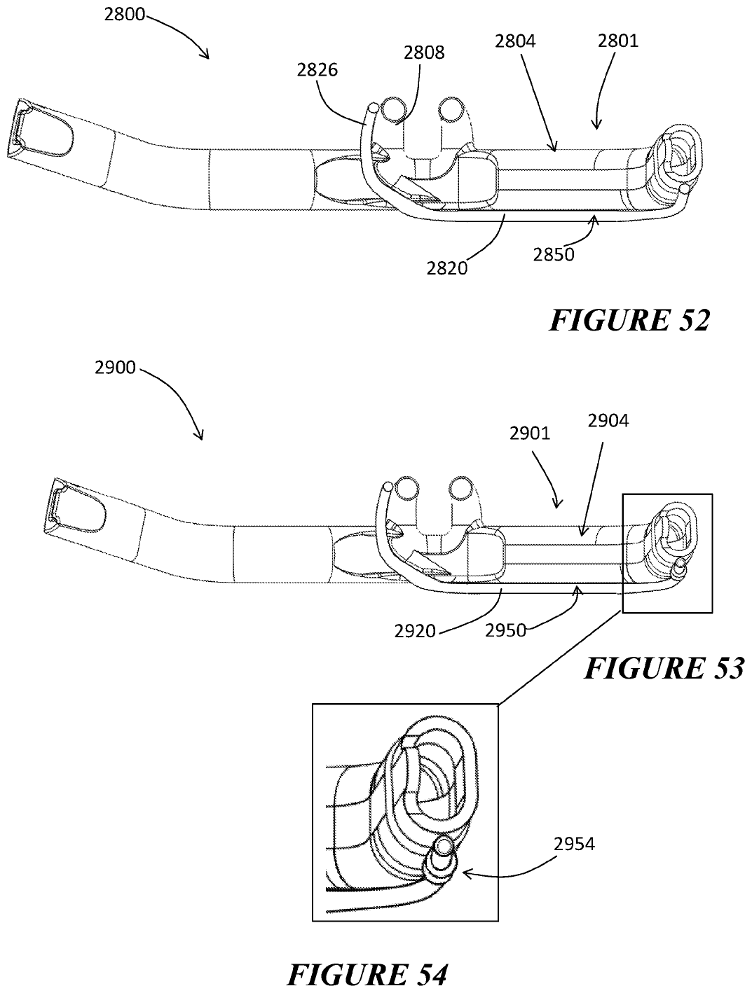

FIG. 52 illustrates a patient interface 2800 in which the gases delivery side member 2801 has the cross-sectional configuration shown in FIG. 41 and wherein the sampling conduit 2820 is integrally formed with the gases delivery side member 2801. In particular, the sampling conduit 2820 is moulded to an underside end 2850 of the gases delivery side member 2801. The sampling conduit 2820 includes a sampling inlet comprising a nasal sampling prong 2826 moulded to (and extending alongside) one of the nasal delivery prongs 2808. The gases delivery side member 2801 comprises a collapsible portion 2804.

A variation to patient interface 2800 is shown in FIGS. 53 and 54 in which a patient interface 2900 includes a sampling conduit 2920 which is removably attached to the underside end 2950 of the gases delivery side member 2901 via an attachment clip 2954. The gases delivery side member 2901 comprises a collapsible portion 2904. In another configuration (not illustrated) the sampling conduit could be similarly attached but located at a top of the gases delivery side member. In an example (not illustrated), the sampling conduit is removably attached to the patient interface at or proximate to the manifold and the remainder of the conduit is separate from but extends along the same side as the gases delivery side member. This may avoid having multiple conduits wrapping around the patient's head which could get in the way of other medical equipment.

The gas sampling interface may be formed of a different material than the collapsible portion. For example (and with reference to FIG. 53), the sampling conduit 2920 may be formed of a different material than the collapsible portion 2904. The sampling conduit 2920 may be formed of a material that has greater material stiffness than a material of the collapsible portion 2904. In a particular embodiment, the sampling conduit 2920 comprises silicone. In an embodiment, the collapsible portion 2904 comprises thermoplastic elastomer. In an embodiment, the sampling conduit 2920 comprises silicone and the collapsible portion 2904 comprises thermoplastic elastomer.

As exemplified in various embodiments and including FIG. 53, the sampling conduit 2920 has a width that is less than a width of the collapsible portion 2904.

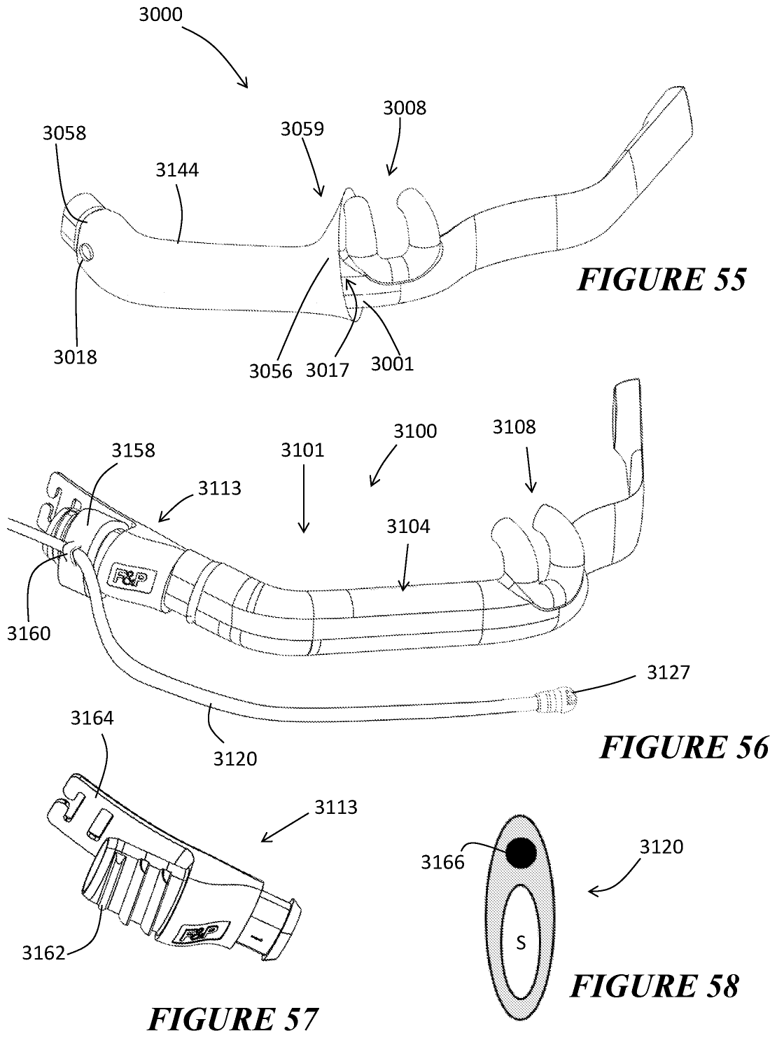

FIG. 55 illustrates a patient interface 3000 in which the gases delivery side member 3001 has the cross-sectional configuration shown in FIG. 35 and wherein the gases delivery side member 3001 is surrounded by a sleeve 3144. The gases delivery side member 3001 comprises a collapsible portion 3104 which is concealed in FIG. 55 by the sleeve 3144. The sleeve 3144 includes an outlet port 3018 configured for connection with a respiratory gas monitor. The sleeve 3144 includes an outlet end 3058 that is sealed with the gases delivery side member 3001. The sleeve 3144 further includes a funnel portion 3056 at an inlet end 3059 of the sleeve proximate to the delivery outlet 3008. The funnel portion 3056 is configured to receive patient gas flow from the nose and/or mouth via an opening 3017 which provides the sampling inlet.

FIG. 56 illustrates a patient interface 3100 in which the gas path connector 3113 is fitted with a ring connector 3158 which includes a loop 3160 to receive and secure part of the sampling line 3120. The ring connector 3158 thereby secures part of the sampling line 3120 to the gas path connector 3113. The sampling line 3120 includes a sampling inlet which is comprised of a sampling device 3127 at a distal end of the sampling line 3120. The sampling line 3120 has a self-supporting and malleable configuration and whereby the sampling device 3127 can be repositioned as required. For example, the sampling device 3127 can be positioned in front of (i.e. on the non-patient side of) the delivery outlet 3108 so as to receive patient gas flow from the patients mouth and/or nose.

FIG. 57 shows the gas path connector 3113 which includes a threaded portion 3162 for connection to a gas supply tube that provides an apparatus gas supply. The gas path connector 3113 also includes a flange 3164 for connection to a head strap. The ring connector 3158 is configured to fit over the threaded portion 3162 (preferably also over the gas supply tube connected to the threaded portion 3162) in order to removably attach the sampling line 3120 to the gas path connector 3113. The ring connector 3158 can comprise a locating feature (e.g. a rib) to resist axial movement with respect to the gas path connector 3113 and gas supply tube when connected.

FIG. 58 shows a cross-sectional view of the sampling line 3120 which includes a flexibly resilient wire 3166 integrated within the sampling line 3120 to provide the sampling line 3120 with its self-supporting and malleable functionality. The sampling line 3120 includes a sampling lumen S. The flexibly resilient wire 3166 is provided in a lumen separate from the sampling lumen S. Both the sampling line 3120 and the sampling lumen S have an elongate cross-section and, in particular, an oval cross-section. The sampling line 3120 is relatively thin in a width direction so as to allow a patient face mask to be placed over the sampling line 3120 and to introduce minimal disruption to the face mask seal.

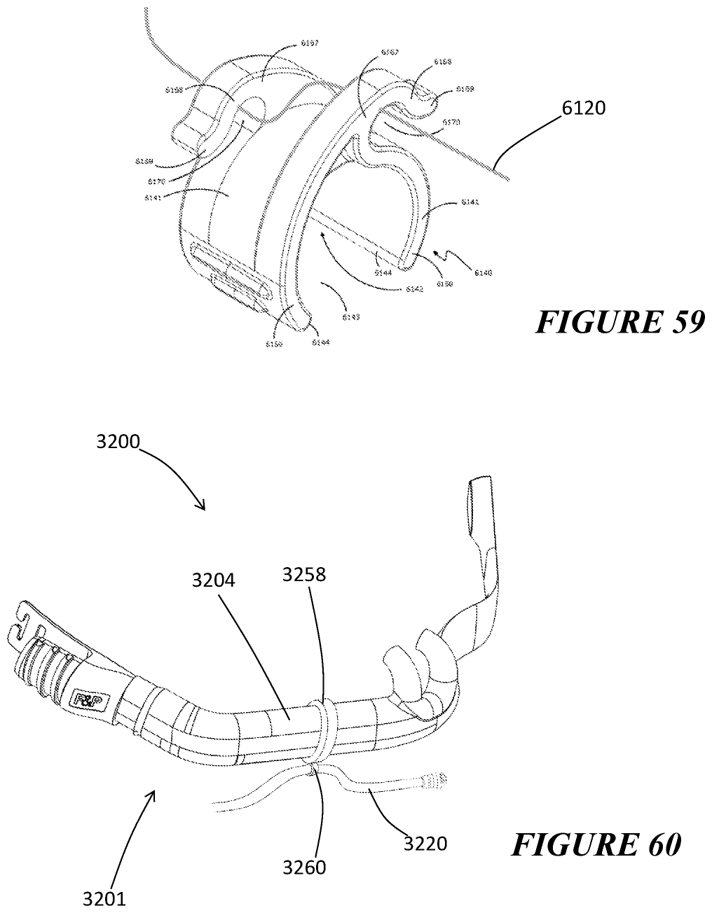

FIG. 59 illustrates an attachment ring 6140 which was previously illustrated in Applicant's earlier patent publication WO2018070885 and which is also suitable for use with a patient interface according to an aspect of the present disclosure. The attachment ring 6140 includes a pair of resilient arms 6150 configured for connection with the gas path connector 3113 shown in FIG. 57. The interior region 6142 of the arms 6150 may have a profile (e.g. a protrusion) to correspond and engage with the threaded portion 3162 of the gas path connector 3113.

The attachment ring 6140 further comprises a pair of clips 6167 forming hooks which comprise arms 6168 extending from the clip body 6141. The clips 6167 provide a concave receiving region 6170 within which a portion of the gas sampling conduit 6120 is received and held. The gas sampling conduit 6120 is threaded through the receiving regions 6170 as shown in FIG. 59 to secure the conduit 6120 in place via a snap-fit connection.

FIG. 60 illustrates a patient interface 3200 in which the gases delivery side member 3201 is fitted with an accessory comprising a ring 3258 that is configured to facilitate moving of the collapsible portion 3204 to the collapsed configuration upon application of the collapsing force. As shown, the ring 3258 is fitted around the collapsible portion 3204. An earlier embodiment of a similar accessory intended to facilitate collapse of a collapsible portion was disclosed in Applicant's International Patent Application PCT/IB2019/051137 (International Patent Publication WO2019159063). FIGS. 25A-F of this disclosure illustrated a ring 215 configured to extend around the conduit and to tilt or rotate when a force is applied to the ring 215 in order to pinch or kink the conduit.

The ring 3258 shown in FIG. 60 of the present disclosure may have a generally equivalent configuration to (and function the same as) the ring 215 disclosed in International Patent Application PCT/IB2019/051137 (International Patent Publication WO2019159063). However, the ring 3258 further includes an attachment loop 3260 for attachment to the sampling conduit 3220. The ring 3258 may therefore serve a dual function to facilitate movement of the collapsible portion to the collapsed configuration and also act as a conduit connector. The application of a face mask over the patient interface can force the ring 3258 to roll over and seal the collapsible potion 3204. The ring 3258 may be sufficiently small so as to minimise any disruption to the seal of the patient face mask.

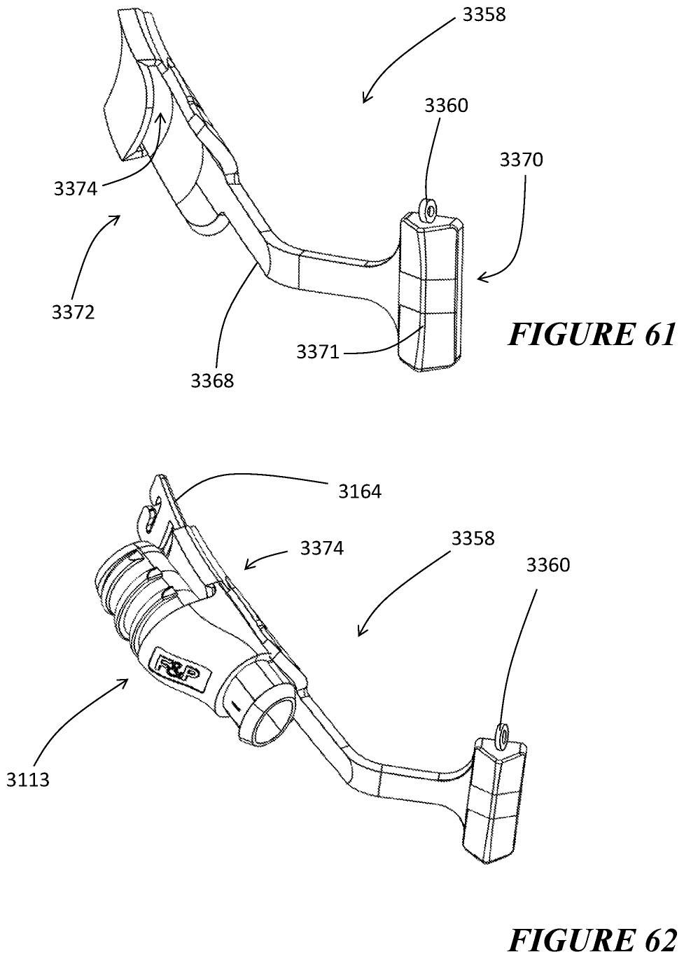

FIG. 61 illustrates an accessory 3358 configured for attachment to a patient interface such as the patient interface 400 illustrated in FIG. 7. The accessory 3358 comprises a rigid member 3368 configured for extending along the patient-facing wall of the gases delivery side member 401. A contact element 3370 is provided at a distal end of the rigid member 3368 and which is configured to provide a concentrated reaction force to a load applied to the collapsible portion 404 shown in FIG. 7. The contact element 3370 is configured to provide the reaction load to a patient-facing wall of the collapsible portion 404 in response to the application of a collapsing force (for example from a patient mask) applied to the non-patient-facing wall of the collapsible portion 404. The contact element 3370 includes a saddle-shaped tapered rib 3371 configured to localise/concentrate the reaction force onto a smaller area of the collapsible portion and thereby further promote bending or folding of the patient-facing wall and thus facilitate movement to the collapsed configuration.

The accessory 3358 further includes an attachment configuration 3372 for attaching the accessory 3358 to the patient interface 400. The attachment configuration 3372 includes an opening 3374 configured to receive and engage with the headstrap flange 3164 on the gas path connector 3113 shown in FIG. 57 (and also shown in FIG. 7). The mode of attachment is shown in FIG. 62 which shows the attachment configuration 3372 attached to the gas path connector 3113 of FIG. 57 and with the headstrap flange 3164 protruding through the opening 3374. In other embodiments, the attachment to the gas path connector could be via a C or U-shaped clip. Other forms of attachment are possible such as adhesive, overmoulding or integrally forming of the accessory with the gas path connector.

The contact element 3370 includes a connection ring 3360 configured for connection with a sampling line such as the sampling line 3220 of FIG. 60 or 3120 of FIG. 56.

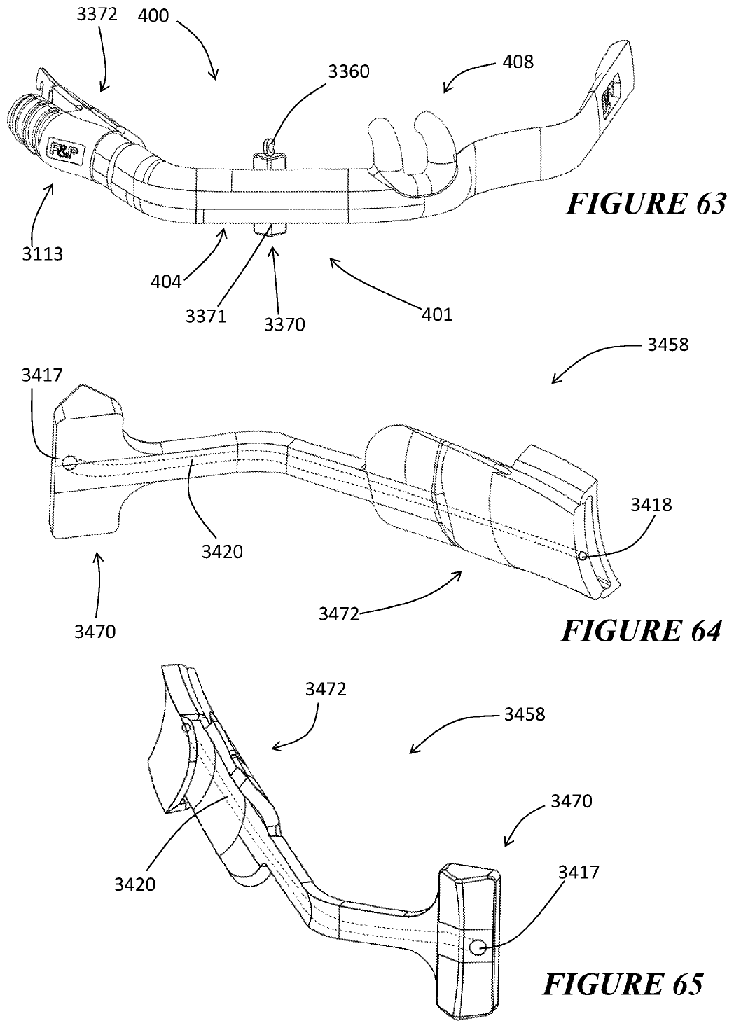

FIG. 63 illustrates the patient interface 400 of FIG. 7 fitted with the accessory 3358. The contact element 3370 is located behind the collapsible portion 404 such that the rib 3371 contacts the patient-facing wall of the collapsible portion 404. The attachment configuration 3372 is connected to the gas path connector 3113 through which apparatus gas flow is provided to the gas delivery side member 401. The attachment ring 3360 is located above (and is spaced from) the collapsible portion 404 to allow for a sampling line to be connected and to run generally along and above the gases delivery side member 401 toward the delivery outlet 408.

FIGS. 64 and 65 are rear and front views of an alternative accessory 3458 which is equivalent in condition to accessory 3358 shown in FIGS. 61-63 except accessory 3458 does not include the attachment ring 3360 and instead includes an integrated sampling conduit 3420 extending internally through the accessory 3458. The sampling conduit 3420 extends between a pair of openings located at opposite ends of the accessory 3458 and comprising a sampling inlet 3417 provided in the contact element 3470 and a sampling outlet 3418 provided at the attachment configuration 3472.

The sampling inlet 3417 may be configured for connection to a sampling device or sampling line. For example, via a luer lock, threaded connection, plug fit, barb fit. The sampling inlet 3417 may be integrally connected, for example moulded, to a sampling device or sampling line. The sampling outlet 3418 may be configured for connection with or may be integrally connected with an outlet line in fluid communication with a respiratory gas monitor. The sampling conduit 3420 is formed within the rigid material of the accessory 3458 and is therefore prevented from collapse during collapse of the collapsible portion thus enabling sampling to continue when apparatus gas flow through the collapsible portion is reduced or stopped.

Various embodiments of a patient interface are described above with reference to the accompanying Figures. It will be appreciated that the patient interface comprises a gases delivery interface which includes an apparatus gases flow path configured to provide apparatus gases to a patient. According to a particular embodiment, in the normally open configuration, the gases delivery interface is configured to allow an apparatus gas flow rate of about 20 L/min to about 90 L/min through the apparatus gases flow path. By way of example, with reference to the patient interface 500 illustrated in FIG. 11, the gases delivery side member 501 is configured to allow an apparatus gas flow rate of about 20 L/min to about 90 L/min through an apparatus gases flow path within the gases delivery side member 501.

In a particular embodiment, when the collapsible portion 504 is moved to the collapsed configuration, the patient interface 500 is configured to allow an apparatus gas flow rate through the apparatus gas flow path of the gases delivery member 501 that is at least 20 times greater than a patient gas flow rate through the gas sampling interface 515. For example, a flow rate through the gas sampling interface 515 (and through the sampling conduit 520) may be less than about 500 ml/min and a flow rate through the gases delivery member 501 when the collapsible portion 504 is in the closed configuration, may be less than about 10 L/min.

In a particular embodiment, when the collapsible portion 504 is in the collapsed configuration, the gases delivery member 501 is configured to allow an apparatus gas flow rate of less than about 10 L/min through the apparatus gases flow path and the gas sampling interface 515 is configured to allow a patient gas flow rate of less than about 500 mL/min, optionally about 40 mL/min to about 500 mL/min through the gas sampling interface 515.

It will be appreciated from the various illustrated embodiments that the patient interface can comprise a single sampling conduit. The provision of a single conduit only may advantageously minimise the number of conduits associated with the patient interface.

The sampling conduit 520 of FIG. 11 (as with various alternative embodiment sampling conduits shown in other Figures) comprises a single lumen and that single lumen is a sampling lumen for the patient gas flow. The sampling conduit 520 does not include any additional lumens other than the sampling lumen. In contrast to the embodiment illustrated in FIGS. 56 and 58, the sampling conduit 520 of FIG. 11 does not comprise a support member or support wire and therefore the sampling conduit 520 does not require or comprise a lumen for a support wire. The provision of a single lumen (i.e. the sampling lumen) may advantageously simplify manufacturing and reduce cost.

### Patient Interface and Accessory Therefor

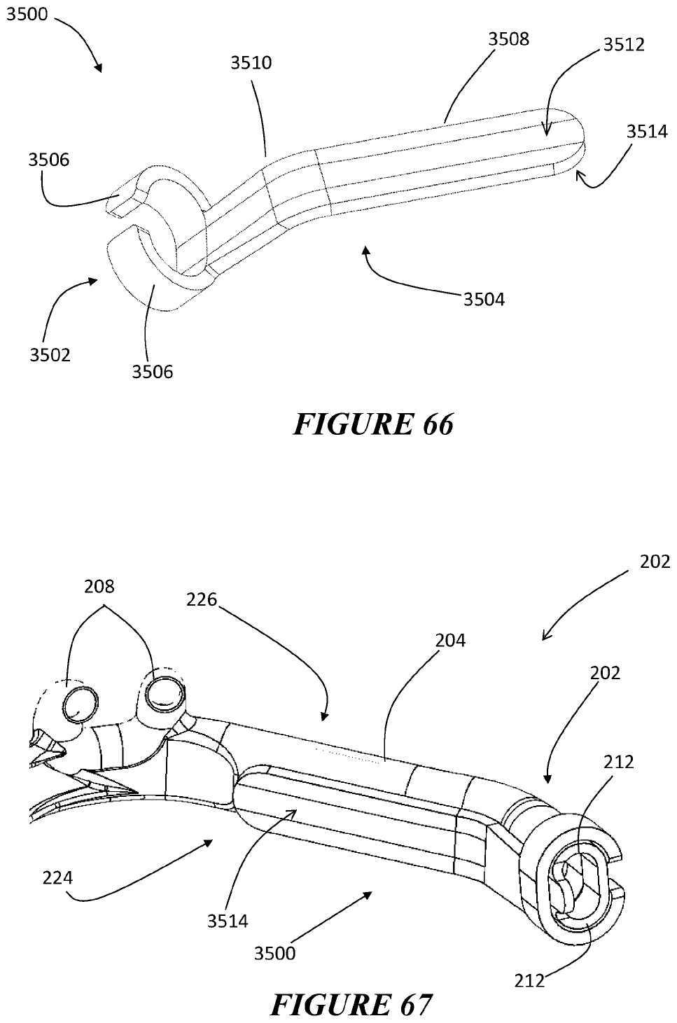

FIGS. 66 to 108 show non-limiting exemplary embodiments of an accessory for a patient interface configured to deliver respiratory gases to a patient via a gases delivery conduit which includes a collapsible portion. For example, the accessory according to one of the embodiments shown in FIGS. 66 to 108 may be used with the collapsible first portion 204 of the patient interface 200 as described above and illustrated in FIGS. 2 to 4.

As noted, the first portion 204 of the patient interface 200 is configured to be collapsible and will hereinafter be referred to as collapsible portion 204. The accessory according to the various embodiments is configured to facilitate or otherwise enhance or promote collapse of the collapsible portion 204 in order to reduce or stop respiratory gas flow through the patient interface 200. In an example, the accessory according to the various embodiments is configured to facilitate or otherwise enhance or promote collapse of the collapsible portion 204 in order to reduce or stop respiratory gas flow to an outlet of the patient interface 200, e.g. prongs 208. The various accessory embodiments shown in FIGS. 66 to 108 will now be discussed in further detail.

With reference to FIG. 66 there is illustrated an accessory 3500 including an attachment configuration comprising a clip 3502 and a backing plate 3504 extending from the clip 3502.

The clip 3502 comprises a flexibly resilient c-clip configured to attach onto a portion of the patient interface 200. For example, the clip 3502 may attach to the gas conduit 202 of the patient interface 200 or to a gas connector. In an embodiment, the clip couples with a rigid support for increased stability. For example, the clip may couple with a rigid support of a gas connector. The clip 3502 includes a pair of flexibly resilient clip arms 3506 which are pre-formed with a curve conforming to the opposite curved sides 212 of the gas conduit 202, as best shown in FIG. 67.

Returning to FIG. 66, the backing plate 3504 includes a pre-formed curve conforming with a contour in the face of the patient P and/or in the patient interface 200. The backing plate 3504 includes an elongate straight portion 3508, and a curved portion 3510 between the clip 3502 and the straight portion 3508. In some embodiments, the straight portion 3508 is substantially planar. The straight portion 3508 includes a rigid contact surface 3512 configured to, in use, face the gas conduit 202. The straight portion 3508 includes a patient-facing surface 3514 at an opposite side of the backing plate 3504 from the contact surface 3512 and which is best shown in FIG. 67.

Turning to FIG. 67, there is illustrated a section of the patient interface 200 in which the gas conduit 202, collapsible portion 204 and nasal prongs 208 are illustrated. The gas conduit 202 and collapsible portion 204 have a patient-facing side 224 which may also be termed an ‘inner’ side and a non-patient-facing side 226 which may also be termed an ‘outer’ side, with the terms inner and outer understood in spatial relation to the patient's face. FIG. 67 is a perspective view looking toward the patient-facing side 224. The accessory 3500 is located at the patient-facing side 224 and would therefore, in use, be positioned between patient's face and collapsible portion 202.

In use, the patient-facing surface 3514 of the straight portion 3508 faces toward (and may typically also be in contact with) the patient's face. As shown in FIG. 67, the contact surface 3512 (shown in FIG. 66 and concealed in FIG. 67) faces toward and contacts a patient-facing surface of the collapsible portion 204 which is concealed behind the straight portion 3514 in FIG. 67.

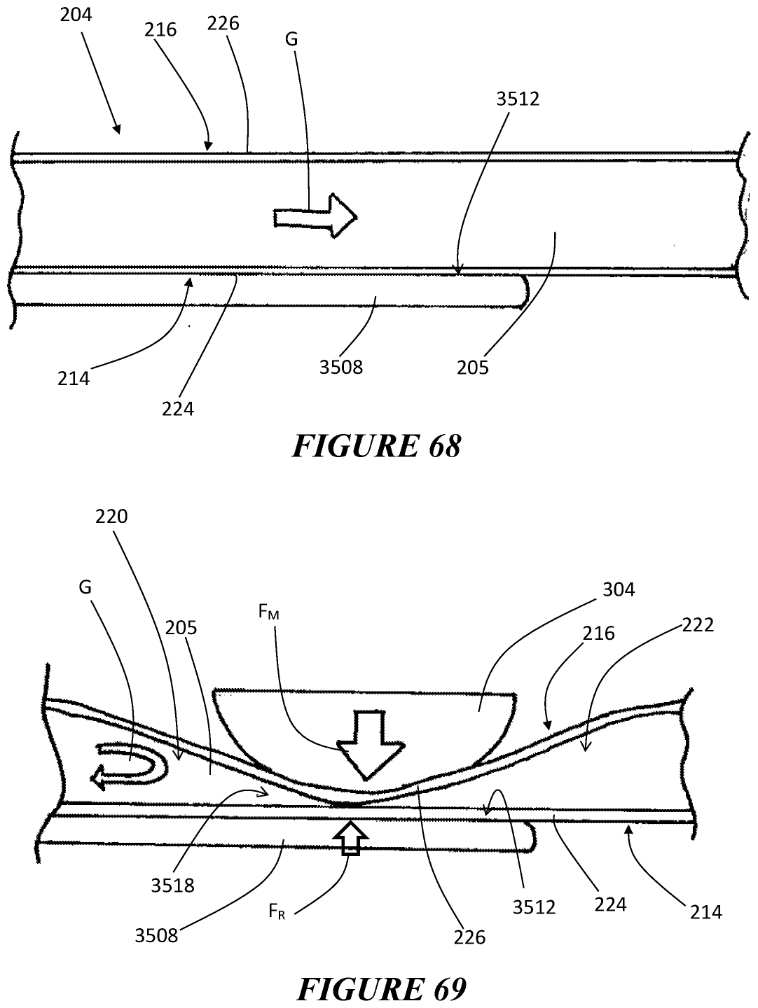

The contact surface 3512 defines a contact portion of the accessory which, in use, contacts the collapsible portion 204 and provides a rigid supporting surface between the collapsible portion 204 and the patient's face. The contact surface 3512 facilitates collapse of the collapsible portion 204 when a collapsing force is applied to the collapsible portion 204. For example, a collapsing force applied by a face mask which is overlaid across the collapsible portion 204 as shown in FIG. 3. This functionality will be further described with reference to FIGS. 68 and 9 which provide side sectional perspectives of the arrangement shown in FIG. 67 in both an uncollapsed (FIG. 68) and a collapsed configuration (FIG. 69).

FIG. 68 shows the collapsible portion 204 overlying the backing plate straight portion 3508. The collapsible portion 204 includes a patient-side 224 and a non-patient-side 226 opposite to the patient side 226. The patient-side 224 includes a patient-facing-surface 214 which, in use, faces generally toward the patient's face. The non-patient-side 226 includes a non-patient-facing surface 216 which, in use, faces generally outwardly from the patient's face.

As shown in FIG. 68, the patient-facing surface 214 overlies and is in contact with the contact surface 3512 of the backing plate straight portion 3508. The interior of the collapsible portion 204 defines a lumen 205 providing passage for respiratory gases G flowing through the gas conduit 202. FIG. 68 illustrates an uncollapsed configuration in which the collapsible portion is open and unobstructed allowing high flow respiratory gases G to pass through lumen 205 in a downstream direction toward the patient.

Turning to FIG. 69, there is illustrated a collapsed configuration whereby a seal 304 (for example an inflatable cuff) of a patient mask is overlaid and pressed down onto the non-patient-facing surface 216 of collapsible portion 204. The applied force FM onto the non-patient-facing surface 216 collapses the non-patient-side 226 inward toward the backing plate 3508 and into contact with the patient-side 224 creating an obstruction 3518 which occludes or obstructs lumen 205 and thereby reducing or preventing flow of respiratory gases G towards the patient. The applied mask force FM produces a substantially equivalent reaction force FR applied to the patient-facing-surface 214 by the backing plate straight portion 3508.

In some configurations the flow of respiratory gases G towards the patient may be completely prevented. In other configurations there may remain a residual flow of respiratory gases from an upstream side 220 of the obstruction 3518 to a downstream side 222 of the obstruction 3518. In instances where a residual flow traverses the obstruction 3518 the total flow rate through the collapsible portion 204 is still significantly reduced as compared to the uncollapsed configuration shown in FIG. 68.

As shown in FIG. 69, the patient-facing surface of the collapsible portion 204 is supported by the contact surface 3512 and is preferably unmoved during collapse of the collapsible portion. In this manner, the contact surface 3512 provides a rigid support surface against which the non-patient-facing surface 216 is pressed against when the force of the mask seal 304 is applied to the collapsible portion 204. The backing plate 3508 thereby facilitates collapse of the collapsible portion 204 insofar as it provides an abutment against which the collapsible portion 204 or a portion thereof is compressed, thereby deforming the collapsible portion 204 into the collapsed configuration shown in FIG. 69.

The accessory 3500 illustrated in FIGS. 66 and 67 and operatively in FIGS. 68 and 69 therefore provides a rigid support surface behind the collapsible portion 204 and at an appropriate location along the patient interface to facilitate collapse of the collapsible portion. The backing plate 3508 is locatable at the desired location of collapse in order to provide the reaction force FR which cooperates with the mask force FM to cause collapse of the collapsible portion 204.

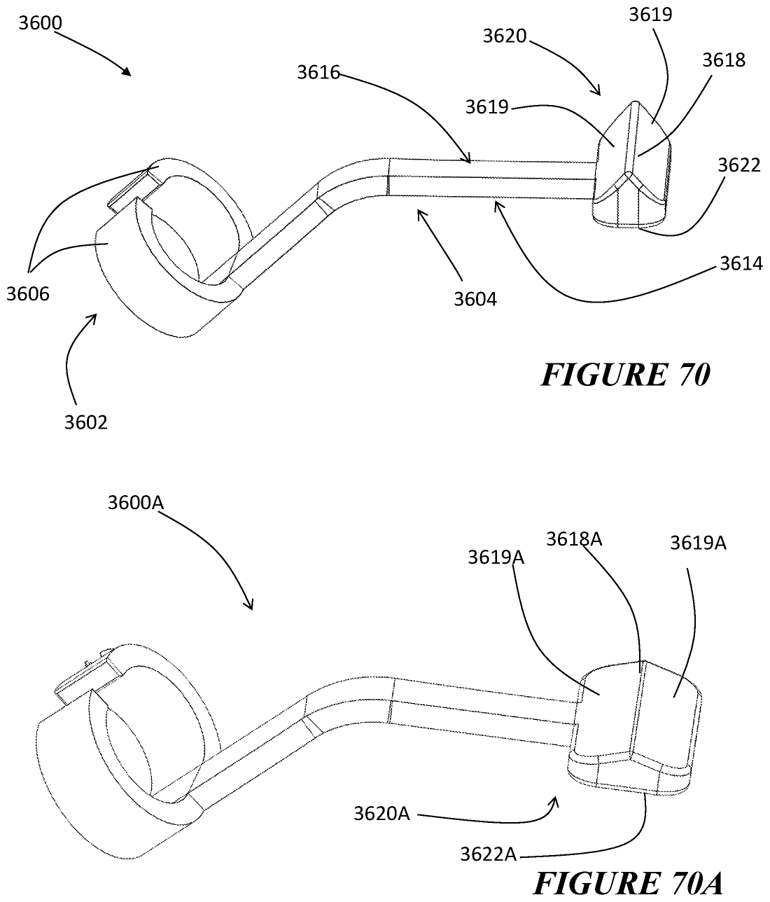

Turning to FIG. 70, there is illustrated an alternative embodiment of an accessory 3600 configured for location between the patient's face and the collapsible portion 204 of the patient interface 200. The accessory 3600 includes a patient-facing-surface 3614 which, in use, is faced toward and/or is in contact with the patient's face. The accessory 3600 includes a non-patient-facing surface 3616 which, in use, is faced away or outwardly from the patient's face.

The accessory 3600 includes a C-shaped clip 3602 which is equivalent to clip 3502 in the preceding embodiment. The clip 3602 includes a pair of resilient clip arms 3606 extending, in use, away from the patient's face. Clip 3602 allows removable attachment of accessory 3600 with the patient interface 200. The accessory 3600 includes a support member 3604 extending between the clip 3602 and a contact element 3616. The contact element 3620 has an elongate profile orientated generally perpendicular to the support member 3604. The contact element includes a flat base 3622 defining a portion of the patient-facing surface 3614. A tapered rib 3618 is located on an opposite side of the contact portion 3620 from the flat base 3622. The tapered rib 3618 extends a non-patient-facing direction and, in use, extends outwardly from the patient's face. In particular embodiments, the rib may not necessarily be tapered and may have a non-tapered configuration such as generally flat contact surface.

The support member 3604 comprises an elongate bar with a pre-formed curve to conform the support member 3604 with a contour of the patient's face and/or of the gas delivery conduit 202 shown in FIGS. 2 and 3.

FIG. 70A illustrates an accessory 3600A comprising a modification of the accessory 3600 shown in FIG. 70. Accessory 3600A includes a contact element 3620A having a wider and flatter configuration as compared to contact element 3620. The wider contact element 3620A includes a rib 3618A and a base 3622A which is larger in a widthwise direction as compared to the base 3622 of FIG. 70. The rib 3618A is less tapered as compared to rib 3618 and the surfaces 3619A intersecting at the top of the rib 3618A therefore define a larger angle relative to base 3622A as compared to the surfaces 3619 relative to base 3622 in FIG. 70. The increased width of contact element 3620A provides larger contact surfaces 3619A on either side of rib 3618A (as compared to contact surfaces 3619 in FIG. 70) which, in use, make contact with a patient-facing surface of the collapsible portion. As compared to the narrower contact element 3620 of FIG. 70, the wider contact element 3620A of FIG. 70A contacts and applies a reaction load across a larger area of the collapsible portion. The wider contact element 3620A may therefore apply a reaction load at a lower pressure as compared to the reaction load applied to the collapsible portion by the narrower contact element 3620.

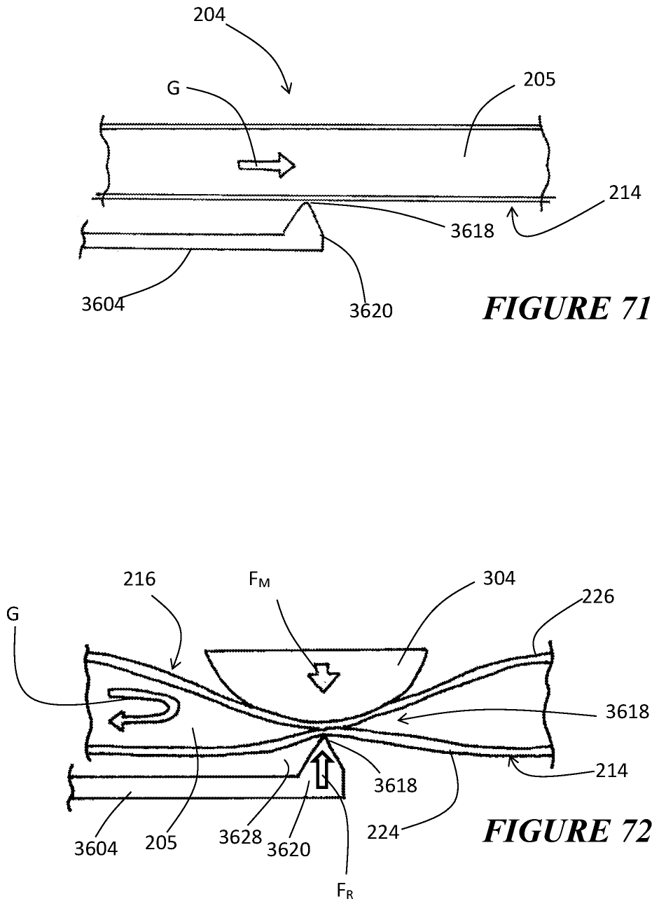

FIG. 71 illustrates the collapsible portion 204 overlying the contact element 3620 and whereby the patient-facing-surface 214 of the collapsible portion overlies and rests upon the tapered edge of rib 3618. The edge of the rib 3618 provides a relatively small area in contact with the patient-facing surface 214 such that the interface between the collapsible portion 204 and the contact element 3620 is relatively small. This configuration concentrates force onto a small area of the patient-facing-surface 3614 and thereby increases the pressure applied to the collapsible portion 204 by the accessory 3600.

FIG. 71 illustrates an uncollapsed configuration whereby lumen 205 of collapsible portion 204 is open and provides unobstructed passage for respiratory gases G. Turning to FIG. 72 there is illustrated a collapsed configuration where an inflatable mask cuff 304 is pressed against the non-patient-facing surface 216 of collapsible portion 204 at a position generally overlying the contact portion 3620. The applied force FM of the mask cuff 304 produces a reaction force FR applied to the patient-facing surface 214 by the contact element 3620 which collectively cause the non-patient side 226 and the patient-side 224 to collapse towards each other forming an obstruction 3618 in lumen 205 which restricts or occludes the respiratory gas flow G. It will be also be appreciated that the cross-sectional area of the lumen 205 is significantly reduced in the collapsed configuration illustrated in FIG. 72 as compared to the uncollapsed configuration illustrated in FIG. 71.

As shown in FIG. 72, the patient-facing side 224 of the collapsible portion 204 is partially collapsed or folded around the tapered rib 3618. A portion of the patient-facing side 224 is collapsed into a cavity 3628 between the contact element 3620 and the support arm 3604. Upon application of the force F_{M} from the mask cuff 304 onto the non-patient-side 226, the tapered edge of rib 3618 applies an equal and opposite reaction force F_{R} onto the patient-side 224. As shown in FIG. 72 the contact area between the mask cuff 304 and the non-patient-facing surface 216 is considerably larger than the contact area between the rib 3618 and the patient-facing surface 214. Consequentially, the reaction force F_{R} applied by the tapered rib 3618 is applies a higher pressure onto the patient-facing surface 214 than the pressure applied onto the non-patient-facing surface 216 by the applied mask force F_{M}. This configuration of accessory 3600 thereby amplifies the external pressure applied to the collapsible portion 204 in order to facilitate collapse of the collapsible portion. In some embodiments, the contact portion may be adjustable with respect to the clip such that, for example, the clip location may be adjusted with respect to the collapse location along the collapsible portion 204

As will be appreciated from the collapsed configurations illustrated in FIGS. 69 and 72, the contact portion of the accessory (exemplified in FIG. 69 by contact surface 3512 of accessory 3500 and in FIG. 72 by the contact element 3620 of the accessory 3600) are each in fixed relation to the clip 3502, 3602. The contact portion of the accessory facilitates collapse of the collapsible portion at a collapse location when the collapsing force of the mask cuff 304 is applied to the collapsible portion 204. The resulting collapse of the collapsible portion occurs at the location of the contact portion such that the collapse location may be said to be co-located with the collapse location. Furthermore, as the contact portion is in fixed relation to the clip, it will be appreciated that the collapse location is therefore also in fixed relation to the clip. Consequentially, adjustment of the clip location may allow for adjustment of the collapse location.

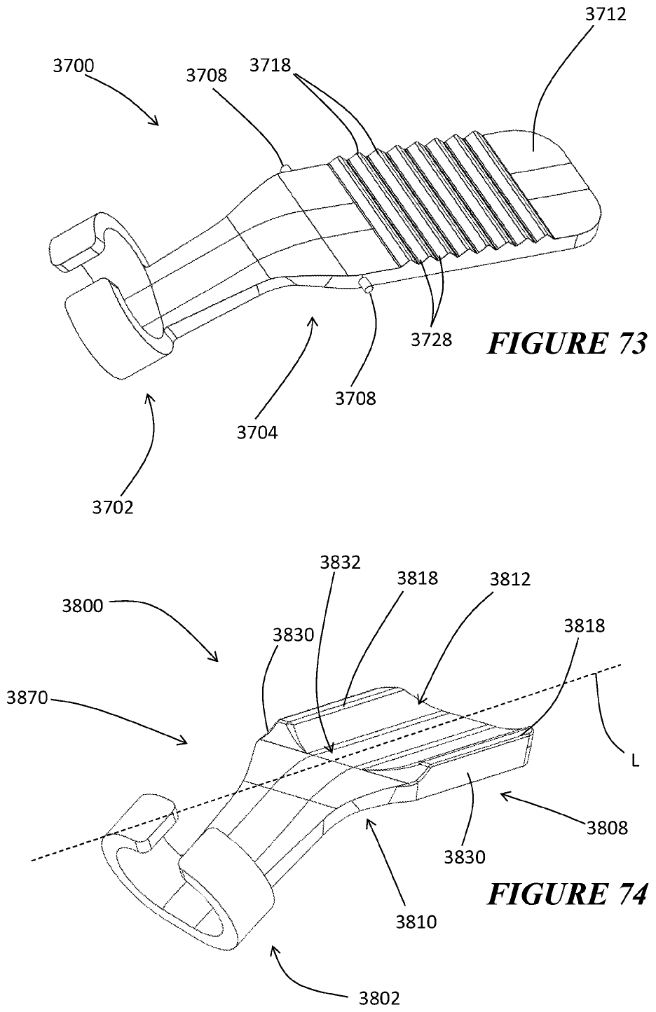

Turning to FIG. 73, there is illustrated an accessory 3700 according to another embodiment of the present disclosure. The accessory 3700 is similar in configuration to the aforementioned accessory 3500 illustrated in FIGS. 66 and 67 in that accessory 3700 includes a C-shaped clip 3702 and a curved backing plate 3704 extending from the clip 3702. Accessory 3700 differs from accessory 3500 in that the backing plate 3704 of accessory 3700 includes a plurality of ribs 3718 on its contact surface 3712. The plurality of ribs 3718 collectively define a serrated surface configured to promote and facilitate collapse of the collapsible portion 204.

The plurality of ribs 3618 are configured to concentrate force onto a plurality of discrete interfaces at the tip of each rib 3618. The valleys between adjacent ribs 3618 may also provide a series of cavities 3728 into which the patient-side 224 of the collapsible portion 204 may be collapsed into. This may assist in transforming lumen 205 into a tortuous or kinked passage of having flow resistance and thereby lowering or preventing residual flow through the collapsible portion 204 when in the collapsed configuration. The backing plate 3708 therefore provides a rigid support surface which contributes a reaction force onto the collapsible portion, as described above with reference to accessory 3500. Furthermore, the ribs 3718 may act to concentrate the reaction force and thereby amplify the pressure applied to the patient-facing surface of the collapsible portion. These combined effects thereby facilitate collapse of the collapsible portion.

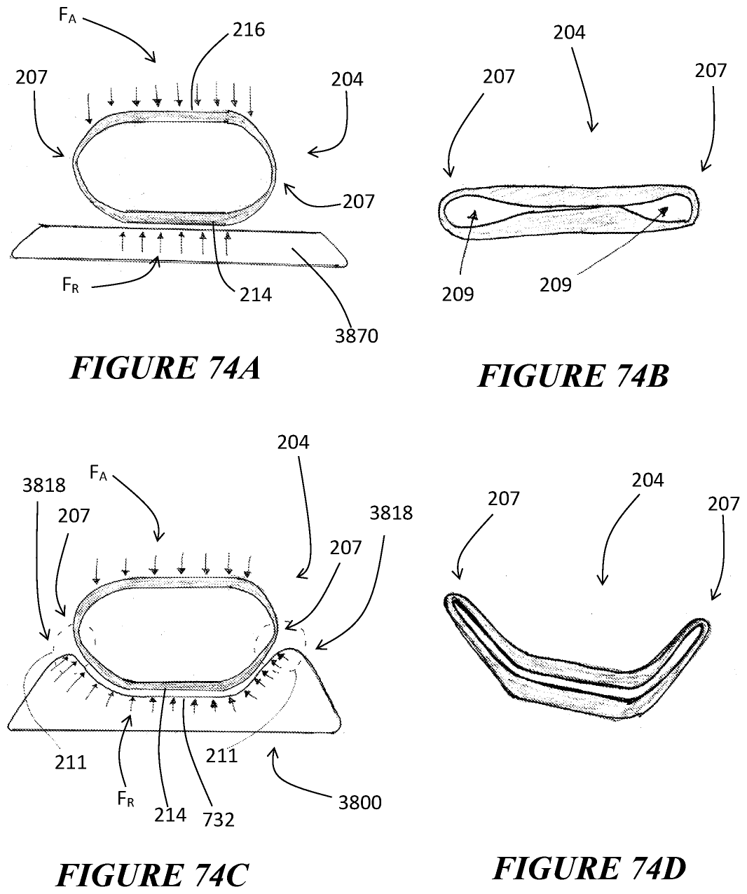

Turning to FIG. 74, there is illustrated an accessory 3800 according to another embodiment of the present disclosure. Similar to accessory 3500 and accessory 3700 of the preceding embodiments, accessory 3800 includes a C-shaped clip 3802 and a backing plate 3804 extends from the clip 3802 and includes a curved portion 3810 and a straight portion 3808 which has a longitudinal axis L. The straight portion 3808 includes a contact surface 3812 configured to contact the patient-facing surface of the collapsible portion. The contact surface 3812 includes a pair of longitudinal ribs 3818 orientated parallel with the longitudinal axis L and located adjacent a pair of opposite longitudinal sides 3830 of the straight portion 3808. A saddle-shaped seat 3832 is positioned between the ribs 3818. The seat 3832 is saddle-shaped when viewed in cross-section transverse to the longitudinal axis L, and it generally comprises a central base with opposing sides that extend at an angle away from the central base. The angle may be an obtuse angle. Each of the central base and opposing sides may be planar or curved. The seat 3832 may have a concave profile when viewed in a cross-section taken transverse to the longitudinal axis L (i.e. when viewed in the direction of longitudinal axis L). An embodiment of the saddle-shaped seat 3832 is illustrated in FIG. 74C.

In use, the ribs 3818 help locate the collapsible portion 204 onto the saddle 3832 such that the collapsible portion is restricted from movement in a direction perpendicular to axis L during application of pressure onto a non-patient side of the collapsible portion. The configuration of ribs 3818 and saddle 3832 therefore help maintain the collapsible potion in a desired position on contact surface 3812. Furthermore, the ribs 3818 promote a higher pressure applied to the edges of the collapsible portion along a longitudinal direction. This may help promote a more complete collapse of (and achieve greater occlusion of) the collapsible portion. In particular, this configuration may reduce or prevent the formation of longitudinally extending residual channels formed in one or more edge regions of the collapsible portion, when in the collapsed configuration. The longitudinal orientation of ribs 3818 and the positioning of ribs so as to engage opposing edge regions of the collapsible portion may increase pressure applied to the regions of the collapsible portion where said channels may be most likely to occur. Positioning ribs 3818 at these positions and in the longitudinal orientation may promote a higher pressure applied to the channels and thereby promoting sealing or narrowing of the channels or may prevent or reduce the formation of the channels.

The above-noted advantages of the accessory 3800 will be further described with reference to FIGS. 74A-74D.

FIG. 74A provides a cross-sectional view of the collapsible portion 204 overlying a flat (planar) backing plate 3870. An applied force FA is applied to a non-patient-facing surface 216 of the collapsible portion 204 and induces a reaction force F_{R} applied to a patient-facing-surface 214 by the backing plate 3870. It will be noted from the force arrows illustrated in FIG. 74A (and will otherwise be generally appreciated by the person skilled in the art) that the reaction force F_{R} is applied to the patient-facing-surface 214 only where contact occurs between the patient-facing surface 214 and the backing plate 3870. In some embodiments such as that which is illustrated in FIG. 74A, the reaction force F_{R} may generally not be applied at the side portions 207.

With reference to FIG. 74B, this may result in the formation of residual channels 209 within side portions 207 when the collapsible portion 204 is in the collapsed configuration. As shown in FIG. 74B, channels 209 are located at opposite edge regions of the collapsible portion 204 and extend longitudinally. The channels 209 provide a residual pathway for respiratory gases to pass through the collapsible portion 204 and contribute to residual gas flow present in the collapsed configuration. The design, dimensions and/or material of the collapsible portion can affect the formation and/or characteristics of such residual channels 209, for example a collapsible portion with a constant wall thickness could form larger residual channels 209 compared to a collapsible portion with a wall thickness that tapers towards an edge at each of the side portions 207. While a larger force F_{A} could be applied to the non-patient facing surface 216 to attempt to substantially reduce residual channels 209, such a force could cause damage and/or discomfort to the patient, and/or damage to the collapsible portion.

Turning to FIG. 74C, the collapsible portion 204 is shown in use with the accessory 3800 of FIG. 74. The patient-facing-surface 214 is seated within the saddle-shaped seat 3832 of accessory 3800. The side portions 207 of the collapsible portion 204 are contacted by the ribs 3818 such that the applied force F_{A} induces a reaction force F_{R} across a wider area of the patient-facing-surface 214, as compared to the arrangement in FIG. 74A. In particular, the reaction force F_{R} is imparted onto the side portions 207 at the interfaces 211 between the ribs 3818 and the side portions 207.

Turning to FIG. 74D, there is illustrated the collapsible portion 204 from FIG. 74C when in the collapsed configuration. As compared to FIG. 74B, it will be appreciated that the collapsible portion 204 of FIG. 74D has less or no instance of channel formation at the side portions 207. The longitudinal ribs 3818 have applied a reaction force F_{R} to the side portions 207 and therefore allowed for a more complete collapse at the side portions 207, as compared to the collapsed configuration shown in FIG. 74B. The collapsed configuration shown in FIG. 74D which was achieved by the accessory 3800 may therefore, in some instances, achieve lower levels of residual flow through the collapsible portion 204, as compared to the collapsed configuration shown in FIG. 74B which was achieved by the flat backing plate 3870.

The residual channels 209 may not necessarily form at edge regions of the collapsible portion 204 in the collapsed configuration, and could form anywhere along the width of the collapsed collapsible portion 204. Therefore, the accessory 3800 may be configured such that ribs 3818 may be located anywhere along the width of seat 3832 to facilitate collapse of such residual channels 209. In such instances, the seat 3832 may no longer be saddle-shaped and may comprise other shape profiles.

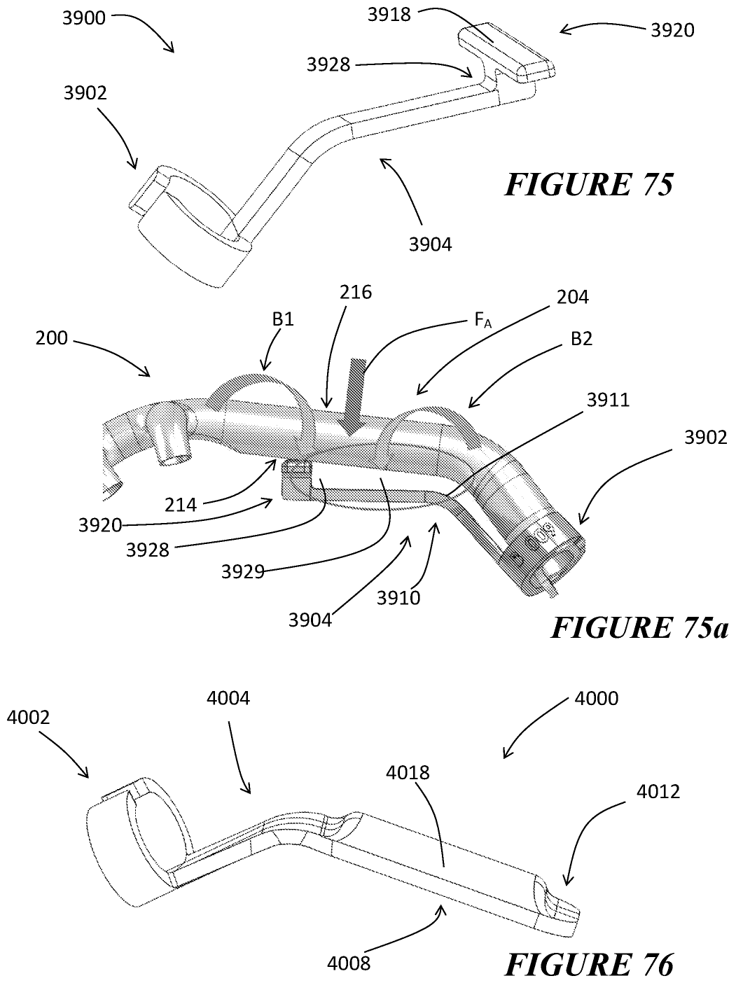

Turning to FIG. 75, there is illustrated an accessory 3900 according to another embodiment of the present disclosure. Accessory 3900 is similar in configuration to accessory 3600 illustrated in FIG. 70 insofar as accessory 3900 includes an attachment configuration comprising C-shaped clip 3902, a contact portion comprising contact element 3920 and a curved support member 3904 extending between the clip 3902 and the contact element 3920. In contract to the accessory 3600, the contact element 3920 includes a rectangular and flat abutment surface 3918 configured to contact the patient-facing surface of the collapsible portion. The abutment surface 3918 and the contact element 3920 each have a generally rectangular profile with a longitudinal axis that is generally perpendicular to a longitudinal axis of the support member 3904.

The abutment surface 3918 provides a rigid surface against which the non-patient side of the collapsible portion can be compressed by the applied external force (e.g. by a cuff of a patient mask or by a user's hand) and thereby facilitating collapse of the collapsible portion. The contact element 3920 extends in the non-patient facing direction from the support element such that a corner cavity 3928 is formed between the support member 3904 and the contact element 3920. In use, the corner cavity provides a volume for the patient-side of the collapsible portion to collapse into forming a tortuous or kinked flow path within the collapsible portion and thereby further reducing residual gas flow in the collapsed configuration.

FIG. 75a illustrates the accessory 3900 in use with a patient interface 200 and wherein a space 3929 is formed between a patient-facing surface 214 of the collapsible portion 204 and the support member 3904. The space 3929 includes the corner cavity 3928 as well as additional volume formed between the support member 3904 and the collapsible portion 204. The space 3929 is formed in a region upstream of the contact element 3920, with respect to a flow direction of respiratory gases in the gases delivery conduit. A section of the collapsible portion 204 spans (unsupported by the accessory 3900) between the contact element 3920 and the clip 3902. The collapsible portion 204 is thereby elevated or spaced from the patient's face.

An applied force F_{A} is applied to the non-patient-facing surface 216 of the collapsible portion 204. The contact element 3920 provides a pivot point about which the collapsible portion 204 may be folded upon application of the applied force F_{A}. The applied force F_{A} may induce a double bending moment in the collapsible portion. As indicated by arrows B1 and B2, the double bending comprising a first bending moment B1 occurring about the contact element 3920 and a second bending moment B2 occurring about the clip 3902 and/or the inside edge 3911 of the curved portion 3910 of the support member 3904. The collapsible portion may be caused to collapse, fold or kink in at least one position (and potentially two or more positions) and with the collapsible portion collapsing into the space 3929. As exemplified in FIG. 75a, the contact portion 3920 may be offset from the applied force F_{A}.

Still referring to FIG. 75a, whilst this particular embodiment illustrates the contact element 3920 contacting the patient-facing surface 214, it is noted that accessory 3900 could also be positioned such that the contact element does not contact the collapsible portion. For example, the contact element 3920 could contact a part of the gases delivery conduit downstream of the collapsible portion. In some embodiments, the contact element 3920 could contact a part of the gases delivery conduit upstream of the collapsible portion. In these instances, the collapsible portion may be entirely unsupported (at least directly) by accessory 3900. The unsupported collapsible portion 204 may span between supported sections of the gases delivery conduit. This arrangement may thereby facilitate collapse of the collapsible portion which, by virtue of it being unsupported, is allowed to fold or collapse into the space 3929.

Turning to FIG. 76, there is illustrated an accessory 4000 according to another embodiment of the present disclosure. The accessory 4000 has a configuration similar to the accessory 3500 in FIG. 66, the accessory 3700 in FIG. 73 and the accessory 700 in FIG. 74 insofar as accessory 4000 includes a C-shaped clip 4002 and a backing plate 4004 extending from the clip 4002. The backing plate 4004 has a pre-formed curve and a straight portion 4008. A non-patient-facing surface 4012 of the straight portion 4008 includes a contact portion comprising an elongate rib 4018 configured to contact the patient-facing surface of the collapsible portion. The rib 4018 has a longitudinal axis extending parallel with a longitudinal axis of the straight portion 4008. The rib 4018 is not tapered and may have a generally hump-shape (having a convex cross-sectional shape) comprising curved or contoured edges. In use, the patient-side of the collapsible portion is collapsed over the rib 4018. The crest of rib 4018 provides a rigid surface against which the non-patient side of the collapsible portion may be compressed under the force of an applied mask cuff.

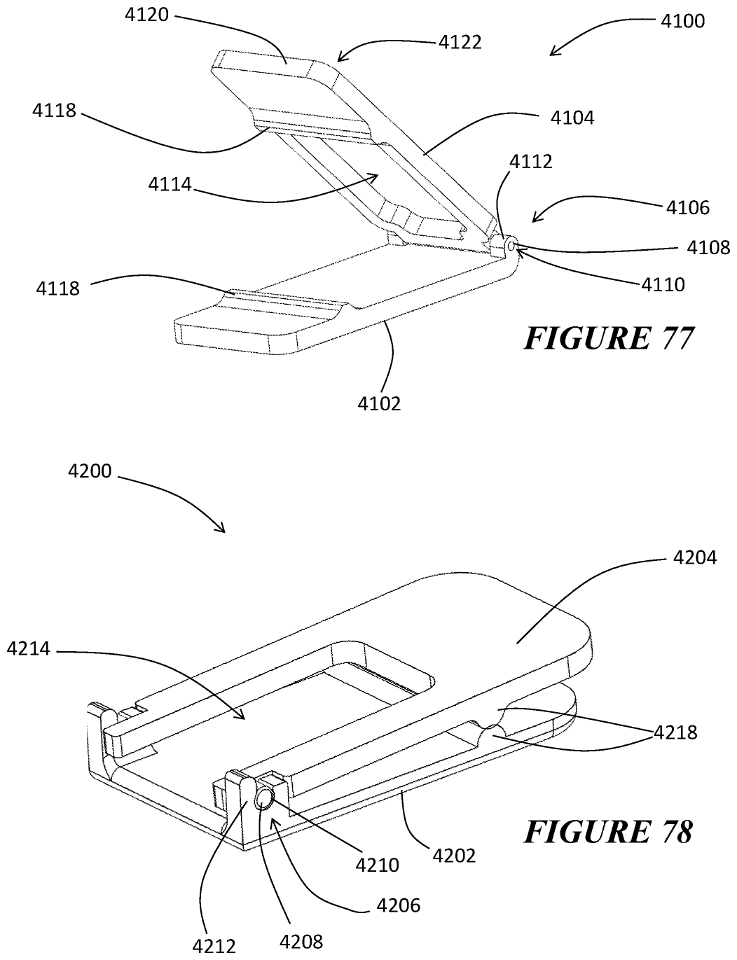

Turning to FIG. 77, there is illustrated an accessory 4100 according to another embodiment of the present disclosure. The accessory 4100 includes a base plate comprising a first lever arm 4102 locatable, in use, between the patient's face and a patient-facing surface of the collapsible portion. A second lever arm 4104 is hingedly connected to the first lever arm 4102 at a pair of identical hinge arrangements 4106 (only one of which is visible in FIG. 77). Each hinge arrangement 4106 comprises a hinge pin 4108 extending from the second lever arm 4104 and received in a corresponding opening 4110 formed in a hinge portion 4112 on the first lever arm 4102.

Each of the first and second lever arms 4102, 4104 include a contact portion comprising a rib 4118 for concentrating force onto the collapsible portion. The second lever arm 4104 includes an opening 4114 configured to receive the collapsible portion and whereby, in use, the collapsible portion extends through the opening and is located between the first and second lever arms 4102, 4104. In an alternative embodiment, the first level arm 4102 includes the opening 4114 configured to receive the collapsible portion and whereby, in use, the collapsible portion extends through the opening and is located between the first and second lever arms 4102, 4104.

A distal end of the second lever arm 4104 provides a force-application portion 4120 for receiving the applied force from a bag mask applied to the patient's face. The force-application portion includes an application surface 4122 onto which the force is applied and whereupon the second lever arm 4104 is rotated about the hinge arrangements 4106 toward the first lever arm 4102 causing the collapsible portion to become clamped and squeezed between the ribs 4118 of the first and second lever arms 4102, 4104.

The rib 4118 of the second lever arm 4104 is positioned between the application surface 4122 and the hinge arrangements 4106 such that the accessory 4100 comprises a second-class lever configuration i.e. a ‘nut-cracker’ lever configuration. The force delivered to the collapsible portion at the ribs 4118 is therefore an amplification of the force applied to the application surface 4122. The extent of the force amplification will be dependent on the particular configuration of the lever arms and, in particular, the distance between the application surface 4122 and the rib 4118 on the second lever arm 4104. It will be appreciated that the greater the distance between the rib 4118 on the second lever arm 4104 and the application surface 4122, the greater the degree of force amplification. For example, the degree of force amplification may be increased by either positioning rib 4118 closer to hinge arrangement 4106 and/or by increasing the length of the second lever arm 4104 such that the distance between application surface 4122 and the rib 4118 is increased.

Turning to FIG. 78, there is illustrated a modification of the embodiment illustrated in FIG. 77. FIG. 78 illustrates an accessory 4200 having a similar configuration to the accessory 4100 of FIG. 77 except that the opening 4214 of accessory 4200 is an open-sided opening as opposed to the enclosed opening 1014 of accessory 4100. The open-sided opening 4214 is open-ended at proximal end of the second lever arm 4204 and may therefore allow the collapsible portion to extend through the opening 4214 with less obstruction when in the uncollapsed configuration. Furthermore, the open-ended opening 4214 of accessory 4200 enables the second lever arm to be fitted onto the collapsible portion without requiring disconnection of the patient interface to feed the collapsible portion through the opening.

The accessory 4200 also differs from the accessory 4100 in that the hinge arrangement 4206 of accessory 4200 has an open configuration whereby hinge pins 4208 are snap-fitted into a pin recess 4210 formed in hinge portions 4212. This configuration allows for the second lever arm 4204 to be disconnected from the first lever arm 4202 and subsequently reconnected by snap-fitting the hinge pins 4208 into the recesses 4200. In use, the accessory 4200 may be installed onto the gas delivery conduit during operation of the patient interface by temporarily disconnecting the first and second lever arms 4202, 4204 from each other such that the first lever arm 4202 is located between the patient's face and a patient-facing surface of the collapsible portion. The second lever arm 4204 may then be fitted over the gases delivery conduit which is received within the open-ended opening 4214 and the second lever arm 4204 then snap-fitted together with the first lever arm 4202 at the hinge arrangements 4206. The accessory 4200 may be connected onto an operational patient interface and then slid along the gases delivery conduit until the collapsible portion is located between the ribs 4218. This configuration may also allow for the accessory to be conveniently removed from an operational patient interface, if required.

It will be appreciated that the accessory 4100 in FIG. 77 and accessory 4200 in FIG. 78 provide a lever configuration which can amplify the force of the applied load when it is applied to a position further from the hinge than the ribs 4218. There may be alternative instances of use (or alternative configurations of the accessory) where the force is applied to the second lever arm at the same distance from the hinge as the ribs. In such an instance, there may be no amplification of the force applied. However, the accessory will nonetheless still function as a clamping mechanism with delivers force to both sides of the collapsible portion (and concentrated at the ribs). The accessory will also still deliver the applied force via the ribs 4118 which concentrate (and thereby increase pressure) of the applied force. According to this configuration, the accessory still facilitates collapse of the collapsible portion.

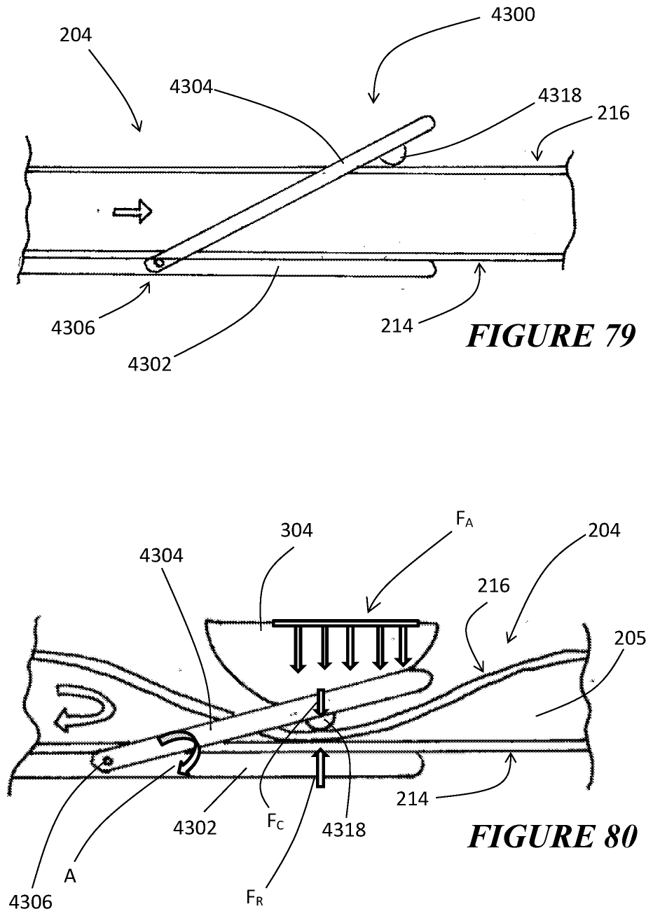

Turning to FIGS. 79 and 80, there is illustrated another embodiment of the present disclosure. An accessory 4300 includes a base plate 4302 and a pivot arm 4304 pivotally connected to the base plate 4302 at a pivot 4306. The collapsible portion is located between the base plate 4302 and pivot arm 4304 with the collapsible portion 204 extending through an opening in pivot arm 4304 which is not visible in FIGS. 79 and 80 but may be similar or equivalent to the openings 4114 and 4214 illustrated in FIGS. 77 and 78.

As shown in FIG. 79, a patient-facing surface 214 of the collapsible portion 204 overlies and is in contact with the base plate 4302. The rib 4318 rests upon the non-patient-facing surface 216 of the collapsible portion 204. The resiliency of the collapsible portion is sufficient to support and maintain the pivot arm 4304 in the open configuration shown in FIG. 79.

Turning to FIG. 80, application of an applied force F_{A} from a bag mask cuff 304 onto the pivot arm 4304 is sufficient to overcome the resiliency of the collapsible portion 204 and cause the pivot arm to rotate toward the base plate 4302 in the clockwise direction indicated by arrow A. The rib 4318 concentrates the applied force F_{A} of the mask cuff 304 onto a relatively small area on the non-patient-facing surface 216 and thereby applying a higher pressure to the collapsible portion than was applied to the pivot arm 4304 by the mask cuff 304. In particular, the applied force F_{A} which is spread along a section of the pivot arm 4304 is concentrated at the rib 4318 into a collapsing force F_{C} applied to the collapsible portion 204. The collapsing force Fc produces an equivalent reaction force F_{R} applied to the patient-facing-surface 214 by the base plate 4302. As shown in FIG. 80, the collapsible portion is clamped between the rib 4318 and the base plate 4302 forming an obstruction within the collapsible portion and occluding respiratory gases pathway within the lumen 205.

As shown in FIG. 80, the applied force F_{A} is applied along a distal end of the pivot arm 4304. The applied force F_{A} may be a UDL (uniformly distributed load) or a UVL load (uniformly varying load) or may be a point load. The applied force F_{A} is applied, on average, at a position further from the pivot 4306 than the rib 4318 is to the pivot 4306. The collapsing force F_{C} applied by the rib is therefore an amplification of the applied force F_{A}.

In some embodiments, the base plate 4302 may be provided by the backing plate of one of the above-discussed embodiments of the disclosure. Returning briefly to FIG. 73, a pair of hinge pins 3708 extend from side edges of the backing plate 3704. The hinge pins 3708 allow for a pivot arm such as the pivot arm 4304 described with reference to FIG. 79 to be connected to backing plate 3704 shown in FIG. 73.

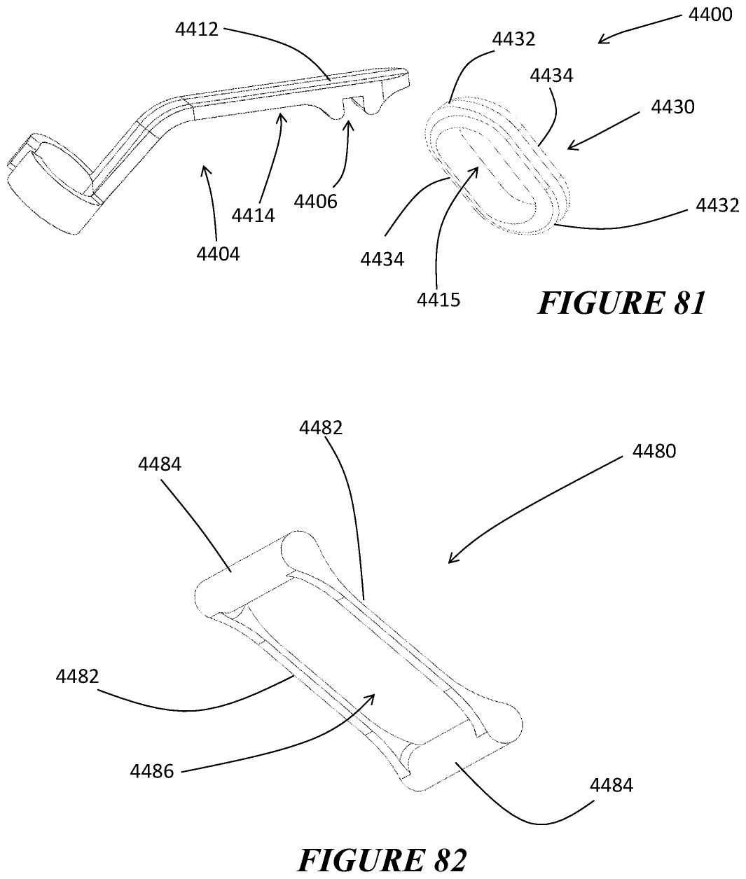

An earlier embodiment of an accessory intended to facilitate collapse of a collapsible portion was disclosed in Applicant's International Patent Application PCT/IB2019/051137. FIGS. 25A-F of that disclosure illustrated a ring 215 configured to extend around the conduit and to tilt or rotate when a force is applied to the ring 215 in order to pinch or kink the conduit. This concept has been refined and advantageous improvements developed which are illustrated in FIGS. 81-84. Turning to FIGS. 81-84, there is illustrated an accessory 4400 according to a further embodiment of the present disclosure. The accessory 4400 comprises a backing plate and pivot member assembly. In particular, accessory 4400 includes a backing plate 4404 similar to the accessory 3500 illustrated in FIG. 66 but also including a pivot member recess 4406 located on a patient-facing side 4414 of the backing plate 4404. The pivot member recess 4406 is configured to receive and engage with a pivot member and to permit pivotal movement of the pivot member relative to the backing plate 4404. The pivot member may be engageable with pivot member recess 4406 via a snap-fit connection. The pivot member may be engageable with pivot member recess 4406 via a one-time or removable connection.

In the embodiment illustrated in FIG. 81, the pivot member comprises a pivot ring 4430 configured to seat within the ring recess 4406. The pivot ring 4430 includes a central opening 4415 configured for the collapsible portion to extend therethrough. The pivot ring 4430 includes a pair of semi-circular sections 4432 connected by a pair of straight sections 4434.

FIG. 82 includes an alternative pivot member comprising a pivot arm 4480 which could also be used with backing plate 4404 in the accessory 4400 and instead of pivot ring 4430. The pivot arm 4480 comprises a pair of side members 4482 extending between a pair of cylindrical bars 4484. The bars 4484 are spaced apart from one another by the length of the side members 4482. The pivot arm 4480 includes a central opening 4486.

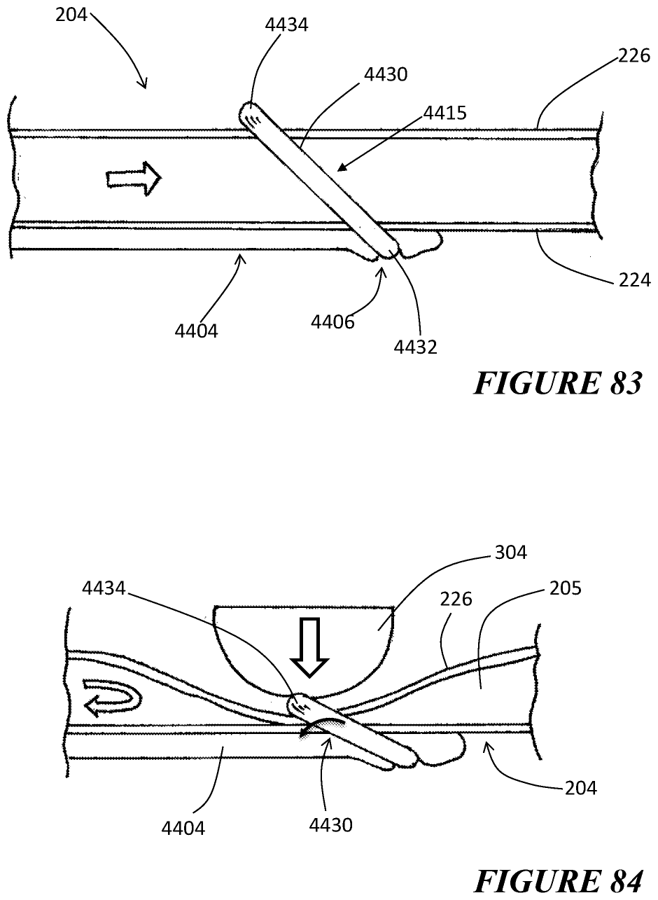

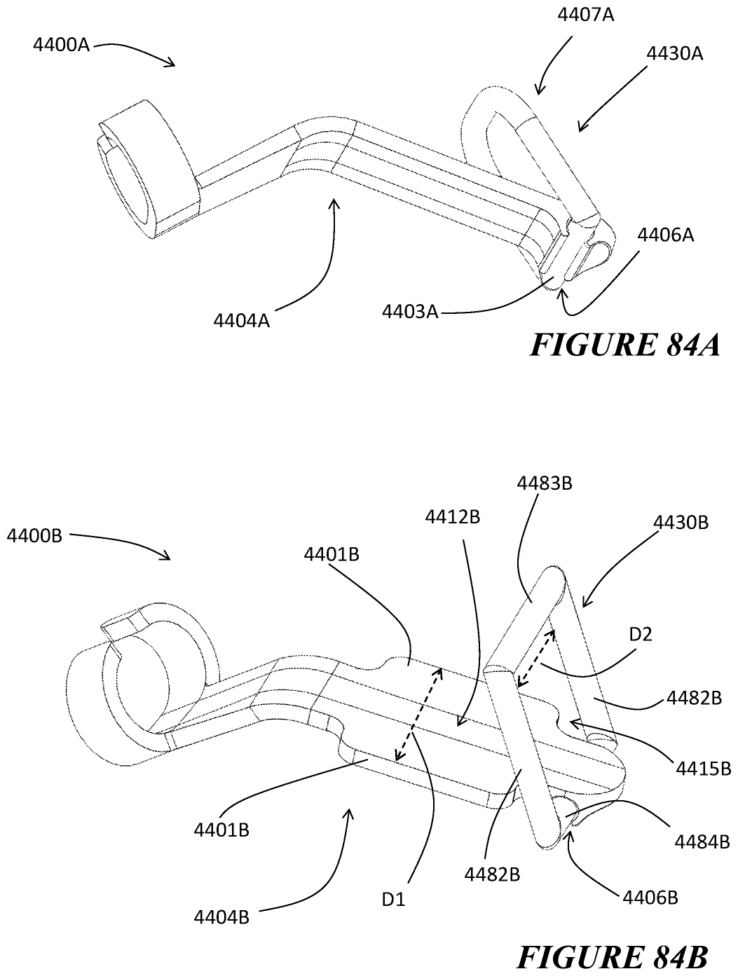

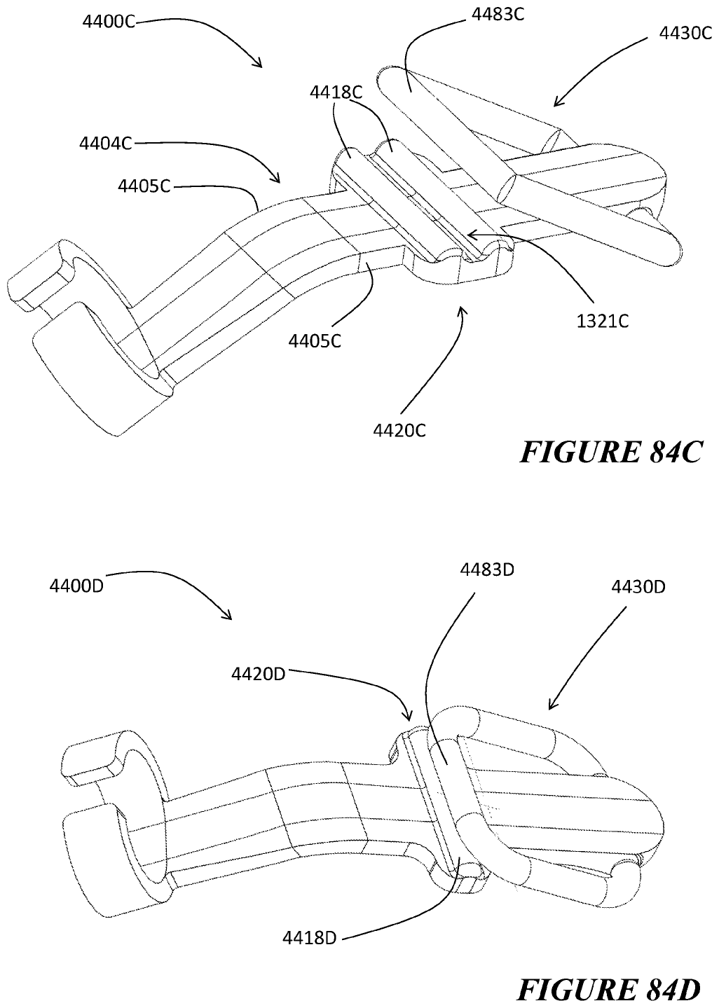

FIGS. 83 and 84 illustrate operation of the assembly illustrated in FIG. 81 comprising the backing plate 4404 and pivot ring 4430. As illustrated in FIG. 83, the collapsible portion 204 extends through the opening 4415 and a patient-side 224 of the collapsible portion 204 overlies the backing plate 4404. A patient-side 4432 of the pivot ring 4430 is received and pivotally engaged with the recess 4406 so as to permit pivoting movement of the pivot ring 4430 with respect to the backing plate 4404. FIG. 83 illustrates an uncollapsed configuration wherein a non-patient-side 4434 of the pivot ring 4430 is resting on the non-patient side 226 of the collapsible portion.

FIG. 84 illustrates a collapsed configuration wherein a mask cuff 304 is pressed down onto the non-patient-side 4434 of the pivot ring 4430 causing anticlockwise (as seen from the perspective in FIGS. 82 and 84) rotation of the pivot ring 4430 toward the backing plate 4404 and causing the collapse of the non-patient-side 226 of the collapsible portion 204. The collapsible portion 204 is clamped and pinched between the pivot ring 4430 and the backing plate 4404 forming an obstruction within the lumen 205 of the collapsible portion 204.

Whilst FIGS. 83 and 84 illustrate operation of the accessory 4400 using an assembly of the backing plate 4404 and pivot ring 4430, it will be appreciated that the accessory 4400 could also comprise an assembly of backing plate 4404 and pivot arm 4480. In this configuration, a first of the bars 4484 is received and engaged with the recess 4406 so as to permit pivotal movement of the pivot arm 4480 relative to the backing plate 4404. The collapsible portion 204 extends through the central opening 4486 of the pivot arm 4480 and the second of the bars 4484 rests on the non-patient-facing side 226 of the collapsible portion 204.

In an alternative embodiment (not illustrated) a variation of the pivot arm 4480 includes only a single side member 4482 such that the pair of cylindrical bars 4484 and a single side member 4482 form a U-shape.

The combination of a backing plate 4404 and pivot ring 4430 provides a significant improvement over the ring 215 disclosed in Applicant's earlier patent application PCT/IB2019/051137. In particular, the backing plate 4403 enables the ring 4430 to be properly located at a desired position. Furthermore, the recess 4406 provides improved rotational movement of the pivot ring 4430 which further enhances the collapse achieved during use.

Turning to FIGS. 84A-84D, there are illustrated four alternative embodiments of an accessory according to the present disclosure. These embodiments are modifications of the operational concept provided by accessory 4400 and illustrated in FIGS. 81, 83 and 84.

FIG. 84A illustrates an accessory 4400A in which the backing plate 4404A is equivalent to backing plate 4404 of accessory 4400 but in which the pivot ring comprises a D-shaped ring 4430A, as opposed to the oval-shaped pivot ring 4430 illustrated in FIG. 81. The D-shaped ring 4430A comprises a straight portion 4403A and a U-shaped portion 4407A extending from the straight portion and which collectively form a D-shape. The straight portion 4403A is received within the pivot member recess 4406A and, in use, rotates within the recess 4406A to permit pivoting movement of the D-shaped ring 4430A relative to the backing plate 4404A.

FIG. 84B illustrates an accessory 4400B comprising a backing plate 4404B and a pivot member 4430B. The pivot member 4430B has square configuration formed of four cylindrical members comprising a pair of side members 4482B extending between a top member 4483B and a bottom member 4484B which is located in the pivot member recess 4406B on the underside of the backing plate 4404B. The pivot member 4430B includes a central opening 4415B. In use, a collapsible portion of a gases delivery conduit extends through the opening 4415B.

The backing plate 4404B is equivalent in configuration to the backing plate 4404 illustrated in FIGS. 81 and 84 with the exception that the backing plate 4404B includes side projections 4401B which provide an enlarged contact surface 4412B as compared to the contact surface 4412 of the backing plate 4404 which is illustrated in FIG. 81. The side projections 4401B may further facilitate collapse of the collapsible portion by ensuring that the longitudinal side portions of the collapsible portion are properly supported and do not become folded over a side edge of the backing plate 4404B.

Furthermore, a distance D1 between the outer edges of the side projections 4401B is larger than a distance D2 between the inner edges of the side members 4482B. When accessory 4400B is not attached to a patient interface, pivoting movement of the pivot arm 4430B toward the contact surface 4412B will therefore result in contact between the side members 4482B and the side projections 4401B. In use, when accessory 4400B is attached to a patient interface and wherein the collapsible portion of a gases delivery conduit extends through the opening 4415B, pivoting movement of the pivot arm 4430B toward the contact surface 4412B causes the collapsible portion to become pinched between the pivot member 4430B and the contact surface 4412B. In particular, the longitudinal side portions (for example side portions 207 illustrated in FIGS. 84A-84D) of the collapsible portion may become pinched between the side projections 4401B and the side members 4482B. The accessory 4400B may thereby minimise the formation of longitudinal channels in longitudinal side portions of the collapsible portion and, in turn, further reducing residual flow through the collapsible portion when in the collapsed configuration.

Turning to FIG. 84C, there is illustrated an accessory 4400C according to another embodiment of the present disclosure. The accessory 4400C is similar in configuration to the accessory 4400A with the exception that the accessory 4400C includes a modified backing plate 4404C which comprises a contact portion 4420C. The contact portion 4420C includes a pair of elongate ribs 4418C orientated perpendicularly to a longitudinal axis of the backing plate 4404C. The ribs 4418C are longer in their lengthwise direction than the width of the backing plate 4404C. The rib 4418C therefore extend beyond the longitudinal edges 4405C of the backing plate 4404C. The pair of ribs 4418C are positioned with respect to the pivot arm 4430C such that the top member 4483C of the pivot member 4430C is seatable in a recess 4421C between the ribs 4418C, when the pivot member is pivoted toward the backing plate 4404C. The recess 4421C is therefore positioned within the arcuate path of the top member 4483C. In use, a part of the collapsible portion is pinched between the recess 4421C and the top member 4483C.

This configuration may urge part of the collapsible portion to kink or fold into the recess 4421C. This configuration may also provide two pinch points in that the collapsible portion is pinched between, firstly, the top member 4483C and a first of the ribs 4418C and, secondly, the top member 4483C and a second of the ribs 4418C. The contact portion 4420C is configured to contact a patient-facing-surface of the collapsible portion at a collapse location and to thereby facilitate collapse of the collapsible portion when the collapsible portion is pinched between ribs 4418C and a top member 4483C of the pivot member 4430C.

Turning to FIG. 84D, there is illustrated an accessory 4400D according to another embodiment of the present disclosure. The accessory 4400D is similar in configuration to accessory 4400C of FIG. 84C except that the contact portion 4420D comprises a single rib 4418D instead of the double-rib configuration of contact portion 4420C in FIG. 84C. The single rib 4418D is positioned in the arcuate path of the top member 4483D of the pivot arm 4430D. Movement of the pivot arm toward the contact portion 4420D will result in contact between the top member 4483D and the top of the rib 4418D. Consequentially, in use, the collapsible portion will become pinched between the top member 4483D and the rib 4418D. This pinch point thereby defines a collapse location at which the accessory 4400D can, in use, induce collapse of the collapsible portion.

As with accessory 4400 illustrated in FIGS. 81, 83 and 84, it will be appreciated that for each accessory illustrated in FIGS. 84A-84D, the gases delivery conduit of a patent interface extends through the central opening in the pivot member 4430A-D and overlies the backing plate 4403A-D. Application of an external force, for example from a bag mask being applied to the patient's face, contacting the top member 4483A-D causes the pivot member 4430A-D to rotate within the pivot arm recess and for the collapsible portion to become pinched between the top member 4483A-D and the backing plate 4404A-D, and thereby facilitating collapse of the collapsible portion.

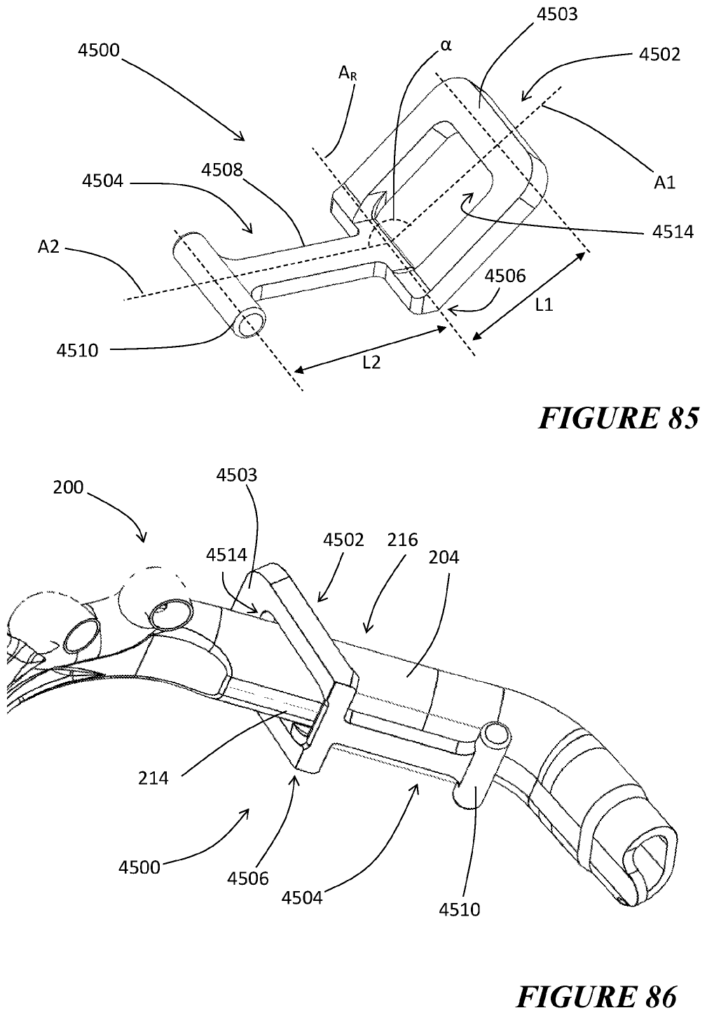

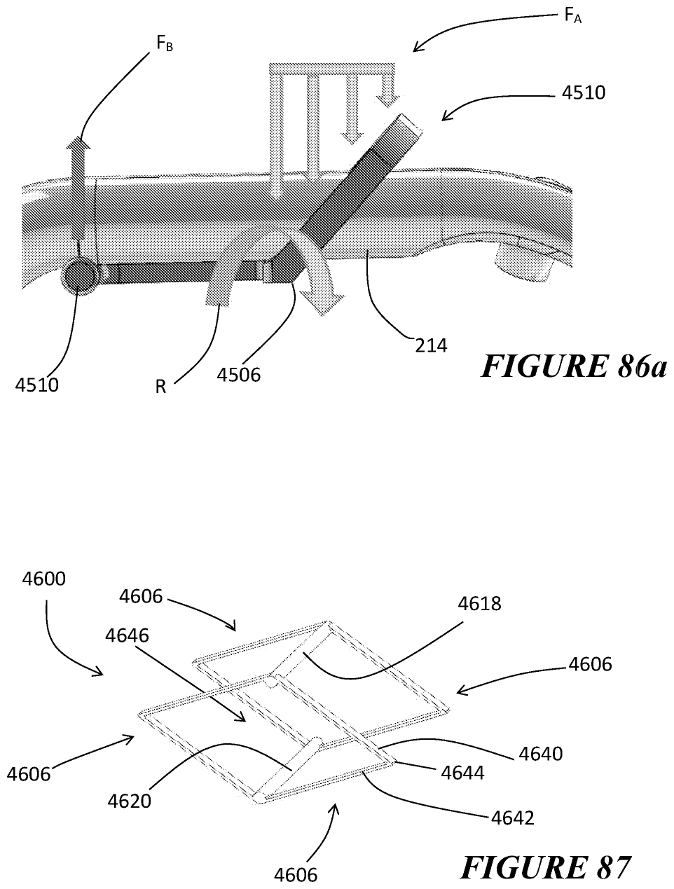

Turning to FIGS. 85 and 86, there is illustrated an accessory 4500 according to another embodiment of the present disclosure. The accessory 4500 comprises a rigid and one-part component having a ‘see-saw’ configuration wherein a first lever arm 4502 and a second lever arm 4504 extend on either side of a fulcrum 4506. The first lever arm 4502 and second lever arm 4504 are angled with respect to each other so as to generally form a V-shape.

The first lever arm 4502 comprises a C-shaped member extending outwardly from the fulcrum 4506. The first lever arm 4502 defines an opening 4514 which provides an attachment configuration allowing attachment of the accessory to the collapsible portion. In particular, the opening 4514 is configured for the collapsible portion 204 to, in use, extend therethrough.

The second lever arm 4504 comprises an elongate bar 4508 extending from the fulcrum 4506 and a cylindrical bar 4510 at the distal end of elongate bar 4508. The cylindrical bar 4510 has a longitudinal axis perpendicular to a longitudinal axis of the elongate bar 4508. The angle α between the longitudinal axes of the first and second lever arms 4502, 4504 may depend on the length or other configuration of the first and second lever arms. According to a particular embodiment the angle α is an obtuse angle i.e. between 90-180°.

The first lever arm 4502 has a longitudinal axis A1. The second lever arm 4504 has a longitudinal axis A1. The fulcrum comprises a rigid corner 4506 extending along an interface of the first and second lever arms 4502, 4504 and perpendicularly to the longitudinal axes A1, A2. The corner 4506 provides a tilt edge and defines an axis of rotation AR about which the first and second lever arms 4502, 4504 can rotate when the corner 4506 is located on a surface.

The first lever arm 4502 includes a contact section 4503 extending generally parallel with the corner 4506. The contact section 4503 and the cylindrical bar 4510 provide a pair of distinct contact portions configured to apply a collapsing force to the collapsible portion, as will be discussed in further detail below with reference to FIG. 86. The contact section 4503 and the cylindrical bar 4510 may be spaced unequally from the corner 4506. As shown in FIG. 85, the cylindrical bar 4510 may be spaced from the axis of rotation AR by a distance L2 and the contact section 4503 may be spaced from the axis of rotation AR by a distance L1. According to a particular embodiment, the distance L1 is less than the distance L2. That is, the contact section 4503 is spaced closer the axis of rotation AR than the cylindrical bar 4510 is spaced to the axis of rotation AR.

FIG. 86 illustrates the accessory 4500 fitted to patient interface 200. The collapsible portion 204 extends through the opening 4514 of the first lever arm 4502. The second lever arm 4504 is in contact with a patient-facing surface 214 of the collapsible portion 204 and, in use, is located between the patient's face and the patient-facing surface 214. FIG. 86 illustrates an uncollapsed configuration in which the accessory 4500 is fitted to the collapsible portion 204 but where no external force is being applied to the accessory. The second lever arm 4504 is therefore at rest on a support surface (not shown) such as the patient's face or on an optional backing plate. The contact portion 4503 of the first lever arm 4502 is at rest upon the non-patient-facing surface 216 of the collapsible portion 204.

In use, the application of a force such as a force from a bag mask is applied to the contact portion 4503 of the first lever arm 4502 and in a direction toward the patient's face. The applied force causes the accessory 4500 to pivot about the corner 4506 and causing movement of the second lever arm 4504 in a direction away from the patient's face and toward the collapsible portion 204. The movement of the second lever arm 4504 causes the cylindrical bar 4510 to press into the patient-facing surface 214 of the collapsible portion 204. Simultaneously, the pivoting movement of the accessory 4500 causes the contact portion 4503 of the first lever arm 4502 to press into the non-patient-facing surface 216 of the collapsible portion 204. The collapsible portion 204 is therefore caused to collapse at least at two separate and spaced apart locations and in two different directions by the contact portion 4503 and cylindrical bar 4510 respectively.

FIG. 86a provides a side view of the arrangement of FIG. 86 and in which the rotational movement and relevant forces are indicated. The applied force F_{A} is applied to the first lever arm 4502 causing rotation R in the clockwise direction about the corner 4506 such that the cylindrical bar 4510 delivers a force F_{B} to the patient-facing surface 214. In this manner, the applied force F_{A} is translated to the bar 4510 on the opposite side of the collapsible portion 204, promoting a forced collapse at the position of the bar 4510 in addition to the collapse occurring beneath the first lever arm 4502 as a result of the applied force F_{A}.

Turning to FIG. 87, there is illustrated an accessory 4600 according to another embodiment of the present disclosure. The accessory 4600 includes a pair of contact portions comprising a first cylindrical bar 4618 and second cylindrical bar 4620. In the perspective shown in FIG. 87, the first cylindrical bar is orientated above the second cylindrical bar and so the first cylindrical bar will hereinafter be referred to as the upper bar 4618 and the second cylindrical bar will be referred to as the lower bar 4620.

The upper and lower bars 4618, 4620 are connected to one another in spaced apart relation by a biasing arrangement comprising four flexibly resilient linkages 4606. The linkages 4606 have a normally non-linear formation and, in particular, have a V-shaped configuration. In alternative embodiments the linkages may have a C-shaped or U-shaped configuration. The linkages 4606 each comprise an upper portion 4640 and a lower portion 4642 connected at a corner 4640. The upper portions 4640 extend between the upper bar 4618 and the corner 4640. The lower portions 4642 extend between the corner 4640 and the lower bar 4620. As shown in FIG. 87, the upper portions 4640 extend from opposite sides of the upper bar 4618 and the lower portions extend from opposite sides of the lower bar 4620. The linkages 4606 connect to the upper and lower bars 4618, 4620 adjacent opposite ends of the upper and lower bars 4618, 4620. As seen from FIG. 87, each pair of V-shaped linkages 4606 at each end of the upper and lower bars 4618, 4620 form a diamond-shaped configuration.

The linkages 4606 have a normal configuration shown in FIG. 87 such that the upper and lowers bars 4618, 4620 will be normally spaced apart by a distance approximately corresponding to the thickness of the collapsible portion 204. The resiliency of the linkages 4606 urge the upper and lowers bars 4618, 4620 to return to the spacing of the normal configuration when an applied external force moves the upper and lowers bars 4618, 4620 to an alternative spacing. The spacing between the upper and lower bars 4618, 4620 defines an opening 4646 through which the collapsible portion 204 is, in use, inserted. The opening 4646 is bounded by the upper and lower bars 4618, 4620 and by the linkages 4606 so as to define an enclosed opening. As will be appreciated from FIG. 87, the accessory is symmetrical across X, Y and Z planes.

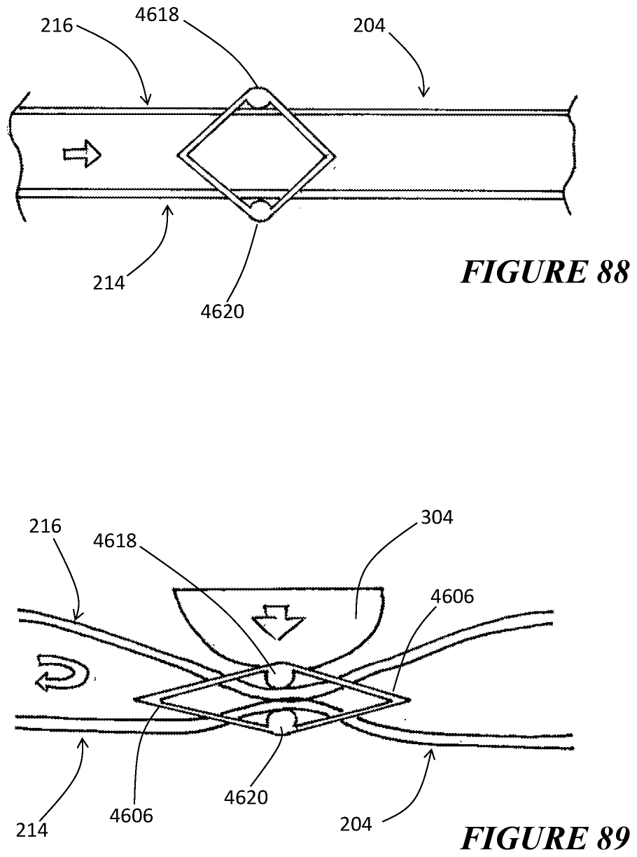

Turning to FIG. 88, the accessory 4600 is attached to the collapsible portion 204 by virtue of the collapsible portion 204 extending through opening 4646. The upper bar 4618 overlies and is in contact with a non-patient-facing surface 216 of the collapsible portion 204. The lower bar 4620 underlies and is in contact with a patient-facing surface 214 of the collapsible portion 204. FIG. 88 illustrates an uncollapsed configuration wherein the collapsible portion 204 is open and unobstructed and wherein the accessory 4600 is in its normal (i.e. at rest) configuration.

FIG. 89 illustrates an in-use view of the accessory 4600 wherein an applied force from a patient mask cuff 304 is pressed onto the upper bar 4618 causing linkages 4606 to resiliently deform and the upper bar 4618 to move toward the lower bar 4620. The section of collapsible portion which extends through opening 4646 is collapsed and pinched between the upper and lower bars 4618, 4620. The upper and lower bars 4618, 4620 may therefore act as clamping members to facilitate collapse of the collapsible portion. The upper and lower bars 4618, 4620 are, in use, forced together creating two points of localised pressure on the surface of the collapsible portion 204 at the non-patient-facing surface 216 and the patient-facing surface 214. The upper and lower bars 4618 4620 may be aligned along the same plane (as illustrated in FIG. 89). Alternatively, the bars could be offset to one another thereby creating a tortuous path in the collapsible portion when the pinch occurs. The portion of the bars that contact the collapsible portion could be configured with an edge (for example, a tapered edge) to concentrate load and thereby amplifying pressure.

The diameter of the upper and lowers bars 4618 and 4620 is relatively small and, in particular, is smaller than the width of the mask cuff 304. The force from the mask cuff 304 is thereby concentrated onto the collapsible portion 204 at the upper and lowers bars 4618 and 4620 which consequentially delivers a pressure onto the collapsible portion 204 which is greater than the pressure applied by the mask cuff 304. As shown in FIG. 89, in the collapsed configuration the patient-facing-surface 214 may become partially wrapped or curved around the lower bar 4620 and the non-patient facing surface 216 may become partially wrapped or curved around the upper bar 4618.

Upon release of the load applied by the mask cuff 304, the resiliency of the linkages 4606 will urge the accessory 4600 to return to the normal configuration shown in FIGS. 87 and 28. The resiliency of the collapsible portion 204 and/or the pressure of the respiratory airflow provided through the collapsible portion 204 may in part cause the collapsible portion 204 to return to the uncollapsed configuration illustrated in FIG. 88. According to an alternative (not illustrated) embodiment of the accessory 4600, the accessory may include a clip and arm configuration which attaches on top of the patient interface between the collapsible portion and the gas path connector in order to locate the accessory at the appropriate location.

The resiliency of linkages 4606 may be due to material and/or geometry. The accessory 4600 may be formed of more than one material and whereby different parts of the accessory are formed of material which contributes to the desired characteristics of that part. For example, the linkages 4606 may be formed of a resilient material to enable their flexibly resilient function whereas the upper and lower bars 4618, 4620 may be formed of a rigid material.

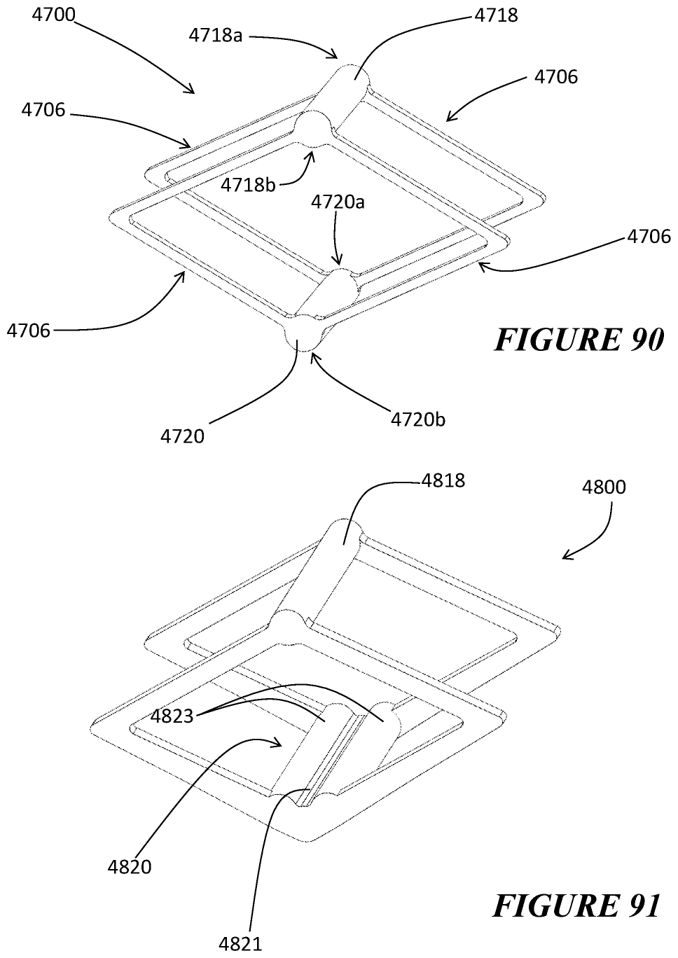

FIGS. 90 and 91 illustrate modifications of the accessory 4600 illustrated in FIGS. 87-89. FIG. 90 illustrates an accessory 4700 which is similar in configuration to accessory 4600 having in that it comprises an upper bar 4718 and a lower bar 4720 connected and spaced apart from one another by four resilient flexible v-shaped linkages 4706. Accessory 4700 differs from accessory 4600 in that, in accessory 4600, the flexible linkages connect to an upper part of the upper bar 4618 and to a lower part of the lower bar 4620. In contrast to accessory 4600, and as shown in FIG. 90, the linkages 4706 of accessory 4700 connect to a lower part 4718b of the upper bar 4718 and to an upper part 4720a of the lower bar 4720. The connections between the linkages 4706 and the upper and lower bars 4718, 4720 are therefore positioned further inward as compared to that of accessory 4600. Consequentially, the upper part 4718a of the upper bar 4718 and the lower part 4720b of the lower bar 4720 protrude farther in the upper and lower directions as compared to accessory 4600.

FIG. 91 illustrates an accessory 4800 which is similar in configuration to accessory 4700 but in which the lower bar comprises double-cylinder configuration 4820. The double-cylinder configuration includes a pair of curved clamping surfaces 4823 spaced apart by a trough 4821 configured to receive a portion of the patient-facing side of the collapsible portion and/or configured to receive part of the upper bar 4818. In the collapsed configuration, the upper bar 4818 is moved toward the trough 4821 and may become seated within the trough 4821. This configuration may advantageously provide several pinch points at which the collapsible portion is pinched or clamped closed. These include a separate point between the upper bar 4818 and each of the two curved clamping surfaces 4823 and a pinch point between the upper bar 4818 and the trough 4821. In the collapsed configuration, the multiple pinch points provided by accessory 4800 may help to manipulate the lumen of the collapsible portion into a tortuous or kinked pathway which further increases flow resistance in order to lower the residual flow rate. The accessory 4800 could be inverted such that the double-cylinder configuration 4820 is the ‘upper’ bar and the bar 4818 is the ‘lower’ bar. In a variation of the accessory 4800, both the upper and lower bars comprise the double-cylinder configuration 4820.

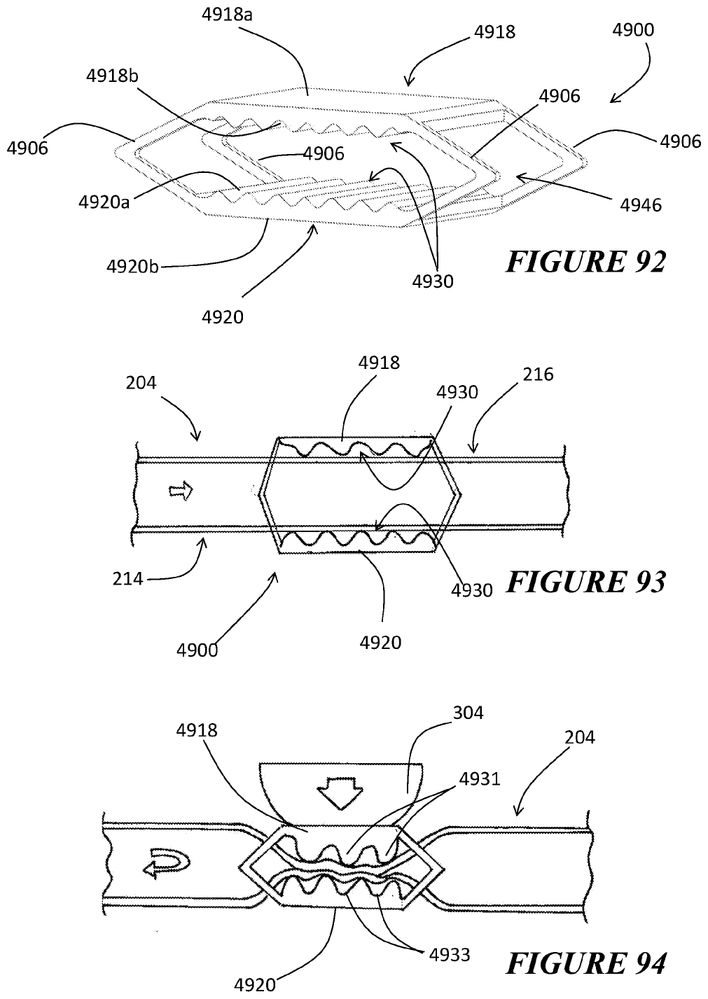

Turning to FIGS. 92 to 94, there is illustrated an accessory 4900 according to another embodiment of the present disclosure. Accessory 4900 includes a pair of clamping plates comprising an upper clamping plate 4918 and a lower clamping plate 4920. The upper and lower clamping plates 4918, 4920 are connected in spaced apart relation by four flexibly resilient v-shaped linkages 4906 which are similar in configuration to linkages 4606 discussed above with reference to FIGS. 87-89. The resilient linkages 4906 collectively provide a biasing configuration which retains the upper and lower clamping plates 4918, 4920 in the normally spaced apart configuration shown in FIGS. 92 and 93. The clamping plates 4918, 4920 are rectangular in profile but could have other shape profiles. The upper clamping plate 4918 includes a non-patient-facing surface 4918a and a patient-facing surface 4918b. The lower clamping plate 4920 includes a non-patient-facing surface 4918a and a patient-facing surface 4918b.

The upper clamping plate 4918 includes a concentration formation 4930 comprising a plurality of ribs on the patient-facing surface 4918b. The lower clamping plate 4920 includes an equivalent concentration formation 4930 on the non-patient-facing surface 4920a. In some embodiments, only one of the upper or lower clamping plates 4918, 4920 comprises a concentration formation 4930. The void between the opposing clamping plates 4918 and 4920 is bounded by the linkages 4906 and defines an opening 4946 configured for the collapsible portion to extend therethrough. In an alternative embodiment (not illustrated) the concentration formation 4930 comprises a series of discrete protrusions such as a series of teeth which creates multiple tortuous paths in the collapsed configuration.

Turning to FIG. 94, the accessory 4900 is fitted to the collapsible portion 204 which extends through the opening 4946 such that the accessory 4900 is attached to the collapsible portion 204. The concentration formation 4930 of the upper clamping plate 4918 faces and is in contact with the non-patient-facing surface 216 of the collapsible portion 204. The concentration formation 4930 of the lower clamping plate 4920 faces and is in contact with the patient-facing surface 214 of the collapsible portion 204. FIG. 94 illustrates an uncollapsed configuration and in which the accessory 4900 is in a normal i.e. at rest configuration.

FIG. 94 illustrates a collapsed configuration wherein an applied force of a bag mask cuff 304 onto the non-patient-facing surface 4918a of the upper clamping plate 4918 has resiliently deformed the accessory 4900 away from its normal configuration and into a clamped configuration wherein the upper clamping plate 4918 has moved toward the lower clamping plate 4920 thereby clamping the collapsible portion 204 between the opposing concentration formations 4930. The plurality of ribs in the concentration formations 4930 provide a series of pinch points at which the applied force is concentrated onto the collapsible portion 204. As shown in FIG. 34, the opposing concentration formations 4930 manipulate the lumen of the collapsible portion 204 into a tortuous path comprising a series of bends and/or kinks and/or pinch points.

Upon release of the applied force from the mask cuff 304, the resiliency of the linkages 4906, the resiliency of the collapsible portion 204 and/or the pressure of the respiratory airflow provided through the collapsible portion 204 will urge the accessory 4900 to return to the normal configuration shown in FIGS. 92 and 93.

According to a particular embodiment, the opposing concentration formations 4930 may be configured such that the peaks of the ribs on one of the concentration formations 4930 are aligned for receipt with the troughs of the opposing concentration formations. For example, with reference to FIG. 34 the peaks 4931 of the upper clamping plate 4918 are aligned with the troughs 4933 of the lower clamping plate 4920. In some embodiments, the peaks of the upper clamping plate 4918 are aligned with the peaks of the lower clamping plate 4920.

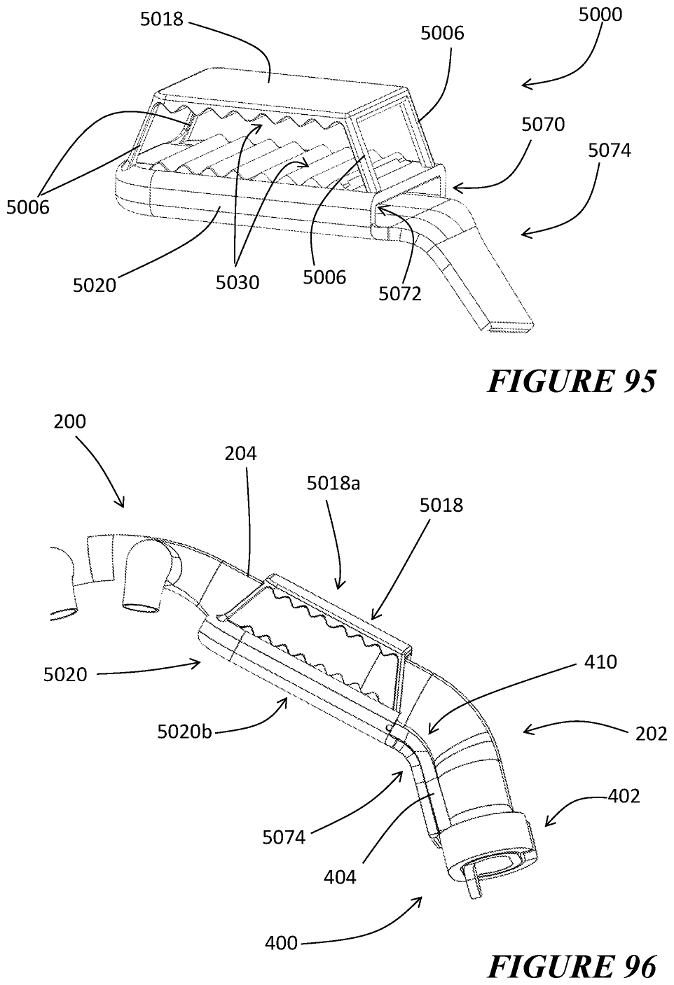

Turning to FIG. 95, there is illustrated an accessory 5000 which is similar in configuration to accessory 4900 in that accessory 5000 includes an upper clamping plate 5018 and a lower clamping plate 5020, each clamping plate including a concentration formation 5030 comprising a plurality ribs. In some embodiments, the upper and lower clamping plates are of different size and/or shape. Accessory 5000 differs from accessory 4900 in that accessory 5000 includes linear linkages 5006 instead of the V-shaped linkages 4906. Linear linkages 5006 collectively provide a biasing configuration which retains the upper and lower clamping plates 5018, 5020 in the normally spaced apart configuration shown in FIGS. 95 and 96. Accessory 5000 is also configured for cooperation with a backing plate such as that backing plate 3504 illustrated in FIG. 66. In particular, the lower clamping plate 5020 is configured with an opening 5070 leading to an internal void 5072. The internal void is configured in size and shape to receive a portion of the backing plate 3504 of accessory 3500. It will be appreciated that the void 5072 could also be configured to receive a backing plate other than the exact configuration illustrated with respect to accessory 3500. In some embodiments, the lower clamping plate 5020 is removably engageable with a backing plate via the internal void 5072.

The accessory 5000 further includes a curved portion 5074 extending from the lower clamping plate 5020. The curved portion is configured to conform and nest with the curved portion 3510 of the backing plate 3504 when backing plate 3504 is inserted into the opening 5070. In some embodiments, the curved portion 5074 is formed of a flexibly resilient material which is soft, and may act as a cushion between the rigid backing plate 3504 and the patient's face.

FIG. 96 illustrates accessory 5000 in cooperative operation with accessory 3500 and wherein each of accessory 5000 and accessory 3500 are also attached to the patient interface 200. Accessory 3500 is connected to the gas delivery conduit 202 in the manner discussed in the foregoing via the C-shaped clip 3502. Accessory 5000 is attached to the collapsible portion 204 in the manner discussed above with respect to accessory 4900 and wherein the collapsible portion 204 extends through an opening in accessory 5000 between the upper and lower clamping plates 5018, 5020 and between the linkages 5006.

The assembly and cooperation of accessory 3500 and accessory 5000, as illustrated in FIG. 96, provides a robust attachment to the patient interface 200 by virtue of there being two discrete attachments onto the gases conduit 202. Furthermore, the individual advantages provided by each accessory 3500 and 5000 may cooperate to improve overall performance. For example, the backing plate 3504 may operate to provide a rigid support surface which shields the patient's face from pressure of an applied force. The backing plate 3504 may also provide a rigid support surface against which the applied force is pressed to thereby facilitate collapse. Simultaneously, the accessory 5000 may facilitate an improved collapse of the collapsible portion 204 beyond what would be provided by accessory 3500 alone by virtue of the opposing clamping plates 5018, 5020 being compressed together to pinch the collapsible portion between the opposing concentration formations 5030.

The cooperative combination of accessory 3500 and accessory 5000 may also enable the convenient use of different materials having different material properties. In this manner, various material properties can be utilised in an advantageous way but also allowing for each accessory to be formed of a single material. For example, the accessory 3500 may be formed of a relatively rigid material providing a rigid support and responsive when the external force is applied. The accessory 3500 may therefore be formed of a single rigid material which improves manufacturing efficiency and reduces cost. Similarly, the accessory 5000 may be formed of a single material that is flexibly resilient. That is, the upper and lower clamping members 5018 and 5020 may be formed of the same material as the flexible linkages 5006. The use of a single material in each of the two accessories may reduce and simplify manufacturing costs as well as improve article quality insofar as the manufacturing process is simplified. In other embodiments, the accessory may be produced using over moulding to make part or all of the accessory from different materials.

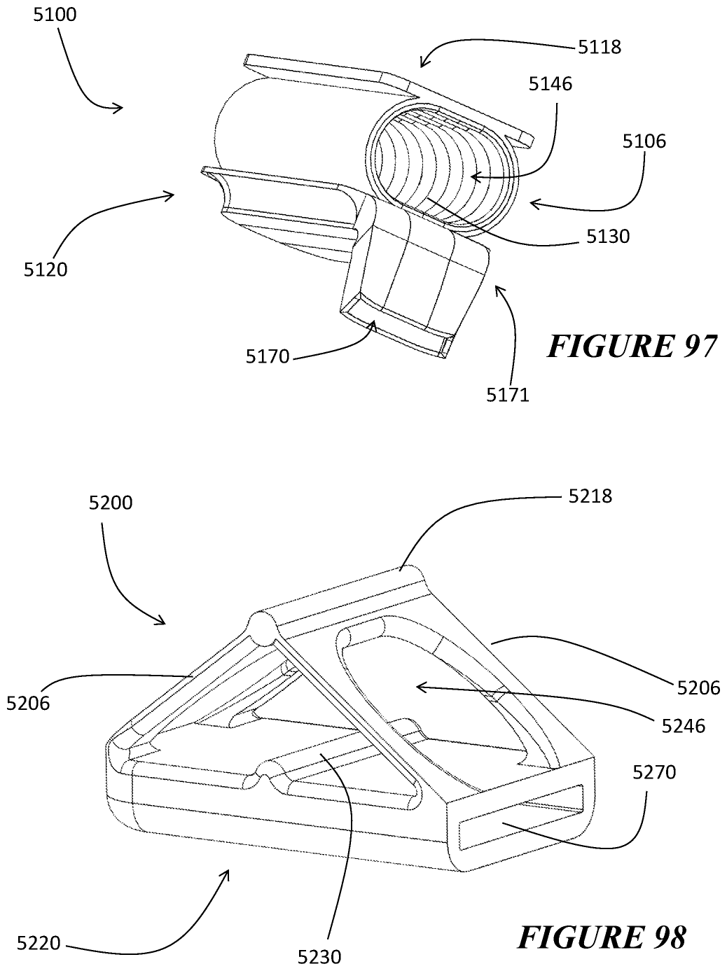

FIGS. 97 and 98 illustrated alternatives embodiments to the accessory 5000 shown in FIGS. 95 and 96.

FIG. 97 illustrates an accessory 5100 comprising a pair of collapsible clamping members comprising an upper clamping member 5118 and a lower clamping member 5120 which are connected and normally spaced apart by a resilient biasing configuration comprising a resilient tubular portion 5106. The tubular portion 5106 includes an opening 5146 for the collapsible portion to be received and extend therethrough, in use. An inner surface of the tubular portion a concentration formation 5130 comprising a plurality of ribs which circumscribe the opening 5146 and, in use, concentrate force onto the collapsible portion.

The lower clamping member 5120 includes a neck 5171 which includes an opening 5170 leading to an internal void (not shown) inside the lower clamping member 5120. The neck 5171 includes a pre-formed curve corresponding to a pre-formed curve of the backing plate 3504 of accessory 3500. The internal void and the opening 5170 are configured to receive a distal end of backing plate 3504 of accessory 3500. In use, the distal end of the backing plate 3504 (i.e. the end opposite to the clip 3502) is inserted through opening 5170 such that the accessory 5100 is fitted onto the backing plate 3504. The lower clamping member 5120 may be formed of a resilient material which allows resilient deformation of the curved neck 5171 to assist in locating the backing plate 3504 within the internal void.

In use, the accessory 5100 of FIG. 97 is cooperatively fitted together with the backing plate 3504, similar to the assembly of accessory 5000 and backing plate 3504 which is illustrated in FIG. 96. An externally applied force such as a force from an applied bag mask contacts the upper clamping member 5118 which is compressed toward the lower clamping member 5120. The ribs of the concentration formation 5130 concentrate the applied load onto the collapsible portion of the patient interface and facilitate collapse of the collapsible portion, similar to the collapsed configurations illustrated in FIG. 34. Upon release of the applied external force, the resilience of the tubular portion 5106, the resiliency of the collapsible portion 204 and/or the pressure of the respiratory airflow provided through the collapsible portion 204 urges the upper and lower clamping members 5118, 5120 to return to their normally spaced apart configuration shown in FIG. 97 which corresponds to an uncollapsed configuration of the collapsible portion.

Turning to FIG. 98, there is illustrated an accessory 5200 which provides another alternative to the accessories 5000 and 5100 that are configured for cooperative use with the accessory 3500. The accessory 5200 includes an upper clamping member 5218 comprising a cylindrical portion and a lower clamping member 5220 comprising a base plate having an opening 5270 leading to an internal void configured to receive the distal end of backing plate 3504 of accessory 3500. The lower clamping member 5220 includes a rib 5230. The upper and lower clamping members 5218, 5220 are connected and normally spaced apart by a resilient biasing configuration comprising a pair of collapsible walls 5206 which are angled with respect to the lower clamping member 5220 and form the generally triangular configuration shown in FIG. 98. Each of the collapsible walls 5206 includes an opening 5246 for, in use, receiving the collapsible portion of the patient interface.

In use, the accessory 5200 is fitted onto the backing plate 3504 of accessory 3500 in a similar configuration to that which is illustrated in FIG. 96 with respect to accessory 5000. Upon application of an external force onto the upper clamping member 5218, the collapsible walls 5206 are collapsed via resilient deformation to permit movement of the upper clamping member 5218 toward the rib 5230. The collapsible portion is thereby clamped between the upper clamping member 5218 and the rib 5230 of the lower clamping member 5220 which induces collapse of the collapsible portion and thereby reducing the flow of respiratory gases through the collapsible portion. Upon release of the externally applied force from the upper clamping member 5218, the resiliency of the collapsible walls 5206, the resiliency of the collapsible portion 204 and/or the pressure of the respiratory airflow provided through the collapsible portion 204 urges the upper and lower clamping members 5218, 2020 to return to the normally spaced apart configuration illustrated in FIG. 98.

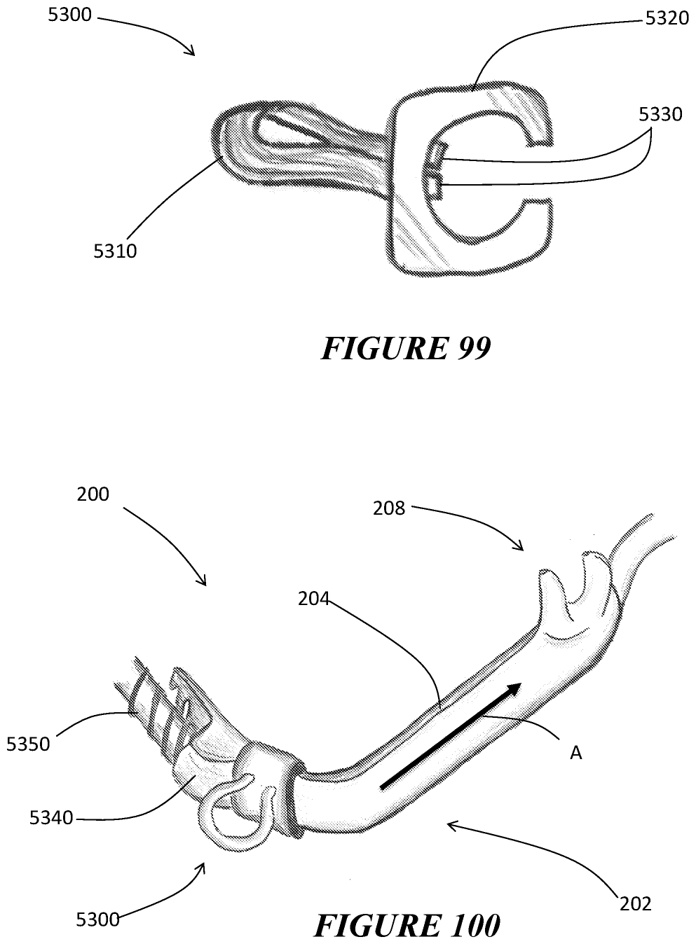

Turning to FIG. 99, there is illustrated an accessory 5300 according to another embodiment of the present disclosure. The accessory 5300 includes a gripping portion comprising a flexible loop 5310 and an attachment configuration comprising a C-shaped clip 5320 configured to attach the accessory 5300 to a portion of the gases delivery conduit of a patient interface. The flexible loop 5310 extends from the clip 5320 and is configured to allow, in use, allows digital application of a pulling force from a user's hand or fingers to the patient interface in a direction away from the patient's face in order to facilitate collapse or folding of the collapsible portion. The clip 5320 is formed of a resilient material to allow snap-fit engagement with the portion of the gases delivery conduit. As shown in FIG. 99, the ends 5330 of the loop 5310 are attached through the clip 5320.

FIG. 100 illustrates the accessory 5300 fitted to patient interface 200 comprising a nasal cannula comprising a pair of nasal prongs 208 configured for delivering respiratory gases to a patient's nares. The accessory 5300 is fitted to a part of the gases delivery conduit 202 comprising a gas path connector 5340. In some embodiments, the accessory 5300 is fitted to a rigid part of the gases delivery conduit 202. The gas path connector 5340 connects the collapsible portion to a gas supply conduit 5350. The accessory 5300 is attached to the patient interface at a position upstream of the collapsible portion 204 with respect to the direction of respiratory gases which flow from the supply conduit 5350 toward the nasal prongs 208, as indicated by flow arrow ‘A’.

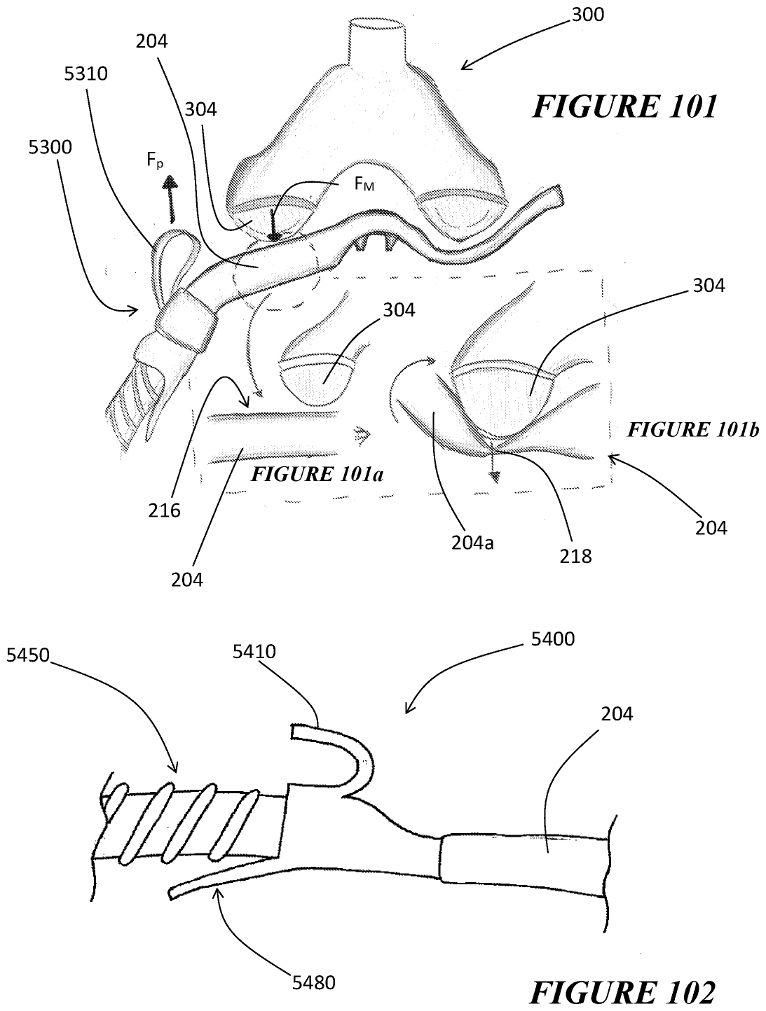

Turning to FIGS. 101 and 101a, the operation of the accessory 5300 is illustrated in more detail. FIG. 101 illustrates another perspective of the arrangement illustrated in FIG. 100 with accessory 5300 fitted to patient interface 200. A patient mask 300 is pressed down onto the patient's face (not shown) such that the inflatable cuff 304 of the bag mask 300 is pressed onto the collapsible portion 204 applying a mask force F_{M} onto the collapsible portion 204 in the direction of the patient's face. Simultaneously, a pulling force F_{P} from the user's finger is applied to the finger loop 5310 in a direction away from the patient's face.

FIGS. 101a and 101b provides a closer perspective of the engagement between the mask cuff 304 and the collapsible portion 204. FIG. 101a illustrates the mask cuff 304 and collapsible portion 204 immediately prior to contact between the mask cuff 304 and a non-patient-facing surface 216 of the collapsible portion 204. FIG. 101b illustrates the mask cuff 304 having been pressed down onto the collapsible portion and whereupon the pulling for F_{P} has been applied to the accessory 5300 causing an upstream portion 204a of the collapsible portion 204 which is located upstream of the mask cuff 304 to fold outward away from the patient's face and in the direction of the pulling force F_{P}. As shown in FIG. 101b, an obstruction comprising a kink 218 is formed in the collapsible portion 204. The kink 218 is formed as a result of the simultaneously application of forces F_{M} and F_{P} which cooperate to collapse the collapsible portion 204 and partially fold the upstream portion 204a around the mask cuff 304 in the clockwise direction indicated in FIG. 101b.

FIG. 102 illustrates an accessory 5400 which comprises a variation of the finger-loop accessory 5300 discussed above and illustrated in FIGS. 99-101b. The accessory 5400 comprises a gas path connector 5400 configured to connect a gas supply conduit 5450 to the collapsible portion 204. The gas path connector 5400 includes a finger hook 5410 facilitating digital application of a pulling force away from the patient's face. The finger hook 5410 may be continuous (similar to flexible loop 5310) or discontinuous (as shown in FIG. 102). The accessory 5400 provides an embodiment of the present disclosure in which the finger-loop accessory 5300 is integrally formed into a component of the patient interface. The accessory 5400 therefore serves more than one function for example, firstly, a connector in the gases delivery conduit, and secondly as a component allowing application of a pulling force to facilitate collapse of the collapsible portion. Additionally, the accessory 5400 may provide a portion 5480 for a headstrap attachment

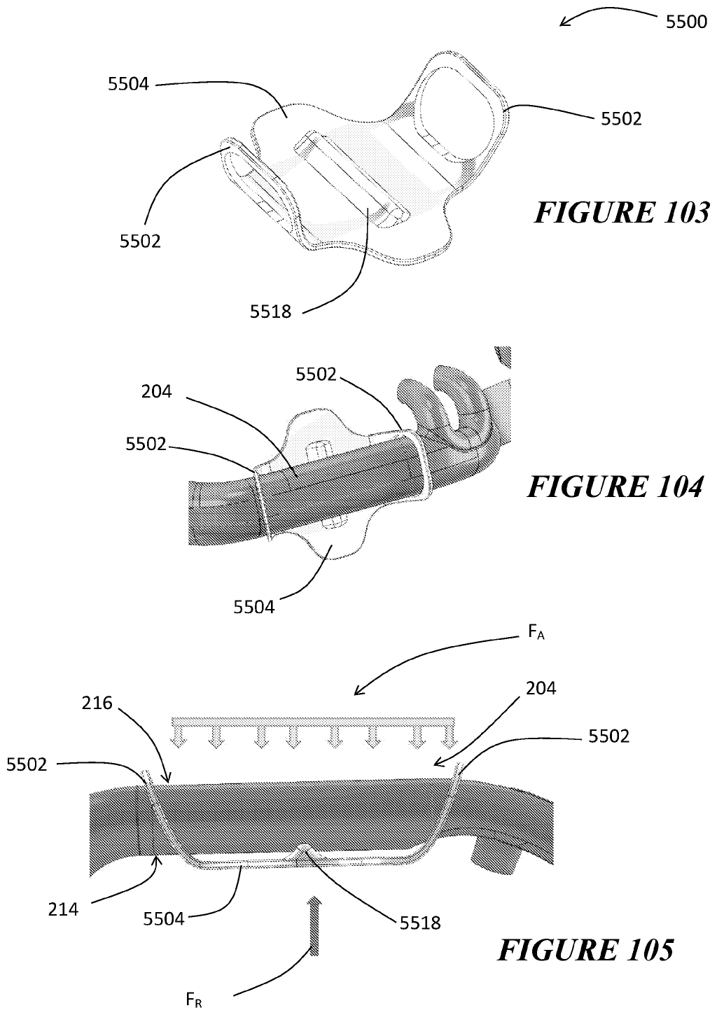

FIGS. 103 to 105 illustrated an accessory 5500 according to another embodiment of the present disclosure. The accessory 5500 includes a back plate 5504 with a single rib 5518. An attachment configuration comprising a pair of loops 5502 extending from the back plate 5504. The loops 5502 are configured for the collapsible portion 204 to extend through the pair of loops 5502, as shown in FIG. 104.

Turning to FIG. 105, the back plate 5504 is located below a patient-facing-surface 214 of the collapsible portion 204 and is, in use, located between the collapsible portion 204 and the patient's face. The patient-facing-surface 214 overlies the rib 5518. An applied force F_{A} from a cuff of a patient mask is applied between the loops 5502 onto a non-patient-facing surface 216. A reactionary force F_{R} is applied to the patient-facing surface 214 by the rib 5518 which concentrates the applied force F_{A} onto a smaller contact area at the tip of rib 5518. The reactionary force F_{R} thereby applies a pressure onto the patient-facing-surface 214 which is greater than the pressure applied onto the non-patient-facing surface 216 by the applied force F_{A}. In a particular embodiment, the back plate 5502 is configured in size or shape so as to overlie and be supported by hard sections of the patient's face (for example, areas of bone structure) in order to support the back plate 5504 and provide adequate resistance to the applied force F_{A}.

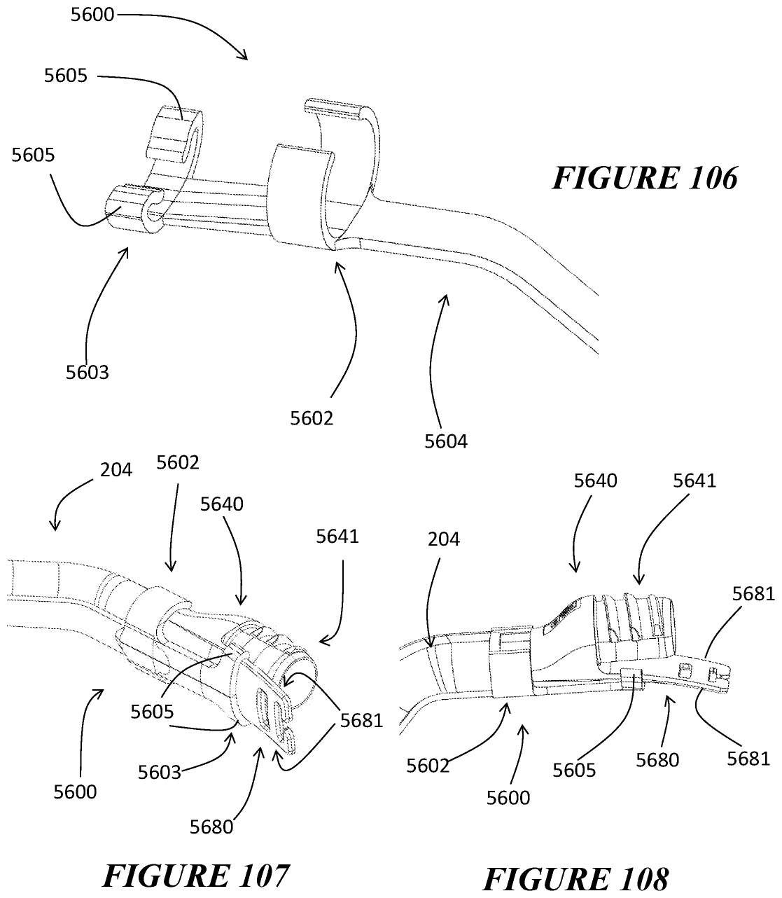

Turning to FIG. 106, there is illustrated an accessory 5600 according to another embodiment of the present disclosure. The accessory 5600 includes a backing plate 5504 extending from an attachment configuration comprising a C-shaped clip 5602 in a similar configuration to accessory 3500 of FIG. 66. However, accessory 5600 includes an additional attachment configuration comprising a second clip 5603 configuration configured to connect with a part of a patient interface. The second clip 5603 is, in particular, configured to connect with a gas path connector 5640 of the patient interface as is illustrated in FIGS. 107 and 108. The gas path connector 5640 includes a downstream connector which connected to the collapsible portion 204 and an upstream connector comprising a male-type connector 5641 which is shown in FIGS. 107 and 48 and which is configured for connection to a gases supply conduit (not shown). The gas path connector 5550 includes a flange comprising an elongate projection 5680 which extends in a similar direction to the male-type connector 5641 and generally in the upstream direction. The projection 5680 provides a connection point for a headstrap (not shown) used to secure the patient interface to the patient's face.

The second clip 5603 includes a pair of resilient U-shaped arms 5605 configured to clip onto longitudinal edges 5681 of the projection 5680 on the gas path connector 5640. The second clip 5603 is configured to help retain accessory 5600 in position with respect to the gases delivery conduit. In particular, the second clip 5603 is configured to resist rotational movement of the accessory 5600 around the circumference of the gases delivery conduit.

In other embodiments, the second clip 5603 may connect with another region of the same portion to which the C-shaped clip 5602 connects, where the second clip 5603 may be spaced away from the C-shaped clip 5602. In such embodiments, the second clip 5603 may comprise a shape that is substantially similar to that of the C-shaped clip 5602, i.e. the accessory 5600 comprises two C-shaped clips. In other embodiments, the second clip 5603 comprises a shape that corresponds to the portion of the patient interface to which it connects. In other embodiments, the C-shaped clip 5602 and/or second clip 5603 may be an attachment means which include but are not limited to adhesives, hook-and-loop fasteners, plug and socket attachments, etc.

It will be appreciated that certain embodiments of an accessory according to the present disclosure may have a one-part or unitary configuration whereas other embodiments comprise a multi-part or assembly configuration. For example, the embodiments of accessory 3500, accessory 3600, accessory 3600A, accessory 3700, accessory 3800, accessory 3900, accessory 4000, accessory 4500, accessory 4600, accessory 4700, accessory 4800, accessory 4900, accessory 5000 and accessory 5500 may comprise a single component and might be produced via a single manufacturing technique such as injection moulding. Other embodiments such as accessory 4100, accessory 4200, accessory 4300, accessory 4400, accessories 4400A-D and accessory 5300 may include multiple and/or moving parts or assemblies of parts and may therefore require production in different or separate manufacturing techniques.

The accessory of the present disclosure may be produced using one or more of manufacturing techniques including injection molding, overmolding, and additive manufacturing such as 3D printing. In some embodiments, injection moulding could be used to produce an accessory made from more than one material. In embodiments where a part is formed of metallic material then this part may be producing using machining.

The inventions may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Moreover, not all of the features, aspects and advantages are necessarily required to practice the present disclosure. Accordingly, the scope of the present disclosure is intended to be defined only by the claims that follow.

1. A patient interface comprising:
a gases delivery interface configured to deliver an apparatus gas flow to a patient, the gases delivery interface comprising:
a delivery outlet to deliver the apparatus gas flow to the patient; and
a gases delivery side member extending from a first side of the delivery outlet and comprising an apparatus gases flow path in fluid communication with the delivery outlet, the gases delivery side member comprising a collapsible portion movable upon application of a collapsing force from a normally open configuration to a collapsed configuration in which the apparatus gases flow path is reduced or closed in order to reduce or stop the apparatus gas flow through the apparatus gases flow path; and
a gas sampling interface comprising:
a sampling inlet configured to receive a patient gas flow at the patient;
a sampling outlet configured for delivery of the patient gas flow away from the patient; and
a sampling conduit in fluid communication with the sampling inlet and the sampling outlet, the sampling conduit configured to remain open to maintain fluid communication between the sampling inlet and sampling outlet when the collapsible portion is moved to the collapsed configuration.

2. (canceled)

3. (canceled)

4. The patient interface according to claim 1, wherein the gases delivery interface further comprises a non-delivery side member extending from a second side of the delivery outlet that is opposite to the first side, the non-delivery side member comprising a headstrap end that is configured for coupling to a headstrap, the non-delivery side member also comprising a patient-facing wall and a non-patient-facing wall.

5. The patient interface according to claim 1, wherein the gas sampling interface provided is to the gases delivery side member, the gases delivery side member comprises a delivery inlet at one end to receive the apparatus gas flow, and the gases delivery side member comprises a patient-facing wall and a non-patient-facing wall.

6. The patient interface according to claim 5, wherein the sampling inlet is located proximate to the delivery outlet and the sampling outlet is located proximate to the delivery inlet.

7. The patient interface according to claim 5, wherein the sampling conduit comprises a sampling lumen for the patient gas flow and the gases delivery side member comprises a gases delivery lumen for the apparatus gas flow.

8. The patient interface according to claim 5, wherein the sampling conduit extends alongside an external surface of the gases delivery side member.

9. The patient interface according to claim 7, wherein the sampling conduit is configured to be stiffer than the collapsible portion to maintain the shape of the sampling lumen upon application of the collapsing force, and the sampling conduit is one of:
formed of a material having sufficient material stiffness to retain the shape of the sampling lumen,
formed of a material that has greater material stiffness than a material of the collapsible portion, or
configured via geometric features to be stiffer than the collapsible portion.

10. (canceled)

11. (canceled)

12. (canceled)

13. The patient interface according to claim 1, wherein in the normally open configuration, the gases delivery interface is configured to allow an apparatus gas flow rate of about 20 Umin to about 90 Umin through the apparatus gases flow path.

14. The patient interface according to claim 1, wherein in the collapsed configuration, the patient interface is configured to allow an apparatus gas flow rate through the apparatus gases flow path that is at least 20 times greater than a patient gas flow rate through the gas sampling interface.

15. The patient interface according to claim 1, wherein in the collapsed configuration, the gases delivery interface is configured to allow an apparatus gas flow rate of less than about 10 L/min through the apparatus gases flow path, and the gas sampling interface is configured to allow a patient gas flow rate of less than about 500 mL/min, optionally about 40 mL/min to about 500 mL/min through the gas sampling interface.

16. The patient interface according to claim 1, wherein the patient interface comprises a single sampling conduit.

17. The patient interface according to claim 1, wherein the sampling conduit comprises a single lumen and the single lumen being a sampling lumen for the patient gas flow.

18. The patient interface according to claim 1, wherein the sampling inlet comprises a nasal inlet and a mouth inlet.

19. (canceled)

20. (canceled)

21. The patient interface according to claim 1, wherein the sampling conduit comprises a thicker wall than a wall of the collapsible portion.

22. The patient interface according to claim 1, wherein the sampling conduit comprises a wall of uniform thickness and the collapsible portion comprising a wall of non-uniform thickness.

23. The patient interface according to claim 1, wherein the collapsible portion comprises one or more thin-walled portions configured to facilitate bending or folding and thereby facilitate movement of the collapsible portion to the collapsed configuration.

24. The patient interface according to claim 1, wherein the gas sampling interface is formed of a different material than the collapsible portion.

25. The patient interface according to claim 24, wherein the gas sampling interface comprises at least one of silicone or a thermoplastic elastomer.

26. The patient interface according to claim 1, wherein the sampling conduit has a width that is less than a width of the collapsible portion.
